SEQUENCE LISTING (70 sequences)

SEQ ID NO: 1 - protein, 602 aa, unidentified
  source 1..602: note = Nipah virus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 2 - protein, 4 aa, Synthetic construct
PSAP

SEQ ID NO: 3 - protein, 433 aa, Homo sapiens
MGRVQLFEISLSHGRVVYSPGEPLAGTVRVRLGAPLPFRAIRVTCIGSCGVSNKANDTAWVVEEGYFNSSLSLADKGSLPAGEHSFPFQFLLPATAPTSFEGPFGKIVHQVRAAIHTPRFSKDHKCSLVFYILSPLNLNSIPDIEQPNVASATKKFSYKLVKTGSVVLTASTDLRGYVVGQALQLHADVENQSGKDTSPVVASLLQKVSYKAKRWIHDVRTIAEVEGAGVKAWRRAQWHEQILVPALPQSALPGCSLIHIDYYLQVSLKAPEATVTLPVFIGNIAVNHAPVSPRPGLGLPPGAPPLVVPSAPPQEEAEAEAAAGGPHFLDPVFLSTKSHSQRQPLLATLSSVPGAPEPCPQDGSPASHPLHPPLCISTGATVPYFAEGSGGPVPTTSTLILPPEYSSWGYPYEAPPSYEQSCGGVEPSLTPES

SEQ ID NO: 4 - protein, 434 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQKVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 5 - protein, 433 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 6 - protein, 546 aa, unidentified
  source 1..546: note = Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 7 - protein, 922 aa, Homo sapiens
MATASPRSDTSNNHSGRLQLQVTVSSAKLKRKKNWFGTAIYTEVVVDGEITKTAKSSSSSNPKWDEQLTVNVTPQTTLEFQVWSHRTLKADALLGKATIDLKQALLIHNRKLERVKEQLKLSLENKNGIAQTGELTVVLDGLVIEQENITNCSSSPTIEIQENGDALHENGEPSARTTARLAVEGTNGIDNHVPTSTLVQNSCCSYVVNGDNTPSSPSQVAARPKNTPAPKPLASEPADDTVNGESSSFAPTDNASVTGTPVVSEENALSPNCTSTTVEDPPVQEILTSSENNECIPSTSAELESEARSILEPDTSNSRSSSAFEAAKSRQPDGCMDPVRQQSGNANTETLPSGWEQRKDPHGRTYYVDHNTRTTTWERPQPLPPGWERRVDDRRRVYYVDHNTRTTTWQRPTMESVRNFEQWQSQRNQLQGAMQQFNQRYLYSASMLAAENDPYGPLPPGWEKRVDSTDRVYFVNHNTKTTQWEDPRTQGLQNEEPLPEGWEIRYTREGVRYFVDHNTRTTTFKDPRNGKSSVTKGGPQIAYERGFRWKLAHFRYLCQSNALPSHVKINVSRQTLFEDSFQQIMALKPYDLRRRLYVIFRGEEGLDYGGLAREWFFLLSHEVLNPMYCLFEYAGKNNYCLQINPASTINPDHLSYFCFIGRFIAMALFHGKFIDTGFSLPFYKRMLSKKLTIKDLESIDTEFYNSLIWIRDNNIEECGLEMYFSVDMEILGKVTSHDLKLGGSNILVTEENKDEYIGLMTEWRFSRGVQEQTKAFLDGFNEVVPLQWLQYFDEKELEVMLCGMQEVDLADWQRNTVYRHYTRNSKQIIWFWQFVKETDNEVRMRLLQFVTGTCRLPLGGFAELMGSNGPQKFCIEKVGKDTWLPRSHTCFNRLDLPPYKSYEQLKEKLLFAIEETEGFGQE

SEQ ID NO: 8 - protein, 34 aa, Homo sapiens
ETLPSGWEQRKDPHGRTYYVDHNTRTTTWERPQP

SEQ ID NO: 9 - protein, 34 aa, Homo sapiens
QPLPPGWERRVDDRRRVYYVDHNTRTTTWQRPTM

SEQ ID NO: 10 - protein, 39 aa, Homo sapiens
ENDPYGPLPPGWEKRVDSTDRVYFVNHNTKTTQWEDPRT

SEQ ID NO: 11 - protein, 34 aa, Homo sapiens
EPLPEGWEIRYTREGVRYFVDHNTRTTTFKDPRN

SEQ ID NO: 12 - protein, 870 aa, Homo sapiens
MASASSSRAGVALPFEKSQLTLKVVSAKPKVHNRQPRINSYVEVAVDGLPSETKKTGKRIGSSELLWNEIIILNVTAQSHLDLKVWSCHTLRNELLGTASVNLSNVLKNNGGKMENMQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 13 - protein, 34 aa, Homo sapiens
DALPAGWEQRELPNGRVYYVDHNTKTTTWERPLP

SEQ ID NO: 14 - protein, 33 aa, Homo sapiens
PLPPGWEKRTDPRGRFYYVDHNTRTTTWQRPTA

SEQ ID NO: 15 - protein, 37 aa, Homo sapiens
HDPLGPLPPGWEKRQDNGRVYYVNHNTRTTQWEDPRT

SEQ ID NO: 16 - protein, 34 aa, Homo sapiens
PALPPGWEMKYTSEGVRYFVDHNTRTTTFKDPRP

SEQ ID NO: 17 - protein, 1319 aa, Homo sapiens
MAQSLRLHFAARRSNTYPLSETSGDDLDSHVHMCFKRPTRISTSNVVQMKLTPRQTALAPLIKENVQSQERSSVPSSENVNKKSSCLQISLQPTRYSGYLQSSNVLADSDDASFTCILKDGIYSSAVVDNELNAVNDGHLVSSPAICSGSLSNFSTSDNGSYSSNGSDFGSCASITSGGSYTNSVISDSSSYTFPPSDDTFLGGNLPSDSTSNRSVPNRNTTPCEIFSRSTSTDPFVQDDLEHGLEIMKLPVSRNTKIPLKRYSSLVIFPRSPSTTRPTSPTSLCTLLSKGSYQTSHQFIISPSEIAHNEDGTSAKGFLSTAVNGLRLSKTICTPGEVRDIRPLHRKGSLQKKIVLSNNTPRQTVCEKSSEGYSCVSVHFTQRKAATLDCETTNGDCKPEMSEIKLNSDSEYIKLMHRTSACLPSSQNVDCQININGELERPHSQMNKNHGILRRSISLGGAYPNISCLSSLKHNCSKGGPSQLLIKFASGNEGKVDNLSRDSNRDCTNELSNSCKTRDDFLGQVDVPLYPLPTENPRLERPYTFKDFVLHPRSHKSRVKGYLRLKMTYLPKTSGSEDDNAEQAEELEPGWVVLDQPDAACHLQQQQEPSPLPPGWEERQDILGRTYYVNHESRRTQWKRPTPQDNLTDAENGNIQLQAQRAFTTRRQISEETESVDNRESSENWEIIREDEATMYSNQAFPSPPPSSNLDVPTHLAEELNARLTIFGNSAVSQPASSSNHSSRRGSLQAYTFEEQPTLPVLLPTSSGLPPGWEEKQDERGRSYYVDHNSRTTTWTKPTVQATVETSQLTSSQSSAGPQSQASTSDSGQQVTQPSEIEQGFLPKGWEVRHAPNGRPFFIDHNTKTTTWEDPRLKIPAHLRGKTSLDTSNDLGPLPPGWEERTHTDGRIFYINHNIKRTQWEDPRLENVAITGPAVPYSRDYKRKYEFFRRKLKKQNDIPNKFEMKLRRATVLEDSYRRIMGVKRADFLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLHKPITLHDMESVDSEYYNSLRWILENDPTELDLRFIIDEELFGQTHQHELKNGGSEIVVTNKNKKEYIYLVIQWRFVNRIQKQMAAFKEGFFELIPQDLIKIFDENELELLMCGLGDVDVNDWREHTKYKNGYSANHQVIQWFWKAVLMMDSEKRIRLLQFVTGTSRVPMNGFAELYGSNGPQSFTVEQWGTPEKLPRAHTCFNRLDLPPYESFEELWDKLQMAIENTQGFDGVD

SEQ ID NO: 18 - protein, 34 aa, Homo sapiens
SPLPPGWEERQDILGRTYYVNHESRRTQWKRPTP

SEQ ID NO: 19 - protein, 34 aa, Homo sapiens
SGLPPGWEEKQDERGRSYYVDHNSRTTTWTKPTV

SEQ ID NO: 20 - protein, 34 aa, Homo sapiens
GFLPKGWEVRHAPNGRPFFIDHNTKTTTWEDPRL

SEQ ID NO: 21 - protein, 34 aa, Homo sapiens
GPLPPGWEERTHTDGRIFYINHNIKRTQWEDPRL

SEQ ID NO: 22 - protein, 955 aa, Homo sapiens
MATGLGEPVYGLSEDEGESRILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGLALPPPPASPGSRTSPQELSEELSRRLQITPDSNGEQFSSLIQREPSSRLRSCSVTDAVAEQGHLPPPSVAYVHTTPGLPSGWEERKDAKGRTYYVNHNNRTTTWTRPIMQLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 23 - protein, 27 aa, Homo sapiens
GWEEKVDNLGRTYYVNHNNRTTQWHRP

SEQ ID NO: 24 - protein, 29 aa, Homo sapiens
PSGWEERKDAKGRTYYVNHNNRTTTWTRP

SEQ ID NO: 25 - protein, 31 aa, Homo sapiens
PPGWEMRIAPNGRPFFIDHNTKTTTWEDPRL

SEQ ID NO: 26 - protein, 31 aa, Homo sapiens
PPGWEERIHLDGRTFYIDHNSKITQWEDPRL

SEQ ID NO: 27 - protein, 757 aa, Homo sapiens
MSNPGTRRNGSSIKIRLTVLCAKNLAKKDFFRLPDPFAKIVVDGSGQCHSTDTVKNTLDPKWNQHYDLYVGKTDSITISVWNHKKIHKKQGAGFLGCVRLLSNAISRLKDTGYQRLDLCKLNPSDTDAVRGQIVVSLQTRDRIGTGGSVVDCRGLLENEGTVYEDSGPGRPLSCFMEEPAPYTDSTGAAAGGGNCRFVESPSQDQRLQAQRLRNPDVRGSLQTPQNRPHGHQSPELPEGYEQRTTVQGQVYFLHTQTGVSTWHDPRIPSPSGTIPGGDAAFLYEFLLQGHTSEPRDLNSVNCDELGPLPPGWEVRSTVSGRIYFVDHNNRTTQFTDPRLHHIMNHQCQLKEPSQPLPLPSEGSLEDEELPAQRYERDLVQKLKVLRHELSLQQPQAGHCRIEVSREEIFEESYRQIMKMRPKDLKKRLMVKFRGEEGLDYGGVAREWLYLLCHEMLNPYYGLFQYSTDNIYMLQINPDSSINPDHLSYFHFVGRIMGLAVFHGHYINGGFTVPFYKQLLGKPIQLSDLESVDPELHKSLVWILENDITPVLDHTFCVEHNAFGRILQHELKPNGRNVPVTEENKKEYVRLYVNWRFMRGIEAQFLALQKGFNELIPQHLLKPFDQKELELIIGGLDKIDLNDWKSNTRLKHCVADSNIVRWFWQAVETFDEERRARLLQFVTGSTRVPLQGFKALQGSTGAAGPRLFTIHLIDANTDNLPKAHTCFNRIDIPPYESYEKLYEKLLTAVEETCGFAVE

SEQ ID NO: 28 - protein, 34 aa, Homo sapiens
PELPEGYEQRTTVQGQVYFLHTQTGVSTWHDPRI

SEQ ID NO: 29 - protein, 34 aa, Homo sapiens
GPLPPGWEVRSTVSGRIYFVDHNNRTTQFTDPRL

SEQ ID NO: 30 - protein, 748 aa, Homo sapiens
MSNPGGRRNGPVKLRLTVLCAKNLVKKDFFRLPDPFAKVVVDGSGQCHSTDTVKNTLDPKWNQHYDLYIGKSDSVTISVWNHKKIHKKQGAGFLGCVRLLSNAINRLKDTGYQRLDLCKLGPNDNDTVRGQIVVSLQSRDRIGTGGQVVDCSRLFDNDLPDGWEERRTASGRIQYLNHITRTTQWERPTRPASEYSSPGRPLSCFVDENTPISGTNGATCGQSSDPRLAERRVRSQRHRNYMSRTHLHTPPDLPEGYEQRTTQQGQVYFLHTQTGVSTWHDPRVPRDLSNINCEELGPLPPGWEIRNTATGRVYFVDHNNRTTQFTDPRLSANLHLVLNRQNQLKDQQQQQVVSLCPDDTECLTVPRYKRDLVQKLKILRQELSQQQPQAGHCRIEVSREEIFEESYRQVMKMRPKDLWKRLMIKFRGEEGLDYGGVAREWLYLLSHEMLNPYYGLFQYSRDDIYTLQINPDSAVNPEHLSYFHFVGRIMGMAVFHGHYIDGGFTLPFYKQLLGKSITLDDMELVDPDLHNSLVWILENDITGVLDHTFCVEHNAYGEIIQHELKPNGKSIPVNEENKKEYVRLYVNWRFLRGIEAQFLALQKGFNEVIPQHLLKTFDEKELELIICGLGKIDVNDWKVNTRLKHCTPDSNIVKWFWKAVEFFDEERRARLLQFVTGSSRVPLQGFKALQGAAGPRLFTIHQIDACTNNLPKAHTCFNRIDIPPYESYEKLYEKLLTAIEETCGFAVE

SEQ ID NO: 31 - protein, 34 aa, Homo sapiens
NDLPDGWEERRTASGRIQYLNHITRTTQWERPTR

SEQ ID NO: 32 - protein, 34 aa, Homo sapiens
PDLPEGYEQRTTQQGQVYFLHTQTGVSTWHDPRV

SEQ ID NO: 33 - protein, 34 aa, Homo sapiens
GPLPPGWEIRNTATGRVYFVDHNNRTTQFTDPRL

SEQ ID NO: 34 - protein, 903 aa, Homo sapiens
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSENGVSLCLPRLECNSAISAHCNLCLPGLSDSPISASRVAGFTGASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 35 - protein, 34 aa, Homo sapiens
APLPPGWEQRVDQHGRVYYVDHVEKRTTWDRPEP

SEQ ID NO: 36 - protein, 34 aa, Homo sapiens
EPLPPGWERRVDNMGRIYYVDHFTRTTTWQRPTL

SEQ ID NO: 37 - protein, 34 aa, Homo sapiens
GPLPPGWEKRTDSNGRVYFVNHNTRITQWEDPRS

SEQ ID NO: 38 - protein, 34 aa, Homo sapiens
KPLPEGWEMRFTVDGIPYFVDHNRRTTTYIDPRT

SEQ ID NO: 39 - protein, 1606 aa, Homo sapiens
MLLHLCSVKNLYQNRFLGLAAMASPSRNSQSRRRCKEPLRYSYNPDQFHNMDLRGGPHDGVTIPRSTSDTDLVTSDSRSTLMVSSSYYSIGHSQDLVIHWDIKEEVDAGDWIGMYLIDEVLSENFLDYKNRGVNGSHRGQIIWKIDASSYFVEPETKICFKYYHGVSGALRATTPSVTVKNSAAPIFKSIGADETVQGQGSRRLISFSLSDFQAMGLKKGMFFNPDPYLKISIQPGKHSIFPALPHHGQERRSKIIGNTVNPIWQAEQFSFVSLPTDVLEIEVKDKFAKSRPIIKRFLGKLSMPVQRLLERHAIGDRVVSYTLGRRLPTDHVSGQLQFRFEITSSIHPDDEEISLSTEPESAQIQDSPMNNLMESGSGEPRSEAPESSESWKPEQLGEGSVPDGPGNQSIELSRPAEEAAVITEAGDQGMVSVGPEGAGELLAQVQKDIQPAPSAEELAEQLDLGEEASALLLEDGEAPASTKEEPLEEEATTQSRAGREEEEKEQEEEGDVSTLEQGEGRLQLRASVKRKSRPCSLPVSELETVIASACGDPETPRTHYIRIHTLLHSMPSAQGGSAAEEEDGAEEESTLKDSSEKDGLSEVDTVAADPSALEEDREEPEGATPGTAHPGHSGGHFPSLANGAAQDGDTHPSTGSESDSSPRQGGDHSCEGCDASCCSPSCYSSSCYSTSCYSSSCYSASCYSPSCYNGNRFASHTRFSSVDSAKISESTVFSSQDDEEEENSAFESVPDSMQSPELDPESTNGAGPWQDELAAPSGHVERSPEGLESPVAGPSNRREGECPILHNSQPVSQLPSLRPEHHHYPTIDEPLPPNWEARIDSHGRVFYVDHVNRTTTWQRPTAAATPDGMRRSGSIQQMEQLNRRYQNIQRTIATERSEEDSGSQSCEQAPAGGGGGGGSDSEAESSQSSLDLRREGSLSPVNSQKITLLLQSPAVKFITNPEFFTVLHANYSAYRVFTSSTCLKHMILKVRRDARNFERYQHNRDLVNFINMFADTRLELPRGWEIKTDQQGKSFFVDHNSRATTFIDPRIPLQNGRLPNHLTHRQHLQRLRSYSAGEASEVSRNRGASLLARPGHSLVAAIRSQHQHESLPLAYNDKIVAFLRQPNIFEMLQERQPSLARNHTLREKIHYIRTEGNHGLEKLSCDADLVILLSLFEEEIMSYVPLQAAFHPGYSFSPRCSPCSSPQNSPGLQRASARAPSPYRRDFEAKLRNFYRKLEAKGFGQGPGKIKLIIRRDHLLEGTFNQVMAYSRKELQRNKLYVTFVGEEGLDYSGPSREFFFLLSQELFNPYYGLFEYSANDTYTVQISPMSAFVENHLEWFRFSGRILGLALIHQYLLDAFFTRPFYKALLRLPCDLSDLEYLDEEFHQSLQWMKDNNITDILDLTFTVNEEVFGQVTERELKSGGANTQVTEKNKKEYIERMVKWRVERGVVQQTEALVRGFYEVVDSRLVSVFDARELELVIAGTAEIDLNDWRNNTEYRGGYHDGHLVIRWFWAAVERFNNEQRLRLLQFVTGTSSVPYEGFAALRGSNGLRRFCIEKWGKITSLPRAHTCFNRLDLPPYPSYSMLYEKLLTAVEETSTFGLE

SEQ ID NO: 40 - protein, 33 aa, Homo sapiens
PLPPNWEARIDSHGRVFYVDHVNRTTTWQRPTA

SEQ ID NO: 41 - protein, 34 aa, Homo sapiens
LELPRGWEIKTDQQGKSFFVDHNSRATTFIDPRI

SEQ ID NO: 42 - protein, 1572 aa, Homo sapiens
MASSAREHLLFVRRRNPQMRYTLSPENLQSLAAQSSMPENMTLQRANSDTDLVTSESRSSLTASMYEYTLGQAQNLIIFWDIKEEVDPSDWIGLYHIDENSPANFWDSKNRGVTGTQKGQIVWRIEPGPYFMEPEIKICFKYYHGISGALRATTPCITVKNPAVMMGAEGMEGGASGNLHSRKLVSFTLSDLRAVGLKKGMFFNPDPYLKMSIQPGKKSSFPTCAHHGQERRSTIISNTTNPIWHREKYSFFALLTDVLEIEIKDKFAKSRPIIKRFLGKLTIPVQRLLERQAIGDQMLSYNLGRRLPADHVSGYLQFKVEVTSSVHEDASPEAVGTILGVNSVNGDLGSPSDDEDMPGSHHDSQVCSNGPVSEDSAADGTPKHSFRTSSTLEIDTEELTSTSSRTSPPRGRQDSLNDYLDAIEHNGHSRPGTATCSERSMGASPKLRSSFPTDTRLNAMLHIDSDEEDHEFQQDLGYPSSLEEEGGLIMFSRASRADDGSLTSQTKLEDNPVENEEASTHEAASFEDKPENLPELAESSLPAGPAPEEGEGGPEPQPSADQGSAELCGSQEVDQPTSGADTGTSDASGGSRRAVSETESLDQGSEPSQVSSETEPSDPARTESVSEASTRPEGESDLECADSSCNESVTTQLSSVDTRCSSLESARFPETPAFSSQEEEDGACAAEPTSSGPAEGSQESVCTAGSLPVVQVPSGEDEGPGAESATVPDQEELGEVWQRRGSLEGAAAAAESPPQEEGSAGEAQGTCEGATAQEEGATGGSQANGHQPLRSLPSVRQDVSRYQRVDEALPPNWEARIDSHGRIFYVDHVNRTTTWQRPTAPPAPQVLQRSNSIQQMEQLNRRYQSIRRTMTNERPEENTNAIDGAGEEADFHQASADFRRENILPHSTSRSRITLLLQSPPVKFLISPEFFTVLHSNPSAYRMFTNNTCLKHMITKVRRDTHHFERYQHNRDLVGFLNMFANKQLELPRGWEMKHDHQGKAFFVDHNSRTTTFIDPRLPLQSSRPTSALVHRQHLTRQRSHSAGEVGEDSRHAGPPVLPRPSSTFNTVSRPQYQDMVPVAYNDKIVAFLRQPNIFEILQERQPDLTRNHSLREKIQFIRTEGTPGLVRLSSDADLVMLLSLFEEEIMSYVPPHALLHPSYCQSPRGSPVSSPQNSPGTQRANARAPAPYKRDFEAKLRNFYRKLETKGYGQGPGKLKLIIRRDHLLEDAFNQIMGYSRKDLQRNKLYVTFVGEEGLDYSGPSREFFFLVSRELFNPYYGLFEYSANDTYTVQISPMSAFVDNHHEWFRFSGRILGLALIHQYLLDAFFTRPFYKALLRILCDLSDLEYLDEEFHQSLQWMKDNDIHDILDLTFTVNEEVFGQITERELKPGGANIPVTEKNKKEYIERMVKWRIERGVVQQTESLVRGFYEVVDARLVSVFDARELELVIAGTAEIDLSDWRNNTEYRGGYHDNHIVIRWFWAAVERFNNEQRLRLLQFVTGTSSIPYEGFASLRGSNGPRRFCVEKWGKITALPRAHTCFNRLDLPPYPSFSMLYEKLLTAVEETSTFGLE

SEQ ID NO: 43 - protein, 34 aa, Homo sapiens
EALPPNWEARIDSHGRIFYVDHVNRTTTWQRPTA

SEQ ID NO: 44 - protein, 34 aa, Homo sapiens
LELPRGWEMKHDHQGKAFFVDHNSRTTTFIDPRL

SEQ ID NO: 45 - protein, 390 aa, Homo sapiens
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 46 - protein, 391 aa, Mus musculus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTATGPPNTSYMPGMPSGISAYPSGYPPNPSGYPGCPYPPAGPYPATTSSQYPSQPPVTTVGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 47 - protein, 391 aa, Rattus norvegicus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTAAGPPNTSYLPSMPSGISAYPSGYPPNPSGYPGCPYPPAGPYPATTSSQYPSQPPVTTAGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 48 - DNA, 3475 bp, Homo sapiens
  source 1..3475: mol_type = genomic DNA
gaattcgcggccgcgtcgaccgcttctgtggccacggcagatgaaacagaaaggctaaagagggctggagtcaggggacttctcttccaccagcttcacggtgatgatatggcatctgccagctctagccgggcaggagtggccctgccttttgagaagtctcagctcactttgaaagtggtgtccgcaaagcccaaggtgcataatcgtcaacctcgaattaactcctacgtggaggtggcggtggatggactccccagtgagaccaagaagactgggaagcgcattgggagctctgagcttctctggaatgagatcatcattttgaatgtcacggcacagagtcatttagatttaaaggtctggagctgccataccttgagaaatgaactgctaggcaccgcatctgtcaacctctccaacgtcttgaagaacaatgggggcaaaatggagaacatgcagctgaccctgaacctgcagacggagaacaaaggcagcgttgtctcaggcggaaaactgacaattttcctggacgggccaactgttgatctgggaaatgtgcctaatggcagtgccctgacagatggatcacagctgccttcgagagactccagtggaacagcagtagctccagagaaccggcaccagccccccagcacaaactgctttggtggaagatcccggacgcacagacattcgggtgcttcagccagaacaaccccagcaaccggcgagcaaagccccggtgctcggagccggcaccgccagcccgtcaagaactcaggccacagtggcttggccaatggcacagtgaatgatgaacccacaacagccactgatcccgaagaaccttccgttgttggtgtgacgtccccacctgctgcacccttgagtgtgaccccgaatcccaacacgacttctctccctgccccagccacaccggctgaaggagaggaacccagcacttcgggtacacagcagctcccagcggctgcccaggcccccgacgctctgcctgctggatgggaacagcgagagctgcccaacggacgtgtctattatgttgaccacaataccaagaccaccacctgggagcggccccttcctccaggctgggaaaaacgcacagatccccgaggcaggttttactatgtggatcacaatactcggaccaccacctggcagcgtccgaccgcggagtacgtgcgcaactatgagcagtggcagtcgcagcggaatcagctccagggggccatgcagcacttcagccaaagattcctataccagttttggagtgcttcgactgaccatgatcccctgggccccctccctcctggttgggagaaaagacaggacaatggacgggtgtattacgtgaaccataacactcgcacgacccagtgggaggatccccggacccaggggatgatccaggaaccagctttgcccccaggatgggagatgaaatacaccagcgagggggtgcgatactttgtggaccacaatacccgcaccaccacctttaaggatcctcgcccggggtttgagtcggggacgaagcaaggttcccctggtgcttatgaccgcagttttcggtggaagtatcaccagttccgtttcctctgccattcaaatgccctacctagccacgtgaagatcagcgtttccaggcagacgcttttcgaagattccttccaacagatcatgaacatgaaaccctatgacctgcgccgccggctttacatcatcatgcgtggcgaggagggcctggactatgggggcatcgccagagagtggtttttcctcctgtctcacgaggtgctcaaccctatgtattgtttatttgaatatgccggaaagaacaattactgcctgcagatcaaccccgcctcctccatcaacccggaccacctcacctactttcgctttataggcagattcatcgccatggcgctgtaccatggaaagttcatcgacacgggcttcaccctccctttctacaagcggatgctcaataagagaccaaccctgaaagacctggagtccattgaccctgagttctacaactccattgtctggatcaaagagaacaacctggaagaatgtggcctggagctgtacttcatccaggacatggagatactgggcaaggtgacgacccacgagctgaaggagggcggcgagagcatccgggtcacggaggagaacaaggaagagtacatcatgctgctgactgactggcgtttcacccgaggcgtggaagagcagaccaaagccttcctggatggcttcaacgaggtggccccgctggagtggctgcgctactttgacgagaaagagctggagctgatgctgtgcggcatgcaggagatagacatgagcgactggcagaagagcaccatctaccggcactacaccaagaacagcaagcagatccagtggttctggcaggtggtgaaggagatggacaacgagaagaggatccggctgctgcagtttgtcaccggtacctgccgcctgcccgtcgggggatttgccgaactcatcggtagcaacggaccacagaagttttgcattgacaaagttggcaaggaaacctggctgcccagaagccacacctgcttcaaccgtctggatcttccaccctacaagagctacgaacagctgagagagaagctgctgtatgccattgaggagaccgagggctttggacaggagtaaccgaggccgcccctcccacgccccccagcgcacatgtagtcctgagtcctccctgcctgagaggccactggccccgcagcccttgggaggcccccgtggatgtggccctgtgtgggaccacactgtcatctcgctgctggcagaaaagcctgatcccaggaggccctgcagttcccccgacccgcggatggcagtctggaataaagccccctagttgcctttggccccacctttgcaaagttccagagggctgaccctctctgcaaaactctcccctgtcctctagaccccaccctgggtgtatgtgagtgtgcaagggaaggtgttgcatccccaggggctgccgcagaggccggagacctcctggactagttcggcgaggagactggccactgggggtggctgttcgggactgagagcgccaagggtctttgccagcaaaggaggttctgcctgtaattgagcctctctgatgatggagatgaagtgaaggtctgagggacgggccctggggctaggccatctctgcctgcctccctagcaggcgccagcggtggaggctgagtcgcaggacacatgccggccagttaattcattctcagcaaatgaaggtttgtctaagctgcctgggtatccacgggacaaaaacagcaaactccctccagactttgtccatgttataaacttgaaagttggttgttgtttgttaggtttgccaggtttttttgtttacgcctgctgtcactttcctgtc

SEQ ID NO: 49 - DNA, 3475 bp, Homo sapiens; identical to SEQ ID NO: 48
  source 1..3475: mol_type = genomic DNA

SEQ ID NO: 50 - DNA, 2914 bp, Homo sapiens
  source 1..2914: mol_type = genomic DNA
acagttgcctgccctgggcgggggcgagcgcgtccggtttgctggaagcgttcggaaatggcaacttgcgcggtggaggtgttcgggctcctggaggacgaggaaaattcacgaattgtgagagtaagagttatagccggaataggccttgccaagaaggatatattgggagctagtgatccttacgtgagagtgacgttatatgacccaatgaatggagttcttacaagtgtgcaaacaaaaaccattaaaaagagtttgaatccaaagtggaatgaagaaatattattcagagttcatcctcagcagcaccggcttctttttgaagtgtttgacgaaaaccgattgacaagagatgatttcctaggtcaagtggatgttccactttatccattaccgacagaaaatccaagattggagagaccatatacatttaaggattttgttcttcatccaagaagtcacaaatcaagagttaaaggttatctgagactaaaaatgacttatttacctaaaaccagtggctcagaagatgataatgcagaacaggctgaggaattagagcctggctgggttgttttggaccaaccagatgctgcttgccatttgcagcaacaacaagaaccttctcctctacctccagggtgggaagagaggcaggatatccttggaaggacctattatgtaaaccatgaatctagaagaacacagtggaaaagaccaacccctcaggacaacctaacagatgctgagaatggcaacattcaactgcaagcacaacgtgcatttaccaccaggcggcagatatccgaggaaacagaaagtgttgacaaccaagagtcttccgagaactgggaaattataagagaagatgaagccaccatgtatagcagccaggccttcccatcacctccaccgtcaagtaacttggatgttccaactcatcttgcagaagaattgaatgccagactcaccatttttggaaattcagccgtgagccagccagcatcgagctcaaatcattccagcagaagaggcagcttacaagcctatacttttgaggaacaacctacacttcctgtgcttttgcctacttcatctggattaccaccaggttgggaagaaaaacaagatgaaagaggaagatcatattatgtagatcacaattccagaacgactacttggacaaagcccactgtacaggccacagtggagaccagtcagctgacctcaagccagagttctgcaggccctcaatcacaagcctccaccagtgattcaggccagcaggtgacccagccatctgaaattgagcaaggattccttcctaaaggctgggaagtccggcatgcaccaaatgggaggcctttctttattgaccacaacactaaaaccaccacctgggaagatccaagattgaaaattccagcccatctgagaggaaagacatcacttgatacttccaatgatctagggcctttacctccaggatgggaagagagaactcacacagatggaagaatcttctacataaatcacaatataaaaagaacacaatgggaagatcctcggttggagaatgtagcaataactggaccagcagtgccctactccagggattacaaaagaaagtatgagttcttccgaagaaagttgaagaagcagaatgacattccaaacaaatttgaaatgaaacttcgccgagcaactgttcttgaagactcttaccggagaattatgggtgtcaagagagcagacttcctgaaggctcgactgtggattgagtttgatggtgaaaagggattggattatggaggagttgccagagaatggttcttcctgatctcaaaggaaatgtttaacccttattatgggttgtttgaatattctgctacggacaattataccctacagataaatccaaactctggattgtgtaacgaagatcacctctcttacttcaagtttattggtcgggtagctggaatggcagtttatcatggcaaactgttggatggttttttcatccgcccattttacaagatgatgcttcacaaaccaataacccttcatgatatggaatctgtggatagtgaatattacaattccctaagatggattcttgaaaatgacccaacagaattggacctcaggtttatcatagatgaagaactttttggacagacacatcaacatgagctgaaaaatggtggatcagaaatagttgtcaccaataagaacaaaaaggaatatatttatcttgtaatacaatggcgatttgtaaaccgaatccagaagcaaatggctgcttttaaagagggattctttgaactaataccacaggatctcatcaaaatttttgatgaaaatgaactagagcttcttatgtgtggaccgggagatgttgatgtgaatgactggagggaacatacaaagtataaaaatggctacagtgcaaatcatcaggttatacagtggttttggaaggctgttttaatgatggattcagaaaaaagaataagattacttcagtttgtcactggcacatctcgggtgcctatgaatggatttgctgaactatacggttcaaatggaccacagtcatttacagttgaacagtggggtactcctgaaaagctgccaagagctcatacctgttttaatcgcctggacttgccaccttatgaatcatttgaagaattatgggataaacttcagatggcaattgaaaacacccagggctttgatggagttgattagattacaaataacaatctgtagtgtttttactgccatagttttataaccaaaatcttgacttaaaattttccggggaactactaaaatgtggccactgagtcttcccagatcttgaagaaaatcatataaaaagcatttgaagaaatagtacgac

SEQ ID NO: 51 - DNA, 2868 bp, Homo sapiens
  source 1..2868: mol_type = genomic DNA
atggcgaccgggctcggggagccggtctatggactttccgaagacgagggagagtcccgtattctcagagtaaaagttgtttctggaattgatctcgccaaaaaggacatctttggagccagtgatccgtatgtgaaactttcattgtacgtagcggatgagaatagagaacttgctttggtccagacaaaaacaattaaaaagacactgaacccaaaatggaatgaagaattttatttcagggtaaacccatctaatcacagactcctatttgaagtatttgacgaaaatagactgacacgagacgacttcctgggccaggtggacgtgccccttagtcaccttccgacagaagatccaaccatggagcgaccctatacatttaaggactttctcctcagaccaagaagtcataagtctcgagttaagggatttttgcgattgaaaatggcctatatgccaaaaaatggaggtcaagatgaagaaaacagtgaccagagggatgacatggagcatggatgggaagttgttgactcaaatgactcggcttctcagcaccaagaggaacttcctcctcctcctctgcctcccgggtgggaagaaaaagtggacaatttaggccgaacttactatgtcaaccacaacaaccggaccactcagtggcacagaccaagcctgatggacgtgtcctcggagtcggacaataacatcagacagatcaaccaggaggcagcacaccggcgcttccgctcccgcaggcacatcagcgaagacttggagcccgagccctcggagggcggggatgtccccgagccttgggagaccatttcagaggaagtgaatatcgctggagactctctcggtctggctctgcccccaccaccggcctccccaggatctcggaccagccctcaggagctgtcagaggaactaagcagaaggcttcagatcactccagactccaatggggaacagttcagctctttgattcaaagagaaccctcctcaaggttgaggtcatgcagtgtcaccgacgcagttgcagaacagggccatctaccaccgccatcagtggcctatgtacataccacgccgggtctgccttcaggctgggaagaaagaaaagatgctaaggggcgcacatactatgtcaatcataacaatcgaaccacaacttggactcgacctatcatgcagcttgcagaagatggtgcgtccggatcagccacaaacagtaacaaccatctaatcgagcctcagatccgccggcctcgtagcctcagctcgccaacagtaactttatctgccccgctggagggtgccaaggactcacccgtacgtcgggctgtgaaagacaccctttccaacccacagtccccacagccatcaccttacaactcccccaaaccacaacacaaagtcacacagagcttcttgccacccggctgggaaatgaggatagcgccaaacggccggcccttcttcattgatcataacacaaagactacaacctgggaagatccacgtttgaaatttccagtacatatgcggtcaaagacatctttaaaccccaatgaccttggcccccttcctcctggctgggaagaaagaattcacttggatggccgaacgttttatattgatcataatagcaaaattactcagtgggaagacccaagactgcagaacccagctattactggtccggctgtcccttactccagagaatttaagcagaaatatgactacttcaggaagaaattaaagaaacctgctgatatccccaataggtttgaaatgaaacttcacagaaataacatatttgaagagtcctatcggagaattatgtccgtgaaaagaccagatgtcctaaaagctagactgtggattgagtttgaatcagagaaaggtcttgactatgggggtgtggccagagaatggttcttcttactgtccaaagagatgttcaacccctactacggcctctttgagtactctgccacggacaactacacccttcagatcaaccctaattcaggcctctgtaatgaggatcatttgtcctacttcacttttattggaagagttgctggtctggccgtatttcatgggaagctcttagatggtttcttcattagaccattttacaagatgatgttgggaaagcagataaccctgaatgacatggaatctgtggatagtgaatattacaactctttgaaatggatcctggagaatgaccctactgagctggacctcatgttctgcatagacgaagaaaactttggacagacatatcaagtggatttgaagcccaatgggtcagaaataatggtcacaaatgaaaacaaaagggaatatatcgacttagtcatccagtggagatttgtgaacagggtccagaagcagatgaacgccttcttggagggattcacagaactacttcctattgatttgattaaaatttttgatgaaaatgagctggagttgctcatgtgcggcctcggtgatgtggatgtgaatgactggagacagcattctatttacaagaacggctactgcccaaaccaccccgtcattcagtggttctggaaggctgtgctactcatggacgccgaaaagcgtatccggttactgcagtttgtcacagggacatcgcgagtacctatgaatggatttgccgaactttatggttccaatggtcctcagctgtttacaatagagcaatggggcagtcctgagaaactgcccagagctcacacatgctttaatcgccttgacttacctccatatgaaacctttgaagatttacgagagaaacttctcatggccgtggaaaatgctcaaggatttgaaggggtggattaa

SEQ ID NO: 52 - DNA, 2274 bp, Homo sapiens
  source 1..2274: mol_type = genomic DNA
atgtcgaaccccgggacacgcaggaacggctccagcatcaagatccgtctgacagtgttatgtgccaagaaccttgcaaagaaagacttcttcaggctccctgacccttttgcaaagattgtcgtggatgggtctgggcagtgccactcaaccgacactgtgaaaaacacattggacccaaagtggaaccagcactatgatctatatgttgggaaaacggattcgataaccattagcgtgtggaaccataagaaaattcacaagaaacagggagctggcttcctgggctgtgtgcggctgctctccaatgccatcagcagattaaaagataccggataccagcgtttggatctatgcaaactaaacccctcagatactgatgcagttcgtggccagatagtggtcagtttacagacacgagacagaataggaaccggcggctcggtggtggactgcagaggactgttagaaaatgaaggaacggtgtatgaagactccgggcctgggaggccgctcagctgcttcatggaggaaccagccccttacacagatagcaccggtgctgctgctggaggagggaattgcaggttcgtggagtccccaagtcaagatcaaagacttcaggcacagcggcttcgaaaccctgatgtgcgaggttcactacagacgccccagaaccgaccacacggccaccagtccccggaactgcccgaaggctacgaacaaagaacaacagtccagggccaagtttactttttgcatacacagactggagttagcacgtggcacgaccccaggataccaagtccctcggggaccattcctgggggagatgcagcttttctatacgaattccttctacaaggccatacatctgagcccagagaccttaacagtgtgaactgtgatgaacttggaccactgccgccaggctgggaagtcagaagtacagtttctgggaggatatattttgtagatcataataaccgaacaacccagtttacagacccaaggttacaccacatcatgaatcaccagtgccaactcaaggagcccagccagccgctgccactgcccagtgagggctctctggaggacgaggagcttcctgcccagagatacgaaagagatctagtccagaagctgaaagtcctcagacacgaactgtcgcttcagcagccccaagctggtcattgccgcatcgaagtgtccagagaagaaatctttgaggagtcttaccgccagataatgaagatgcgaccgaaagacttgaaaaaacggctgatggtgaaattccgtggggaagaaggtttggattacggtggtgtggccagggagtggctttacttgctgtgccatgaaatgctgaatccttattacgggctcttccagtattctacggacaatatttacatgttgcaaataaatccggattcttcaatcaaccccgaccacttgtcttatttccactttgtggggcggatcatggggctggctgtgttccatggacactacatcaacgggggcttcacagtgcccttctacaagcagctgctggggaagcccatccagctctcagatctggaatctgtggacccagagctgcataagagcttggtgtggatcctagagaacgacatcacgcctgtactggaccacaccttctgcgtggaacacaacgccttcgggcggatcctgcagcatgaactgaaacccaatggcagaaatgtgccagtcacagaggagaataagaaagaatacgtccggttgtatgtaaactggaggtttatgagaggaatcgaagcccagttcttagctctgcagaaggggttcaatgagctcatccctcaacatctgctgaagccttttgaccagaaggaactggagctgatcataggcggcctggataaaatagacttgaacgactggaagtcgaacacgcggctgaagcactgtgtggccgacagcaacatcgtgcggtggttctggcaagcggtggagacgttcgatgaagaaaggagggccaggctcctgcagtttgtgactgggtccacgcgagtcccgctccaaggcttcaaggctttgcaaggttctacaggcgcggcagggccccggctgttcaccatccacctgatagacgcgaacacagacaaccttccgaaggcccatacctgctttaaccggatcgacattccaccatatgagtcctatgagaagctctacgagaagctgctgacagccgtggaggagacctgcgggtttgctgtggagtga

SEQ ID NO: 53 - DNA, 2247 bp, Homo sapiens
  source 1..2247: mol_type = genomic DNA
atgtctaaccccggacgccggaggaacgggcccgtcaagctgcgcctgacagtactctgtgcaaaaaacctggtgaaaaaggattttttccgacttcctgatccatttgctaaggtggtggttgatggatctgggcaatgccattctacagatactgtgaagaatacgcttgatccaaagtggaatcagcattatgacctgtatattggaaagtctgattcagttacgatcagtgtatggaatcacaagaagatccataagaaacaaggtgctggatttctcggttgtgttcgtcttctttccaatgccatcaaccgcctcaaagacactggttatcagaggttggatttatgcaaactcgggccaaatgacaatgatacagttagaggacagatagtagtaagtcttcagtccagagaccgaataggcacaggaggacaagttgtggactgcagtcgtttatttgataacgatttaccagacggctgggaagaaaggagaaccgcctctggaagaatccagtatctaaaccatataacaagaactacgcaatgggagcgcccaacacgaccggcatccgaatattctagccctggcagacctcttagctgctttgttgatgagaacactccaattagtggaacaaatggtgcaacatgtggacagtcttcagatcccaggctggcagagaggagagtcaggtcacaacgacatagaaattacatgagcagaacacatttacatactcctccagacctaccagaaggctatgaacagaggacaacgcaacaaggccaggtgtatttcttacatacacagactggtgtgagcacatggcatgatccaagagtgcccagggatcttagcaacatcaattgtgaagagcttggtccattgcctcctggatgggagatccgtaatacggcaacaggcagagtttatttcgttgaccataacaacagaacaacacaatttacagatcctcggctgtctgctaacttgcatttagttttaaatcggcagaaccaattgaaagaccaacagcaacagcaagtggtatcgttatgtcctgatgacacagaatgcctgacagtcccaaggtacaagcgagacctggttcagaaactaaaaattttgcggcaagaactttcccaacaacagcctcaggcaggtcattgccgcattgaggtttccagggaagagatttttgaggaatcatatcgacaggtcatgaaaatgagaccaaaagatctctggaagcgattaatgataaaatttcgtggagaagaaggccttgactatggaggcgttgccagggaatggttgtatctcttgtcacatgaaatgttgaatccatactatggcctcttccagtattcaagagatgatatttatacattgcagatcaatcctgattctgcagttaatccggaacatttatcctatttccactttgttggacgaataatgggaatggctgtgtttcatggacattatattgatggtggtttcacattgcctttttataagcaattgcttgggaagtcaattaccttggatgacatggagttagtagatccggatcttcacaacagtttagtgtggatacttgagaatgatattacaggtgttttggaccataccttctgtgttgaacataatgcatatggtgaaattattcagcatgaacttaaaccaaatggcaaaagtatccctgttaatgaagaaaataaaaaagaatatgtcaggctctatgtgaactggagatttttacgaggcattgaggctcaattcttggctctgcagaaaggatttaatgaagtaattccacaacatctgctgaagacatttgatgagaaggagttagagctcattatttgtggacttggaaagatagatgttaatgactggaaggtaaacacccggttaaaacactgtacaccagacagcaacattgtcaaatggttctggaaagctgtggagttttttgatgaagagcgacgagcaagattgcttcagtttgtgacaggatcctctcgagtgcctctgcagggcttcaaagcattgcaaggtgctgcaggcccgagactctttaccatacaccagattgatgcctgcactaacaacctgccgaaagcccacacttgcttcaatcgaatagacattccaccctatgaaagctatgaaaagctatatgaaaagctgctaacagccattgaagaaacatgtggatttgctgtggaatga

SEQ ID NO: 54 - DNA, 2869 bp, Homo sapiens
  source 1..2869: mol_type = genomic DNA
ggagtcgccgccgccccgagttccggtaccatgcatttcacggtggccttgtggagacaacgccttaacccaaggaagtgactcaaactgtgagaactccaggttttccaacctattggtggtatgtctgacagtggatcacaacttggttcaatgggtagcctcaccatgaaatcacagcttcagatcactgtcatctcagcaaaacttaaggaaaataagaagaattggtttggaccaagtccttacgtagaggtcacagtagatggacagtcaaagaagacagaaaaatgcaacaacacaaacagtcccaagtggaagcaaccccttacagttatcgttacccctgtgagtaaattacattttcgtgtgtggagtcaccagacactgaaatctgatgttttgttgggaactgctgcattagatatttatgaaacattaaagtcaaacaatatgaaacttgaagaagtagttgtgactttgcagcttggaggtgacaaagagccaacagagacaataggagacttgtcaatttgtcttgatgggctacagttagagtctgaagttgttaccaatggtgaaactacatgttcagaaagtgcttctcagaatgatgatggctccagatccaaggatgaaacaagagtgagcacaaatggatcagatgaccctgaagatgcaggagctggtgaaaataggagagtcagtgggaataattctccatcactctcaaatggtggttttaaaccttctagacctccaagaccttcacgaccaccaccacccaccccacgtagaccagcatctgtcaatggttcaccatctgccacttctgaaagtgatgggtctagtacaggctctctgccgccgacaaatacaaatacaaatacatctgaaggagcaacatctggattaataattcctcttactatatctggaggctcaggccctaggccattaaatcctgtaactcaagctcccttgccacctggttgggagcagagagtggaccagcacgggcgagtttactatgtagatcatgttgagaaaagaacaacatgggatagaccagaacctctacctcctggctgggaacggcgggttgacaacatgggacgtatttattatgttgaccatttcacaagaacaacaacgtggcagaggccaacactggaatccgtccggaactatgaacaatggcagctacagcgtagtcagcttcaaggagcaatgcagcagtttaaccagagattcatttatgggaatcaagatttatttgctacatcacaaagtaaagaatttgatcctcttggtccattgccacctggatgggagaagagaacagacagcaatggcagagtatatttcgtcaaccacaacacacgaattacacaatgggaagaccccagaagtcaaggtcaattaaatgaaaagcccttacctgaaggttgggaaatgagattcacagtggatggaattccatattttgtggaccacaatagaagaactaccacctatatagatccccgcacaggaaaatctgccctagacaatggacctcagatagcctatgttcgggacttcaaagcaaaggttcagtatttccggttctggtgtcagcaactggccatgccacagcacataaagattacagtgacaagaaaaacattgtttgaggattcctttcaacagataatgagcttcagtccccaagatctgcgaagacgtttgtgggtgatttttccaggagaagaaggtttagattatggaggtgtagcaagagaatggttctttcttttgtcacatgaagtgttgaacccaatgtattgcctgtttgaatatgcagggaaggataactactgcttgcagataaaccccgcttcttacatcaatccagatcacctgaaatattttcgttttattggcagatttattgccatggctctgttccatgggaaattcatagacacgggtttttctttaccattctataagcgtatcttgaacaaaccagttggactcaaggatttagaatctattgatccagaattttacaattctctcatctgggttaaggaaaacaatattgaggaatgtgatttggaaatgtacttctccgttgacaaagaaattctaggtgaaattaagagtcatgatctgaaacctaatggtggcaatattcttgtaacagaagaaaataaagaggaatacatcagaatggtagctgagtggaggttgtctcgaggtgttgaagaacagacacaagctttctttgaaggctttaatgaaattcttccccagcaatatttgcaatactttgatgcaaaggaattagaggtccttttatgtggaatgcaagagattgatttgaatgactggcaaagacatgccatctaccgtcattatgcaaggaccagcaaacaaatcatgtggttttggcagtttgttaaagaaattgataatgagaagagaatgagacttctgcagtttgttactggaacctgccgattgccagtaggaggatttgctgatctcatggggagcaatggaccacagaaattctgcattgaaaaagttgggaaagaaaattggctacccagaagtcatacctgttttaatcgcctggacctgccaccatacaagagctatgagcaactgaaggaaaagctgttgtttgccatagaagaaacagaaggatttggacaagagtaacttctgagaacttgcaccatgaatgggcaagaacttatttgcaatgtttgtccttctctgcctgttgcacatcttgtaaaattggacaatggctctttagagagttatctgagtgtaagtaaattaatgttctcatttaaaaaaaaaaaaaaaaaaa

SEQ ID NO: 55 - DNA, 5169 bp, Homo sapiens
  source 1..5169: mol_type = genomic DNA
gcgcatcaggcgctgttgttggagccggaacaccgtgcgactctgaccgaaccggccccctcctcgcgcacacactcgccgagccgcgcgcgcccctccgccgtgacagtggccgtggcctccgctctctcggggcacccggcagccagagcgcagcgagagcgggcggtcgccagggtcccctccccagccagtcccaggcgcccggtgcactatgcggggcacgtgcgccccccagctctaatctgcgcgctgacaggagcatgatctgtgcccaggccagggctgccaaggaattgatgcgcgtacacgtggtgggtcattatgctgctacacctgtgtagtgtgaagaatctgtaccagaacaggtttttaggcctggccgccatggcgtctccttctagaaactcccagagccgacgccggtgcaaggagccgctccgatacagctacaaccccgaccagttccacaacatggacctcaggggcggcccccacgatggcgtcaccattccccgctccaccagcgacactgacctggtcacctcggacagccgctccacgctcatggtcagcagctcctactattccatcgggcactctcaggacctggtcatccactgggacataaaggaggaagtggacgctggggactggattggcatgtacctcattgatgaggtcttgtccgaaaactttctggactataaaaaccgtggagtcaatggttctcatcggggccagatcatctggaagatcgatgccagctcgtactttgtggaacctgaaactaagatctgcttcaaatactaccatggagtgagtggggccctgcgagcaaccacccccagtgtcacggtcaaaaactcggcagctcctatttttaaaagcattggtgctgatgagaccgtccaaggacaaggaagtcggaggctgatcagcttctctctctcagatttccaagccatggggttgaagaaagggatgtttttcaacccagacccttatctgaagatttccattcagcctgggaaacacagcatcttccccgccctccctcaccatggacaggagaggagatccaagatcataggcaacaccgtgaaccccatctggcaggccgagcaattcagttttgtgtccttgcccactgacgtgctggaaattgaggtgaaggacaagtttgccaagagccgccccatcatcaagcgcttcttgggaaagctgtcgatgcccgttcaaagactcctggagagacacgccataggggatagggtggtcagctacacacttggccgcaggcttccaacagatcatgtgagtggacagctgcaattccgatttgagatcacttcctccatccacccagatgatgaggagatttccctgagtaccgagcctgagtcagcccaaattcaggacagccccatgaacaacctgatggaaagcggcagtggggaacctcggtctgaggcaccagagtcctctgagagctggaagccagagcagctgggtgagggcagtgtccccgatggtccagggaaccaaagcatagagctttccagaccagctgaggaagcagcagtcatcacggaggcaggagaccagggcatggtctctgtgggacctgaaggggctggggagctcctggcccaggtgcaaaaggacatccagcctgcccccagtgcagaagagctggccgagcagctggacctgggtgaggaggcatcagcactgctgctggaagacggtgaagccccagccagcaccaaggaggagcccttggaggaggaagcaacgacccagagccgggctggaagggaagaagaggagaaggagcaggaggaggagggagatgtgtctaccctggagcagggagagggcaggctgcagctgcgggcctcggtgaagagaaaaagcaggccctgctccttgcctgtgtccgagctggagacggtgatcgcgtcagcctgcggggaccccgagaccccgcggacacactacatccgcatccacaccctgctgcacagcatgccctccgcccagggcggcagcgcggcagaggaggaggacggcgcggaggaggagtccaccctcaaggactcctcggagaaggatgggctcagcgaggtggacacggtggccgctgacccgtctgccctggaagaggacagagaagagcccgagggggctactccaggcacggcgcaccctggccactccgggggccacttccccagcctggccaatggcgcggcccaggatggcgacacgcaccccagcaccgggagcgagagcgactccagccccaggcaaggcggggaccacagttgcgagggctgtgacgcgtcctgctgcagcccctcgtgctacagctcctcgtgctacagcacgtcctgctacagcagctcgtgctacagcgcctcgtgctacagcccctcctgctacaacggcaacaggttcgccagccacacgcgcttctcctccgtggacagcgccaagatctccgagagcacggtcttctcctcgcaagacgacgaggaggaggagaacagcgcgttcgagtcggtacccgactccatgcagagccctgagctggacccggagtccacgaacggcgctgggccgtggcaagacgagctggccgcccctagcgggcacgtggaaagaagcccggaaggtctggaatcccccgtggcaggtccaagcaatcggagagaagactgggaagctcgaattgacagccacgggcgggtcttttatgtggaccacgtgaaccgcacaaccacctggcagcgtccgacggcagcagccaccccggatggcatgcggagatcggggtccatccagcagatggagcaactcaacaggcggtatcaaaacattcagcgaaccattgcaacagagaggtccgaagaagattctggcagccaaagctgcgagcaagccccagcaggaggaggcggaggtggagggagtgactcagaagccgaatcttcccagtccagcttagatctaaggagagaggggtcactttctccagtgaactcacaaaaaatcaccttgctgctgcagtccccagcggtcaagttcatcaccaaccccgagttcttcactgtgctacacgccaattatagtgcctaccgagtcttcaccagtagcacctgcttaaagcacatgattctgaaagtccgacgggatgctcgcaattttgaacgctaccagcacaaccgggacttggtgaatttcatcaacatgttcgcagacactcggctggaactgccccggggctgggagatcaaaacggaccagcagggaaagtcttttttcgtggaccacaacagtcgagctaccactttcattgacccccgaatccctcttcagaacggtcgtcttcccaatcatctaactcaccgacagcacctccagaggctccgaagttacagcgccggagaggcctcagaagtttctagaaacagaggagcctctttactggccaggccaggacacagcttagtagctgctattcgaagccaacatcaacatgagtcattgccactggcatataatgacaagattgtggcatttcttcgccagccaaacatttttgaaatgctgcaagagcgtcagccaagcttagcaagaaaccacacactcagggagaaaatccattacattcggactgagggtaatcacgggcttgagaagttgtcctgtgatgcggatctggtcattttgctgagtctctttgaagaagagattatgtcctacgtccccctgcaggctgccttccaccctgggtatagcttctctccccgatgttcaccctgttcttcacctcagaactccccaggtttacagagagccagtgcaagagccccttccccctaccgaagagactttgaggccaagctccgcaatttctacagaaaactggaagccaaaggatttggtcagggtccggggaaaattaagctcattattcgccgggatcatttgttggagggaaccttcaatcaggtgatggcctattcgcggaaagagctccagcgaaacaagctctacgtcacctttgttggagaggagggcctggactacagtggcccctcgcgggagttcttcttccttctgtctcaggagctcttcaacccttactatggactctttgagtactcggcaaatgatacttacacggtgcagatcagccccatgtccgcatttgtagaaaaccatcttgagtggttcaggtttagcggtcgcatcctgggtctggctctgatccatcagtaccttcttgacgctttcttcacgaggcccttctacaaggcactcctgagactgccctgtgatttgagtgacctggaatatttggatgaggaattccaccagagtttgcagtggatgaaggacaacaacatcacagacatcttagacctcactttcactgttaatgaagaggtttttggacaggtcacggaaagggagttgaagtctggaggagccaacacacaggtgacggagaaaaacaagaaggagtacatcgagcgcatggtgaagtggcgggtggagcgcggcgtggtacagcagaccgaggcgctggtgcgcggcttctacgaggttgtagactcgaggctggtgtccgtgtttgatgccagggagctggagctggtgatagctggcaccgcggaaatcgacctaaatgactggcggaataacactgagtaccggggaggttaccacgatgggcatcttgtgatccgctggttctgggctgcggtggagcgcttcaataatgagcagaggctgagattactgcagtttgtcacgggaacatccagcgtgccctacgaaggcttcgcagccctccgtgggagcaatgggcttcggcgcttctgcatagagaaatgggggaaaattacttctctccccagggcacacacatgcttcaaccgactggatcttccaccgtatccctcgtactccatgttgtatgaaaagctgttaacagcagtagaggaaaccagcacctttggacttgagtgaggacatggaacctcgcctgacattttcctggccagtgacatcacccttcctgggatgatccccttttccctttcccttaatcaactctcctttgattttggtattccatgatttttattttcaaac

SEQ ID NO: 56 - DNA, 2715 bp, Homo sapiens
  source 1..2715: mol_type = genomic DNA
agagttccatcagagcctgcagtggatgaaagacaatgatatccatgacatcctagacctcacgttcactgtgaacgaagaagtatttgggcagataactgaacgagaattaaagccagggggtgccaatatcccagttacagagaagaacaagaaggagtacatcgagaggatggtgaagtggaggattgagaggggtgttgtacagcaaacagagagcttagtgcgtggcttctatgaggtggtggatgccaggctggtatctgtttttgatgcaagagaactggaattggtcatcgcaggcacagctgaaatagacctaagtgattggagaaacaacacagaatatagaggaggataccatgacaatcatattgtaattcggtggttctgggctgcagtggaaagattcaacaatgaacaacgactaaggttgttacagtttgttacaggcacatccagcattccctatgaaggatttgcttcactccgagggagtaacggcccaagaagattctgtgtggagaaatgggggaaaatcactgctcttcccagagcgcatacatgttttaaccgtctggatctgcctccctacccatccttttccatgctttatgaaaaactgttgacagcagttgaagaaaccagtacttttggacttgagtgacctggaagctgaatgcccatctctgtggacaggcagtttcagaagctgccttctagaagaatgattgaacattggaagtttcaagaggatgcttcctttaggataaagctacgtgctgttgttttccaggaacaagtgctctgtcacatttggggactggagatgagtcctcttggaaggatttgggtgagcttgatgcccagggaacaacccaaccgtctttcaatcaacagttcttgactgccaaactttttccatttgttatgttccaagacaaagatgaacccatacatgatcagctccacggtaatttttagggactcaggagaatcttgaaacttacccttgaacgtggttcaagccaaactggcagcatttggcccaatctccaaattagagcaagttaaataatataataaaagtaaatatatttcctgaaagtacattcatttaagccctaagttataacagaatattcatttcttgcttatgagtgcctgcatggtgtgcaccataggtttccgctttcatgggacatgagtgaaaatgaaaccaagtcaatatgaggtacctttacagatttgcaataagatggtctgtgacaatgtatatgcaagtggtatgtgtgtaattatggctaaagacaaaccattattcagtgaattactaatgacagattttatgctttataatgcatgaaaacaattttaaaataactagcaattaatcacagcatatcaggaaaaagtacacagtgagttctgtttattttttgtaggctcattatgtttatgttctttaagatgtatataagaacctacttatcatgctgtatgtatcactcattccattttcatgttccatgcatactcgggcatcatgctaatatgtatccttttaagcactctcaaggaaacaaaagggccttttatttttataaaggtaaaaaaaattccccaaatattttgcactgaatgtaccaaaggtgaagggacattacaatatgactaacagcaactccatcacttgagaagtataatagaaaatagcttctaaatcaaacttccttcacagtgccgtgtctaccactacaaggactgtgcatctaagtaataattttttaagattcactatatgtgatagtatgatatgcatttatttaaaatgcattagactctcttccatccatcaaatactttacaggatggcatttaatacagatatttcgtatttcccccactgctttttatttgtacagcatcattaaacactaagctcagttaaggagccatcagcaacactgaagagatcagtagtaagaattccattttccctcatcagtgaagacaccacaaattgaaactcagaactatatttctaagcctgcattttcactgatgcataattttcttattaatattaagagacagtttttctatggcatctccaaaactgcatgacatcactagtcttacttctgcttaattttatgagaaggtattcttcattttaattgcttttgggattactccacatctttgtttatttcttgactaatcagattttcaatagagtgaagttaaattgggggtcataaaagcattggattgacatatggtttgccagcctatgggtttacaggcattgcccaaacatttctttgagatctatatttataagcagccatggaattcctattatgggatgttggcaatcttacattttatagaggtcatatgcatagttttcataggtgttttgtaagaactgattgctctcctgtgagttaagctatgtttactactgggaccctcaagaggaataccacttatgttacactcctgcactaaaggcacgtactgcagtgtgaagaaatgttctgaaaaagggttatagaaatctggaaataagaaaggaagagctctctgtattctataattggaagagaaaaaaagaaaaacttttaactggaaatgttagtttgtacttattgatcatgaatacaagtatatatttaattttgcaaaaaaaaaaaaaaaaaaaaaaag

SEQ ID NO: 57 - DNA, 2526 bp, Synthetic construct
  source 1..2526: mol_type = other DNA
atgaagaagatcaacgagggcctgctggacagcaagatcctgagcgccttcaacaccgtgatcgccctgctgggcagcatcgtgatcattgtgatgaacatcatgatcatccagaactacaccagaagcaccgacaaccaggccgtgatcaaggacgctctccaggggatccagcagcagatcaagggcctggccgacaagatcggcaccgagatcggccccaaggtgtccctgatcgacaccagcagcaccatcaccatccccgccaacatcggcctgctggggtccaagatcagccagagcaccgccagcatcaacgagaacgtgaacgagaagtgcaagttcaccctgccccccctgaagatccacgagtgcaacatcagctgccccaaccccctgcccttccgggagtaccggccccagaccgagggcgtgagcaacctggtcggcctgcccaacaacatctgcctgcagaaaaccagcaaccagatcctgaagcccaagctcatttcctacaccctgcccgtggtgggccagagcggcacctgcatcaccgaccccctgctggccatggacgagggctacttcgcctacagccacctggaacggatcggcagctgcagcaggggcgtgtccaagcagcggatcatcggcgtgggcgaggtgctggaccggggcgacgaggtgcccagcctgttcatgaccaacgtgtggaccccccccaaccccaacaccgtgtaccactgcagcgccgtgtacaacaacgagttctactacgtgctgtgcgccgtgagcaccgtgggcgaccccatcctgaacagcacctactggtccggcagcctgatgatgacccggctggccgtgaagcctaagagcaatggcggcggatacaaccagcaccagctggccctgcggagcatcgagaagggcagatacgacaaagtgatgccctacggccccagcggcatcaagcagggcgacacactgtacttccccgccgtgggcttcctggtccggaccgagttcaagtacaacgacagcaactgccccatcaccaagtgccagtacagcaagcccgagaactgcagactgagcatgggcatccggcccaacagccactacatcctgcggagcggcctgctgaagtacaacctgagcgacggcgagaaccccaaagtcgtctttattgagatcagcgaccagcggctgtccatcggcagccccagcaagatctacgacagcctgggccagcccgtgttctaccaggccagcttcagctgggacaccatgatcaagttcggcgacgtgctgaccgtgaaccccctggtggtgaactggcggaacaataccgtgatcagcagacccggccagagccagtgcccccggttcaacacctgccccgcgatctgcgcggagggcgtgtacaacgacgccttcctgatcgaccggatcaactggatctctgccggcgtgttcctggactccaacgcgaccgccgcgaatcccgtgttcaccgtgtttaaggacaacgagatcctgtaccgggcccagctggccagcgaggacaccaacgcccagaaaaccatcaccaactgctttctgctgaagaacaagatctggtgcatcagcctggtggagatctacgataccggcgacaacgtgatcaggcccaagctgttcgccgtgaagatccccgagcagtgcaccggtggaggtggctctggtggaggaggctctggaggtggtggatcagcggcccagccggcccaggtgcagctggtgcagagcggagccgaggataagaaacctggcgctagcgtgaaggtgtcctgcaaggccagcggcttcaacatcaaggacacctacatccactgggtgcgccaggctccaggacagggcctggaatggatgggccggatcgaccccgccaacgacaacaccctgtacgccagcaagttccagggcagagtgaccatcaccgccgacaccagcagcaacaccgcctacatggaactgagcagcctgagaagcgaggacaccgccgtgtactactgtggcagaggctacggctactacgtgttcgaccactggggccagggcaccaccgtgacagtgtctagcggcggaggcggaagtggaggcggaggaagcggaggcggcggatccgacatcgtgatgacccagagccctagcagcctgtctgccagcgtgggcgaccgcgtgaccattacctgccggaccagccggtccatcagccagtacctggcctggtatcaggaaaagcccggcaaggcccccaagctgctgatctacagcggcagcaccctgcagagcggcgtgccaagcagattcagcggcagcggctccggcaccgacttcaccctgaccatcagcagcctgcagcccgaggacttcgccacctactactgccagcagcacaacgagaaccccctgaccttcgggcagggaacaaaggtggagatcaagagagcggccgcaagaggttctcatcaccatcaccatcac

SEQ ID NO: 58 - protein, 842 aa, Synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSAAQPAQVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSGGGGSGGGGSGGGGSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKRAAARGSHHHHHH

SEQ ID NO: 59 - DNA, 723 bp, Synthetic construct
  source 1..723: mol_type = other DNA
caggtgcagctggtgcagagcggagccgaggataagaaacctggcgctagcgtgaaggtgtcctgcaaggccagcggcttcaacatcaaggacacctacatccactgggtgcgccaggctccaggacagggcctggaatggatgggccggatcgaccccgccaacgacaacaccctgtacgccagcaagttccagggcagagtgaccatcaccgccgacaccagcagcaacaccgcctacatggaactgagcagcctgagaagcgaggacaccgccgtgtactactgtggcagaggctacggctactacgtgttcgaccactggggccagggcaccaccgtgacagtgtctagcggcggaggcggaagtggaggcggaggaagcggaggcggcggatccgacatcgtgatgacccagagccctagcagcctgtctgccagcgtgggcgaccgcgtgaccattacctgccggaccagccggtccatcagccagtacctggcctggtatcaggaaaagcccggcaaggcccccaagctgctgatctacagcggcagcaccctgcagagcggcgtgccaagcagattcagcggcagcggctccggcaccgacttcaccctgaccatcagcagcctgcagcccgaggacttcgccacctactactgccagcagcacaacgagaaccccctgaccttcgggcagggaacaaaggtggagatcaagaga

SEQ ID NO: 60 - protein, 241 aa, Synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSGGGGSGGGGSGGGGSDIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKR

SEQ ID NO: 61 - DNA, 1803 bp, Synthetic construct
  source 1..1803: mol_type = other DNA
atgaagaagatcaacgagggcctgctggacagcaagatcctgagcgccttcaacaccgtgatcgccctgctgggcagcatcgtgatcattgtgatgaacatcatgatcatccagaactacaccagaagcaccgacaaccaggccgtgatcaaggacgctctccaggggatccagcagcagatcaagggcctggccgacaagatcggcaccgagatcggccccaaggtgtccctgatcgacaccagcagcaccatcaccatccccgccaacatcggcctgctggggtccaagatcagccagagcaccgccagcatcaacgagaacgtgaacgagaagtgcaagttcaccctgccccccctgaagatccacgagtgcaacatcagctgccccaaccccctgcccttccgggagtaccggccccagaccgagggcgtgagcaacctggtcggcctgcccaacaacatctgcctgcagaaaaccagcaaccagatcctgaagcccaagctcatttcctacaccctgcccgtggtgggccagagcggcacctgcatcaccgaccccctgctggccatggacgagggctacttcgcctacagccacctggaacggatcggcagctgcagcaggggcgtgtccaagcagcggatcatcggcgtgggcgaggtgctggaccggggcgacgaggtgcccagcctgttcatgaccaacgtgtggaccccccccaaccccaacaccgtgtaccactgcagcgccgtgtacaacaacgagttctactacgtgctgtgcgccgtgagcaccgtgggcgaccccatcctgaacagcacctactggtccggcagcctgatgatgacccggctggccgtgaagcctaagagcaatggcggcggatacaaccagcaccagctggccctgcggagcatcgagaagggcagatacgacaaagtgatgccctacggccccagcggcatcaagcagggcgacacactgtacttccccgccgtgggcttcctggtccggaccgagttcaagtacaacgacagcaactgccccatcaccaagtgccagtacagcaagcccgagaactgcagactgagcatgggcatccggcccaacagccactacatcctgcggagcggcctgctgaagtacaacctgagcgacggcgagaaccccaaagtcgtctttattgagatcagcgaccagcggctgtccatcggcagccccagcaagatctacgacagcctgggccagcccgtgttctaccaggccagcttcagctgggacaccatgatcaagttcggcgacgtgctgaccgtgaaccccctggtggtgaactggcggaacaataccgtgatcagcagacccggccagagccagtgcccccggttcaacacctgccccgcgatctgcgcggagggcgtgtacaacgacgccttcctgatcgaccggatcaactggatctctgccggcgtgttcctggactccaacgcgaccgccgcgaatcccgtgttcaccgtgtttaaggacaacgagatcctgtaccgggcccagctggccagcgaggacaccaacgcccagaaaaccatcaccaactgctttctgctgaagaacaagatctggtgcatcagcctggtggagatctacgataccggcgacaacgtgatcaggcccaagctgttcgccgtgaagatccccgagcagtgcaccggtggaggtggctctggtggaggaggctctggaggtggtggatcagcggcccagccggccgcggccgcaagaggttctcatcaccatcaccatcac

SEQ ID NO: 62 - protein, 601 aa, Synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSAAQPAAAARGSHHHHHH

SEQ ID NO: 63 - protein, 602 aa, Synthetic construct
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQASKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVARPKLFAVKIPEQCT

SEQ ID NO: 64 - protein, 602 aa, Synthetic construct
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVARPKLFAVKIPEQCT

SEQ ID NO: 65 - protein, 569 aa, Synthetic construct
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQASKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLK

SEQ ID NO: 66 - protein, 569 aa, Synthetic construct
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLK

SEQ ID NO: 67 - protein, 568 aa, Synthetic construct
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQASKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLL

SEQ ID NO: 68 - protein, 568 aa, Synthetic construct
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLL

SEQ ID NO: 69 - protein, 524 aa, Synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 70 - protein, 1368 aa, Synthetic construct [sequence omitted]

(19) United States
(12) Patent Application Publication
(10) Pub. No.: US 2025/0205357 A1
(43) Pub. Date: Jun. 26, 2025

(54) TARGETED DELIVERY OF ARMMS

(71) Applicants: President and Fellows of Harvard College, Cambridge, MA (US); Vesigen, Inc., Cambridge, MA (US)

(72) Inventors: Quan Lu, Cambridge, MA (US); Zhi Qiao, Cambridge, MA (US); Chad Albert Cowan, Gloucester, MA (US)

(73) Assignees: President and Fellows of Harvard College, Cambridge, MA (US); Vesigen, Inc., Cambridge, MA (US)

(21) Appl. No.: 18/845,704

(22) PCT Filed: Mar. 13, 2023
(86) PCT No.: PCT/US2023/064262

§ 371 Date: Sep. 10, 2024

Related U.S. Application Data
(60) Provisional application No. 63/319,238, filed on Mar. 11, 2022.

(51) Int. Cl. A61K 47/69; A61K 35/17; A61K 47/68; A61K 48/00; C07K 14/005; C07K 14/47; C07K 16/28; C12N 9/22; C12N 15/11; C12N 15/88

(52) U.S. Cl. CPC A61K 47/6913; A61K 35/17; A61K 47/6849; A61K 48/0041; C07K 14/005; C07K 14/4702; C07K 16/2815; C12N 9/22; C12N 15/11; C12N 15/88; C07K 2317/622; C07K 2319/09; C12N 2310/20; C12N 2760/18222

(57) ABSTRACT

The present disclosure provides arrestin domain-containing protein 1 (ARRDC1)-mediated microvesicles (ARMMs) modified with Nipah virus (NiV) glycoproteins fused to antibodies or antigen-binding fragments and NiV fusion proteins, and variants thereof. The ARMMs of the present disclosure may be targeted to particular cell types and used to deliver agents (e.g., therapeutic agents) to target cells. Microvesicle-producing cells are also provided by the present disclosure. The present disclosure also provides methods of delivering molecules to target cells using the ARMMs described herein, and methods of treating a patient using any of the ARMMs or microvesicle-producing cells provided herein. Kits comprising any of the ARMMs or microvesicle-producing cells described herein are also provided by the present disclosure.

### RELATED APPLICATIONS

This application claims priority under 35 U.S.C. § 119(e) to U.S. Provisional Application, U.S. Ser. No. 63/319,238, filed Mar. 11, 2022, which is incorporated by reference herein.

### GOVERNMENT SUPPORT

This invention was made with government support under ES030990 awarded by the National Institutes of Health. The government has certain rights in the invention.

### REFERENCE TO AN ELECTRONIC SEQUENCE LISTING

The contents of the electronic sequence listing (H082470405WO00-SEQ-AZW.xml; Size: 115,494 bytes; and Date of Creation: Mar. 13, 2023) is herein incorporated by reference in its entirety.

### BACKGROUND OF THE INVENTION

Arrestin domain-containing protein 1 (ARRDC1)-mediated microvesicles (ARMMs) have been demonstrated to be able to package and deliver a myriad of therapeutic cargos such as proteins, RNAs, and genome-editors. However, there remains a need to deliver ARMMs and their cargo in a tissue or cell-specific manner.

### SUMMARY OF THE INVENTION

This invention provides microvesicles with increased cell specificity. The present disclosure provides evidence that surface modification (i.e., pseudotyping) of ARMMs with the Nipah virus (NiV) envelope proteins (glycoprotein [G] and fusion protein [F]) allows targeted delivery into specific cells of cargos contained in the vesicles. For example, it is shown that ARMMs decorated with engineered NiV-G protein that contains a CD8-targeting single-chain variable fragment (scFv) can deliver proteins, as well as mRNAs, into CD8⁺-T cells. Because the NiV-G protein can be engineered to contain other targeting moieties, ARMMs may thus be pseudotyed with such NiV-G proteins to allow for specific targeting of many other cell or tissue types. In addition, the present disclosure provides for envelope proteins with similar recognition and fusion activities from other viruses, such as the Measles virus, that can be used to modify ARMMs for targeted delivery.

Thus, in one aspect, the present disclosure provides ARRDC1-mediated microvesicles (ARMMs), comprising: (i) a lipid bilayer and an ARRDC1 protein or a variant thereof, (ii) a Nipah virus (NiV) glycoprotein variant fused to an antibody or antigen-binding fragment, wherein the NiV glycoprotein variant does not recognize and/or bind to an ephrin receptor, and (iii) a NiV fusion protein. In certain embodiments, the ARRDC1 protein comprises an amino acid sequence at least 80%, at least 85%, at least 90%, at least 95%, at least 96%, at least 97%, at least 98%, at least 99%, or 100% identical to the amino acid sequence of SEQ ID NO: 4. In certain embodiments, the NiV glycoprotein variant fused to an antibody or antigen binding fragment comprises an amino acid sequence at least 80%, at least 85%, at least 90%, at least 95%, at least 96%, at least 97%, at least 98%, at least 99%, or 100% identical to the amino acid sequence of SEQ ID NO: 58. In certain embodiments, the NiV fusion protein comprises an amino acid sequence at least 80%, at least 85%, at least 90%, at least 95%, at least 96%, at least 97%, at least 98%, at least 99%, or 100% identical to the amino acid sequence SEQ ID NOs: 69.

In some embodiments, the antibody or antigen binding fragment comprises a CD8 targeting antibody or antigen binding fragment. In certain embodiments, the antigen-binding fragment comprises an amino acid sequence at least 80%, at least 85%, at least 90%, at least 95%, at least 96%, at least 97%, at least 98%, at least 99%, or 100% identical to the amino acid sequence of SEQ ID NO: 60.

In another aspect, the present disclosure provides microvesicle-producing cells comprising: a first isolated nucleic acid encoding an ARRDC1 protein or a variant thereof, a second isolated nucleic acid encoding a Nipah virus (NiV) glycoprotein variant fused to an antibody or antigen-binding fragment, wherein the NiV glycoprotein variant does not recognize and/or bind to an ephrin receptor, and a third isolated nucleic acid encoding a NiV fusion protein. In some embodiments, the first isolated nucleic acid, the second isolated nucleic acid, and the third isolated nucleic acid are expressed on the same recombinant expression construct under the control of one or more heterologous promoters. In some embodiments, the first isolated nucleic acid, the second isolated nucleic acid, and the third isolated nucleic acid are expressed on more than one recombinant expression construct, wherein the one or more recombinant expression constructs are under the control of one or more heterologous promoters. In certain embodiments, the first isolated nucleic acid, the second isolated nucleic acid, and the third isolated nucleic acid are expressed on different recombinant expression constructs, wherein the different recombinant expression constructs are each under the control of a heterologous promoter.

In some embodiments, the antibody or antigen-binding fragment used in the microvesicles described herein comprises a single-chain variable fragment (scFv). In certain embodiments, the antibody or antigen-binding fragment comprises a nanobody. In certain embodiments, the antibody or antigen-binding fragment comprises an antibody mimetic protein, such as a designed ankyrin repeat protein (DARPin). The antibody or antigen-binding fragment of the microvesicles described herein may be modified to target specific cell types, or a single cell type. In some embodiments, the antibody or antigen-binding fragment targets a particular cell type (e.g., neurons, astrocytes, oligodendrocytes, microglial cells, T-cells, dendritic cells, B cells, NK cells, stem cells, progenitor cells, endothelial cells, muscle cells, myocardial cells, epithelial cells, or hepatic cells). In certain embodiments, the antibody or antigen-binding fragment targets T cells. In certain embodiments, the antibody or antigen-binding fragment targets neurons. In certain embodiments, the antibody or antigen-binding fragment binds selectively to an antigen expressed by target cell. In certain embodiments, the target cell is a CD8 expressing cell. In certain embodiments, the target cell is a T cell.

In various embodiments, the NiV glycoprotein variant and NiV fusion protein used in the microvesicles described herein may be modified (e.g., with amino acid mutations or truncations) relative to the wild-type NiV glycoprotein and fusion protein. In some embodiments, the NiV glycoprotein variant comprises one or more amino acid substitutions relative to a wild-type NiV glycoprotein of SEQ ID NO: 1. The sequence of wild-type NiV glycoprotein can also be found at https://www.uniprot.org/uniprot/Q9IH62. In some embodiments, the one or more amino acid substitutions are selected from the group consisting of Y389X, E501X, W504X, Q530X, E533X, and I588X, wherein X is any amino acid. In some embodiments, the one or more amino acid substitutions are selected from the group consisting of Y389A, E501A, W504A, Q530A, E533A, and I588A. In certain embodiments, the NiV glycoprotein variant comprises amino acid substitutions E501A, W504A, Q530A, and E533A relative to a wild-type NiV glycoprotein of SEQ ID NO: 1.

In addition to the amino acid substitutions described herein, both the NiV glycoprotein and NiV fusion protein may also be truncated for use in the presently described microvesicles. In some embodiments, the NiV glycoprotein variant further comprises a C-terminal truncation. In certain embodiments, the NiV glycoprotein variant further comprises a C-terminal truncation of about 33 or about 34 amino acids in length. In some embodiments, the NiV fusion protein comprises a C-terminal truncation. In certain embodiments, the NiV fusion protein comprises a C-terminal truncation of about 22 amino acids in length.

In various embodiments, the NiV glycoprotein variant comprises an amino acid sequence at least 80%, at least 85%, at least 90%, at least 95%, at least 96%, at least 97%, at least 98%, or at least 99% identical to the amino acid sequence of any one of SEQ ID NOs: 63-68. In certain embodiments, the NiV glycoprotein variant comprises the amino acid sequence of any one of SEQ ID NOs: 63-68. In certain embodiments, the NiV fusion protein comprises an amino acid sequence at least 80%, at least 85%, at least 90%, at least 95%, at least 96%, at least 97%, at least 98%, or at least 99% identical to the amino acid sequence of SEQ ID NO: 6. In certain embodiments, the NiV fusion protein comprises the amino acid sequence of SEQ ID NO: 6. In certain embodiments, the ARRDC1 protein comprises an amino acid sequence at least 80%, at least 85%, at least 90%, at least 95%, at least 96%, at least 97%, at least 98%, or at least 99% identical to the amino acid sequence of any one of SEQ ID NOs: 3-5. In certain embodiments, the ARRDC1 protein comprises the amino acid sequence of any one of SEQ ID NOs: 3-5.

In various embodiments, the microvesicles described herein may further comprise an agent. In certain embodiments, the agent is fused to the ARRDC1 protein. In certain embodiments, the ARRDC1 protein is fused to TAR. In certain embodiments, TAR recruits an agent fused to Tat, optionally wherein the agent is an RNA. In certain embodiments, the agent is fused to a WW domain, optionally wherein the agent is a protein. In certain embodiments, the WW domain comprises an amino acid sequence of any one of SEQ ID NOs: 7-44, or an amino acid sequence at least 80%, at least 85%, at least 90%, at least 95%, at least 96%, at least 97%, at least 98%, or at least 99% identical to the amino acid sequence of any one of SEQ ID NOs: 7-44.

In some embodiments, the agent is selected from the group consisting of a nucleic acid, a protein, and a small molecule. In some embodiments, the nucleic acid comprises an RNA. In certain embodiments, the nucleic acid comprises an RNAi agent (e.g., a coding RNA, a non-coding RNA, an antisense RNA, an mRNA, a small RNA, an siRNA, an shRNA, a microRNA, an snRNA, a snoRNA, a lincRNA, a structural RNA, a ribozyme, or a precursor thereof). In some embodiments, the nucleic acid comprises a DNA. In certain embodiments, the DNA comprises a retrotransposon sequence, a LINE sequence, a SINE sequence, a composite SINE sequence, or an LTR-retrotransposon sequence. In some embodiments, the nucleic acid encodes a protein.

In some embodiments, the agent comprises a detectable label. In some embodiments, the agent comprises a therapeutic agent. In certain embodiments, the agent is selected from the group consisting of an enzyme, an antibody, a Fab, a Fab′, a F(ab′)2, a Fd, a scFv, a Fv, a dsFv, a diabody, and an affibody. The agent may also comprise a protein. For example, in some embodiments, the agent comprises a transcription factor, a transcriptional repressor, a fluorescent protein, a kinase, a phosphatase, a protease, a ligase, or a recombinase. In certain embodiments, the agent comprises a cytotoxic agent. In some embodiments, the agent is covalently bound to the ARRDC1 protein or a variant thereof. For example, the agent may be conjugated to the ARRDC1 protein or a variant thereof via a linker (e.g., a cleavable linker). In some embodiments, the linker comprises a protease recognition site or a UV-cleavable moiety. In some embodiments, the agent is a fusion protein. In some embodiments, the agent comprises a nuclease. In some embodiments, the agent comprises a zinc finger nuclease (ZFN). In some embodiments, the agent comprises a TALEN. In some embodiments, the agent comprises a Cas protein (e.g., a Cas9 protein), or a variant thereof. In certain embodiments, the Cas9 protein is a Cas9 nickase (nCas9) or a nuclease-inactivated Cas9 (dCas9). In certain embodiments, the Cas9 protein or variant thereof is fused to at least one nuclear localization sequence (NLS). In certain embodiments, the microvesicle further comprises a guide RNA (gRNA).

In some embodiments, the fusion protein comprises a nuclease. In certain embodiments, the fusion protein comprises a ZFN. In certain embodiments, the fusion protein comprises a TALEN. In certain embodiments, the fusion protein comprises a Cas protein. In certain embodiments, In certain embodiments, In certain embodiments, In certain embodiments, the microvesicle further comprising a guide RNA (gRNA).

In some embodiments, the agent comprises a detectable label. In certain embodiments, the agent comprises a therapeutic agent. In certain embodiments, the agent comprises a cytotoxic agent.

In some embodiments, the present disclosure provides one or more isolated nucleic acids encoding the ARRDC1 protein, the NiV glycoprotein variant, and the NiV fusion protein of the microvesicle of the disclosure. In some embodiments, the present disclosure provides one or more vectors comprising the one or more isolated nucleic acids of the disclosure. In certain embodiments, the present disclosure provides a vector comprising the isolated nucleic acid of the disclosure.

In some embodiments, the present disclosure provides microvesicle-producing cell comprising a first isolated nucleic acid encoding an ARRDC1 protein or a variant thereof, a second isolated nucleic acid encoding a Nipah virus (NiV) glycoprotein variant fused to an antibody or antigen-binding fragment, wherein the NiV glycoprotein variant does not recognize and/or bind to an ephrin receptor, and a third isolated nucleic acid encoding a NiV fusion protein. In certain embodiments, the ARRDC1 protein comprises an amino acid sequence at least 80%, at least 85%, at least 90%, at least 95%, at least 96%, at least 97%, at least 98%, at least 99%, or 100% identical to the amino acid sequence of SEQ ID NO: 4. In certain embodiments, the NiV glycoprotein variant fused to an antibody or antigen binding fragment comprises an amino acid sequence at least 80%, at least 85%, at least 90%, at least 95%, at least 96%, at least 97%, at least 98%, at least 99%, or 100% identical to the amino acid sequence of SEQ ID NO: 58. In certain embodiments, the NiV fusion protein comprises an amino acid sequence at least 80%, at least 85%, at least 90%, at least 95%, at least 96%, at least 97%, at least 98%, at least 99%, or 100% identical to the amino acid sequence of SEQ ID NO: 69. In certain embodiments, the antigen-binding fragment comprises an amino acid sequence at least 80%, at least 85%, at least 90%, at least 95%, at least 96%, at least 97%, at least 98%, at least 99%, or 100% identical to the amino acid sequence of SEQ ID NO: 60.

In some embodiments, the first isolated nucleic acid, the second isolated nucleic acid, and the third isolated nucleic acid are expressed on the same recombinant expression construct under the control of one or more heterologous promoters. In certain embodiments, the first isolated nucleic acid, the second isolated nucleic acid, and the third isolated nucleic acid are expressed on more than one recombinant expression construct, wherein the more than one recombinant expression constructs are under the control of one or more heterologous promoters. the first isolated nucleic acid, the second isolated nucleic acid, and the third isolated nucleic acid are each expressed on different recombinant expression constructs, wherein the different recombinant expression constructs are each under the control of a heterologous promoter.

In some embodiments, the antibody or antigen-binding fragment used in the microvesicles described herein comprises a single-chain variable fragment (scFv). In certain embodiments, the antibody or antigen-binding fragment comprises a nanobody. In certain embodiments, the antibody or antigen-binding fragment comprises an antibody mimetic protein, such as a designed ankyrin repeat protein (DARPin). The antibody or antigen-binding fragment of the microvesicles described herein may be modified to target specific cell types, or a single cell type. In some embodiments, the antibody or antigen-binding fragment targets a particular cell type (e.g., neurons, astrocytes, oligodendrocytes, microglial cells, T-cells, dendritic cells, B cells, NK cells, stem cells, progenitor cells, endothelial cells, muscle cells, myocardial cells, epithelial cells, or hepatic cells). In certain embodiments, the antibody or antigen-binding fragment targets T cells. In certain embodiments, the antibody or antigen-binding fragment targets neurons. In certain embodiments, the antibody or antigen-binding fragment binds selectively to an antigen expressed by target cell. In certain embodiments, the target cell is a CD8 expressing cell. In certain embodiments, the target cell is a T cell.

In various embodiments, the NiV glycoprotein variant and NiV fusion protein used in the microvesicles described herein may be modified (e.g., with amino acid mutations or truncations) relative to the wild-type NiV glycoprotein and fusion protein. In some embodiments, the NiV glycoprotein variant comprises one or more amino acid substitutions relative to a wild-type NiV glycoprotein of SEQ ID NO: 1. The sequence of wild-type NiV glycoprotein can also be found at https://www.uniprot.org/uniprot/Q9IH62. In some embodiments, the one or more amino acid substitutions are selected from the group consisting of Y389X, E501X, W504X, Q530X, E533X, and I588X, wherein X is any amino acid. In some embodiments, the one or more amino acid substitutions are selected from the group consisting of Y389A, E501A, W504A, Q530A, E533A, and I588A. In certain embodiments, the NiV glycoprotein variant comprises amino acid substitutions E501A, W504A, Q530A, and E533A relative to a wild-type NiV glycoprotein of SEQ ID NO: 1.

In addition to the amino acid substitutions described herein, both the NiV glycoprotein and NiV fusion protein may also be truncated for use in the presently described microvesicles. In some embodiments, the NiV glycoprotein variant further comprises a C-terminal truncation. In certain embodiments, the NiV glycoprotein variant further comprises a C-terminal truncation of about 33 or about 34 amino acids in length. In some embodiments, the NiV fusion protein comprises a C-terminal truncation. In certain embodiments, the NiV fusion protein comprises a C-terminal truncation of about 22 amino acids in length.

In various embodiments, the NiV glycoprotein variant comprises an amino acid sequence at least 80%, at least 85%, at least 90%, at least 95%, at least 96%, at least 97%, at least 98%, or at least 99% identical to the amino acid sequence of any one of SEQ ID NOS: 63-68. In certain embodiments, the NiV glycoprotein variant comprises the amino acid sequence of any one of SEQ ID NOs: 63-68. In certain embodiments, the NiV fusion protein comprises an amino acid sequence at least 80%, at least 85%, at least 90%, at least 95%, at least 96%, at least 97%, at least 98%, or at least 99% identical to the amino acid sequence of SEQ ID NO: 6. In certain embodiments, the NiV fusion protein comprises the amino acid sequence of SEQ ID NO: 6. In certain embodiments, the ARRDC1 protein comprises an amino acid sequence at least 80%, at least 85%, at least 90%, at least 95%, at least 96%, at least 97%, at least 98%, or at least 99% identical to the amino acid sequence of any one of SEQ ID NOs: 3-5. In certain embodiments, the ARRDC1 protein comprises the amino acid sequence of any one of SEQ ID NOs: 3-5.

In another aspect, the present disclosure provides methods of delivering a molecule to a target cell comprising contacting the target cell with any of the microvesicles described herein. In some embodiments, the target cells are T cells. In some embodiments, the methods described herein are performed in vitro. In some embodiments, the methods described herein are performed ex vivo. In some embodiments, the methods described herein are performed in vivo.

In another aspect, the present disclosure provides methods of treating a patient consisting of administering to the patient any of the microvesicles described herein.

In another aspect, the present disclosure provides methods of treating a patient consisting of administering to the patient any of the microvesicle-producing cells described herein.

In another aspect, the present disclosure provides methods of delivering ARMMs, comprising delivering any of the ARMMs described herein, or any of the microvesicle-producing cells described herein, to a subject. In some embodiments, the subject is mammalian. In certain embodiments, the subject is human.

In another aspect, the present disclosure provides kits comprising one or more of the ARMMs described herein, or one or more of the microvesicle-producing cells described herein.

Other advantages, features, and uses of the invention will be apparent from the detailed description of certain exemplary, non-limiting embodiments, the drawings, the non-limiting working examples, and the claims.

### BRIEF DESCRIPTION OF THE DRAWINGS

FIG. 1 provides a non-limiting schematic of a NiV-ARMM. The envelope of Nipah virus (NiV) contains two proteins: the glycoprotein (G) and the fusion protein (F). The G protein has a cytoplasmic tail, a transmembrane domain (TM), an ectodomain (ED), and a targeting domain that recognizes receptors on recipient cells. The endogenous targeting domain of NiV can be replaced with targeting moieties, such as the single chain antibody variant fragments (scFv), allowing specific targeting of particular cells or tissue. The F protein has a cytoplasmic tail, TM, and ED. When co-expressed with ARRDC1 and its associated cargos, NiV F/G protein may be incorporated onto ARMMs, thus allowing targeting of the vesicles to specific cells or tissue.

FIG. 2 shows the incorporation of NiV F and G proteins into ARMMs. HEK293T cells were co-transfected with NiV-G (or NiV-G-CD8, which contains CD8-targeting scFv) and NiV-F together with or without ARRDC1-GFP. NiV-G proteins are His-tagged at the C-terminus; NiV F is tagged with AU-1. About 48 hours post transfection, extracellular vesicles (EVs) were pelleted by ultracentrifugation. Both cell and EV lysates were subjected to anti-GFP, anti-AU1, anti-His, anti-CD9, and anti-Vinculin Western blotting.

FIG. 3 shows the co-segregation of NiV-G with ARRDC1 in extracellular vesicles. HEK293T cells were co-transfected with NiV-G (His-tagged) and NiV-F together with ARRDC1-GFP. About 48 hours post transfection, extracellular vesicles (EVs) were pelleted by ultracentrifugation and then subjected to sucrose gradient ultracentrifugation. Ten fractions were prepared, and each was spun down by ultracentrifugation. Pellets from each of the ten fractions were used for anti-His, anti-GFP, and anti-CD9 western blotting.

FIG. 4 shows specific targeting of NiV-G-CD8-decorated ARMMs to CD8⁺ T cells. HEK293T cells were transfected with ARRDC1-GFP along with 1) control vector, 2) VSVG, 3) NiV-F/G that lacks any targeting capability (NiV-NT), or 4) NiV-F/G that contains CD8-scFV as the targeting domain, to produce unmodified ARMMs containing GFP (ARMM (GFP)), VSVG-pseudotyped ARMMs (VSVG-ARMM (GFP)), nontargeting NiV-decorated ARMMs (NiV-NT-ARMM (GFP)), or NiV-CD8-ARMM (GFP), respectively. All ARMMs contain GFP as cargo. Freshly isolated human PBMCs (peripheral blood mononuclear cells) were incubated with control PBS (untreated), ARMM (GFP), VSVG-ARMM (GFP), NiV-NT-ARMM (GFP), or NiV-CD8-ARMM (GFP) at an ARMMs/cell ratio of 2×10⁵ for 90 min, stained with APC-coupled anti-CD8 antibody, and then subjected to flow cytometry analysis (with Y-axis for CD8 and X-axis for GFP signal).

FIG. 5 provides flow cytometry data showing that selective targeting in PBMCs by NiV-CD8-ARMM is dose-dependent. Freshly isolated human PBMC were incubated with NiV-CD8-ARMM (GFP) at an ARMMs/cell ratio of 1×10⁴, 5×10⁴, 1×10⁵, 2×10⁵, or 4×10⁵, stained with APC-coupled anti-CD8 antibody, and then subjected to flow cytometry analysis (with Y-axis for CD8 and X-axis for GFP signal). Representative flow cytometry plots out of five independent experiments are shown.

FIG. 6 shows Vectofusin-1 increases NiV-ARMM uptake by PBMCs in a non-specific manner. Freshly isolated human PBMCs were incubated with NiV-NT-ARMM (GFP) or NiV-CD8-ARMM (GFP) at an ARMMs/cell ratio of 2×10⁵ in the presence or absence of two different concentrations (2 or 10 μg/ml) of Vectofusin-1. Cells were then subjected to anti-CD8 staining and flow cytometry. Representative flow cytometry plots out of six independent experiments are shown.

FIG. 7 shows selective targeting of CD8⁺ T cells in activated PBMC by NiV-CD8-ARMMs. Freshly isolated human PBMC were activated for three days (using IL2, CD3, and CD28 antibodies) and then incubated with NiV-NT-ARMM (GFP) or NiV-CD8-ARMM (GFP) at an ARMMs/cell ratio of 2×10⁵ in the presence or absence of Vectofusin-1, respectively. Cells were then subjected to anti-CD8 staining and flow cytometry. Representative flow cytometry plots out of four independent experiments are shown.

FIG. 8 shows selective functional delivery of mRNA into CD8⁺ T cells via NiV-CD8-ARMM. HEK293T cells were co-transfected with ARRDC1-Tat, TAR-GFP-mRNA along with NiV-F and NiV-G-CD8 (or NiV-G-NT), to produce NiV-decorated ARMMs that contain GFP-mRNA as the cargo. About 48 hours post transfection, ARMMs were pelleted by ultracentrifugation and resuspended in PBS. Freshly isolated human PBMCs were incubated with NiV-NT-ARMM (GFP-mRNA) or NiV-CD8-ARMM (GFP-mRNA) at an ARMMs/cell ratio of 2×10⁵. Cells were then subjected to anti-CD8 staining and flow cytometry. Representative flow cytometry plots out of two independent experiments are shown.

FIG. 9 shows that no carry over of GFP protein into ARMMs that contain GFP mRNA was observed. Western blotting showed GFP protein not budding into NiV-CD8-ARMM (GFP-mRNA). HEK293T cells were transfected with ARRDC1-GFP, or ARRDC1-TAT plus TAR-GFP together with NiV-F and G (or G-CD8). Extracellular vesicles (EVs) were pelleted by ultracentrifugation. Both cell and EV lysates were subjected to anti-GFP, anti-CD9, and anti-Vinculin Western blotting.

### DEFINITIONS

As used herein, the term “agent” refers to a substance that can be incorporated in an ARMM, for example, into the liquid phase of the ARMM or into the lipid bilayer of the ARMM. In some embodiments, the agent is an agent to be delivered to a target cell. In some embodiments, the agent is a biologically active agent, i.e., it has activity in a cell, biological system, and/or subject. For instance, a substance that, when administered to a subject, has a biological effect on that subject, is considered to be biologically active. In some embodiments, an agent is a therapeutic agent. As used herein, the term “therapeutic agent” refers to any agent that, when administered to a subject, has a therapeutic, diagnostic, and/or prophylactic effect and/or elicits a desired biological and/or pharmacological effect. As used herein, the term “therapeutic agent” may be a nucleic acid that is delivered to a cell via its association with or inclusion into an ARMM. In certain embodiments, the agent is a nucleic acid. In some embodiments, the nucleic acid encodes a protein. In certain embodiments, the agent is DNA (e.g., a retrotransposon sequence, a LINE sequence, a SINE sequence, a composite SINE sequence, or an LTR-retrotransposon sequence). In certain embodiments, the agent is RNA (e.g., an RNAi agent, a coding RNA, a non-coding RNA, an antisense RNA, an mRNA, a small RNA, an siRNA, an shRNA, a microRNA, an snRNA, a snoRNA, a lincRNA, a structural RNA, a ribozyme, or a precursor thereof). the agent is selected from the group consisting of an enzyme, an antibody, a Fab, a Fab′, a F(ab′)2, a Fd, a scFv, a Fv, a dsFv, a diabody, and an affibody. In certain embodiments, the agent is a peptide or protein. In some embodiments, the protein is a transcription factor, a transcriptional repressor, a fluorescent protein, a kinase, a phosphatase, a protease, a ligase, or a recombinase. In certain embodiments, the agent to be delivered is a small molecule. In some embodiments, the small molecule is an FDA-approved drug. In some embodiments, the agent is a fusion protein. In certain embodiments, the agent comprises a nuclease. In certain embodiments, the agent comprises a zinc finger nuclease. In certain embodiments, the agent comprises a TALEN. In certain embodiments, the agent comprises a Cas protein. In certain embodiments, the agent comprises a Cas9 protein, or a variant thereof (e.g., nCas9 or dCas9). In some embodiments, the agent to be delivered is a diagnostic agent. In some embodiments, the agent to be delivered is useful as an imaging agent. In some embodiments, the agent comprises a detectable label.

An “antibody” refers to a glycoprotein belonging to the immunoglobulin superfamily. With some exceptions, mammalian antibodies are typically made of basic structural units each with two large heavy chains and two small light chains. There are several different types of antibody heavy chains, and several different kinds of antibodies, which are grouped together into different isotypes based on which heavy chain they possess. Five different antibody isotypes are known in mammals (IgG, IgA, IgE, IgD, and IgM), which perform different roles, and help direct the appropriate immune response for each different type of foreign object they encounter. Antibodies (and antigen-binding fragments) may be used in the present disclosure to target ARMMs to a particular cell type. The term “antibody” as used herein also encompasses antibody fragments and nanobodies, as well as variants of antibodies and variants of antibody fragments and nanobodies. In some embodiments, an antibody is a nanobody. In some embodiments, an antibody is a single chain variable fragment (scFv).

As used herein, the terms “approximately” and “about,” as applied to one or more values of interest, refer to a value that is similar to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (for example, when such number would exceed 100% of a possible value).

As used herein, the terms “Arrestin domain-containing protein 1” and “ARRDC1” or variants thereof refer to a protein that comprises a PSAP (SEQ ID NO: 2) and a PPXY motif, also referred to herein as a PSAP (SEQ ID NO: 2) and PPXY motif, respectively, in its C-terminus, and interacts with TSG101. Exemplary, non-limiting ARRDC1 protein sequences are provided herein, and additional, suitable ARRDC1 protein variants according to aspects of this invention are known in the art. For example, ARRDC1 variants are described in detail in PCT Publication WO2021/062196; the entire contents of which are hereby incorporated by reference in its entirety. It will be appreciated by those of skill in the art that this invention is not limited in this respect. Exemplary ARRDC1 sequences include the following (PSAP (SEQ ID NO: 2) and PPXY motifs are marked):

>gi|22748653|ref|NP_689498.1| arrestin domain-containing protein 1
[Homo sapiens]
(SEQ ID NO: 3)
MGRVQLFEISLSHGRVVYSPGEPLAGTVRVRLGAPLPFRAIRVTCIGSCGVSNKANDT
AWVVEEGYFNSSLSLADKGSLPAGEHSFPFQFLLPATAPTSFEGPFGKIVHQVRAAIH
TPRFSKDHKCSLVFYILSPLNLNSIPDIEQPNVASATKKFSYKLVKTGSVVLTASTDLR
GYVVGQALQLHADVENQSGKDTSPVVASLLQKVSYKAKRWIHDVRTIAEVEGAGV
KAWRRAQWHEQILVPALPQSALPGCSLIHIDYYLQVSLKAPEATVTLPVFIGNIAVNH

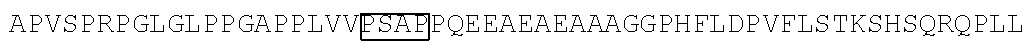

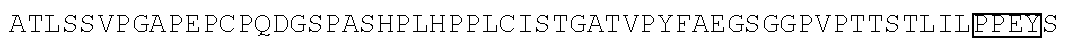

SWGYPYEAPPSYEQSCGGVEPSLTPES
>gi|244798004|ref|NP_001155957.1| arrestin domain-containing protein 1 isoform a
[Mus musculus]
(SEQ ID NO: 4)
MGRVOLFEIRLSQGRVVYGPGEPLAGTVHLRLGAPLPFRAIRVTCMGSCGVSTKAND
GAWVVEESYFNSSLSLADKGSLPAGEHNFPFQFLLPATAPTSFEGPFGKIVHQVRASI
DTPRFSKDHKCSLVFYILSPLNLNSIPDIEQPNVASTTKKFSYKLVKTGNVVLTASTDL
RGYVVGQVLRLQADIENQSGKDTSPVVASLLQKVSYKAKRWIYDVRTIAEVEGTGV
KAWRRAQWQEQILVPALPQSALPGCSLIHIDYYLQVSMKAPEATVTLPLFVGNIAVN

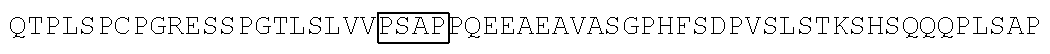

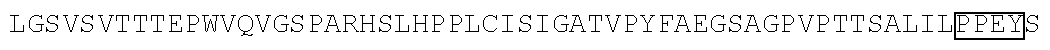

SWGYPYEAPPSYEQSCGAAGTDLGLIPGS
>gi|244798112|ref|NP_848495.2| arrestin domain-containing protein 1 isoform b
[Mus musculus]
(SEQ ID NO: 5)
MGRVOLFEIRLSQGRVVYGPGEPLAGTVHLRLGAPLPFRAIRVTCMGSCGVSTKAND
GAWVVEESYFNSSLSLADKGSLPAGEHNFPFQFLLPATAPTSFEGPFGKIVHQVRASI
DTPRFSKDHKCSLVFYILSPLNLNSIPDIEQPNVASTTKKFSYKLVKTGNVVLTASTDL
RGYVVGQVLRLQADIENQSGKDTSPVVASLLQVSYKAKRWIYDVRTIAEVEGTGVK
AWRRAQWQEQILVPALPQSALPGCSLIHIDYYLQVSMKAPEATVTLPLFVGNIAVNQ

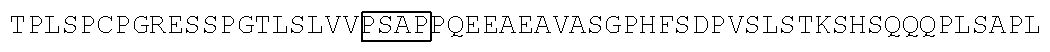

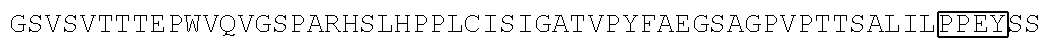

WGYPYEAPPSYEQSCGAAGTDLGLIPGS.

The terms “ARRDC1-mediated microvesicle” or “ARMM,” as used herein, refer to a microvesicle comprising an ARRDC1 protein or variant thereof, and/or TSG101 protein, or variant thereof. ARMMs have been described in detail, for example, in PCT application number PCT/US2013/024839, filed Feb. 6, 2013 (published as WO 2013/119602 A1 on Aug. 15, 2013) by Lu et al., and entitled “Arrdc1-Mediated Microvesicles (ARMMs) and Uses Thereof,” as well as in U.S. Pat. Nos. 9,737,480; 9,816,080; 10,260,055, 10,945,954, 11,001,817, and PCT Publications WO2018/067546, WO2021/062196, and WO2021/252924; the entire contents of which are hereby incorporated by reference in their entirety. In some embodiments, the ARMM is shed from a cell, and comprises an agent, for example, a nucleic acid, protein, or small molecule, present in the cytoplasm or associated with the membrane of the cell. In some embodiments, the ARMM is shed from a transgenic cell comprising a recombinant expression construct that includes a transgene, and the ARMM comprises a gene product, for example, an RNA transcript and/or a protein (e.g., an ARRDC1-Tat fusion protein and a TAR-payload RNA) encoded by the expression construct. In some embodiments, the ARMM is produced synthetically, for example, by contacting a lipid bilayer with an ARRDC1 protein or a variant thereof, or a variant thereof, in a cell-free system in the presence of TSG101, or a variant thereof. In other embodiments, the ARMM is synthetically produced by contacting a lipid bilayer with HECT domain ligase, and VPS4a. In some embodiments, an ARMM lacks a late endosomal marker. Some of the ARMMs provided herein do not include, or are negative for, one or more exosomal biomarkers. Exosomal biomarkers are known to those of skill in the art and include, but are not limited to, CD63, Lamp-1, Lamp-2, CD9, HSPA8, GAPDH, CD81, SDCBP, PDCD6IP, ENO1, ANXA2, ACTB, YWHAZ, HSP90AA1, ANXA5, EEF1A1, YWHAE, PPIA, MSN, CFL1, ALDOA, PGK1, EEF2, ANXA1, PKM2, HLA-DRA, and YWHAB. Certain ARMMs provided herein may include an exosomal biomarker. Accordingly, some ARMMs may be negative for one or more other exosomal biomarkers, but positive for one or more different exosomal biomarkers. For example, such an ARMM may be negative for CD63 and Lamp-1 but may include PGK1 or GAPDH; or may be negative for CD63, Lamp-1, CD9, and CD81, but may be positive for HLA-DRA. In some embodiments, ARMMs include an exosomal biomarker, but at a lower level than the level found in exosomes. For example, some ARMMs include one or more exosomal biomarkers at a level of less than about 1%, less than about 5%, less than about 10%, less than about 20%, less than about 30%, less than about 40%, or less than about 50% of the level of that biomarker found in exosomes. To give a non-limiting example, in some embodiments, an ARMM may be negative for CD63 and Lamp-1, include CD9 at a level of less than about 5% of the level of CD9 typically found in exosomes, and be positive for ACTB. Exosomal biomarkers in addition to those listed above are known to those of skill in the art, and the invention is not limited in this regard. In some embodiments, ARMMs provided by the present disclosure may comprise a NiV glycoprotein (or a variant thereof) and a NiV fusion protein (or a variant thereof).

As used herein, the terms “associated with,” “conjugated,” “linked,” “attached,” and “tethered,” when used with respect to two or more entities, for example, moieties, molecules, and/or ARMMs, means that the entities are physically associated or connected with one another, either directly or via one or more additional moieties that serve as a linker, to form a structure that is sufficiently stable so that the entities remain physically associated under the conditions in which the structure is used, e.g., physiological conditions. An ARMM is typically associated with an agent, for example, a nucleic acid, protein, or small molecule, by a mechanism that involves a covalent or non-covalent association. In certain embodiments, the agent is covalently bound to a molecule that is part of the ARMM, for example, an ARRCD1 protein or fragment thereof, a TSG101 protein or fragment thereof, or a lipid or protein that forms part of the lipid bilayer of the ARMM. In some embodiments, a peptide or protein is associated with an ARRCD1 protein or fragment thereof, a TSG101 protein or fragment thereof, or a lipid bilayer-associated protein by a covalent bond (e.g., an amide bond). In some embodiments, the association is via a linker, for example, a cleavable linker. In some embodiments, an entity is associated with an ARMM by inclusion in the ARMM, for example, by encapsulation of an entity (e.g., an agent) within the ARMM. For example, in some embodiments, an agent present in the cytoplasm of an ARMM-producing cell is associated with an ARMM by encapsulation of agent-comprising cytoplasm in the ARMM upon ARMM budding. Similarly, a membrane protein, or other molecule associated with the cell membrane of an ARMM producing cell, may be associated with an ARMM produced by the cell by inclusion into the ARMM membrane upon budding.

The term “Cas9” or “Cas9 protein” refers to an RNA-guided nuclease comprising a Cas9 protein, or a variant thereof (e.g., a protein comprising an active, inactive, or altered DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences are disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain. In some embodiments, a Cas9 protein, or a variant thereof, is delivered to a target cell using any of the microvesicles disclosed herein. In certain embodiments, the microvesicle further comprises a gRNA.

In some embodiments, a Cas9 protein comprises Streptococcus pyogenes Cas9 of SEQ ID NO: 70, or a variant having at least 80%, 85%, 90%, 95%, 96%, 97%, 98%, or 99% identity with SEQ ID NO: 70:

(SEQ ID NO: 70)
MDKKYSIGLDIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRVNTEITKA
PLSASMIKRYDEHHQDLTLLKALVRQQLPEKYKEIFFDQSKNGYA
GYIDGGASQEEFYKFIKPILEKMDGTEELLVKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFLKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGITIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD

In some embodiments, a Cas9 protein is a Cas9 nickase in which the nuclease activity of either the RuvC or the HNH domain has been inactivated. In some embodiments, a Cas9 nickase comprises a D10A or an H840A mutation relative to a wild type Streptococcus pyogenes Cas9 of SEQ ID NO: 70, or a variant having at least 80%, 85%, 90%, 95%, 96%, 97%, 98%, or 99% identity with SEQ ID NO: 70.

In some embodiments, a Cas9 protein is a nuclease-inactivated Cas9 protein (also referred to herein as a “dead Cas9” or “dCas9.” In some embodiments, a dCas9 comprises both a D10A and an H840A mutation relative to a wild type Streptococcus pyogenes Cas9 of SEQ ID NO: 70, or a variant having at least 80%, 85%, 90%, 95%, 96%, 97%, 98%, or 99% identity with SEQ ID NO: 70.

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1). Mutations at positions corresponding to D10 and H840 in SEQ ID NO: 70 may also be made at corresponding positions in any of these Cas9 proteins to produce nickase or dead variants.

A “cell type” refers to a classification of cells that share common morphological and/or phenotypical features. Multicellular organisms may be composed of cells of multiple different, specialized cell types (e.g., nervous system cells, immune cells, muscle cells, skin cells, etc.). The ARMMs disclosed herein may, in some embodiments, target one or more than one particular cell type through, for example, the antibody or antigen-binding fragment that is fused to the NiV glycoprotein of the microvesicle. Cell types contemplated for use in the present disclosure include, but are not limited to, stem and progenitor cells (e.g., embryonic stem cells, hematopoietic stem cells, mesenchymal stem cells, neural crest cells, etc.), endothelial cells, muscle cells, myocardial cells, smooth and skeletal muscle cells, mesenchymal cells, epithelial cells, hematopoietic cells, lymphocytes such as T-cells (e.g., Th1 T cells, Th2 T cells, ThO T cells, cytotoxic T cells) and B cells (e.g., pre-B cells), monocytes, dendritic cells, neutrophils, macrophages, natural killer cells, mast cells, adipocytes, immune cells, neurons, hepatocytes, and cells involved with particular organs (e.g., thymus, endocrine glands, pancreas, brain, neurons, glia, astrocytes, dendrocytes, and genetically modified cells thereof). The cells may also be transformed or neoplastic cells of different types (e.g., carcinomas of different cell origins, lymphomas of different cell types, etc.) or cancerous cells of any kind. Cells of different origins (e.g., ectodermal, mesodermal, and endodermal) are also contemplated for use in the present disclosure. In some embodiments, the antibody or antigen-binding fragment of the ARMMs disclosed herein targets a particular cell type. In some embodiments, the cell type is selected from the group consisting of neurons, astrocytes, oligodendrocytes, microglial cells, T-cells, dendritic cells, B cells, NK cells, stem cells, progenitor cells, endothelial cells, muscle cells, myocardial cells, epithelial cells, and hepatic cells. In certain embodiments, the antibody or antigen-binding fragment of the ARMMs disclosed herein targets T-cells. In certain embodiments, the antibody or antigen-binding fragment of the ARMMs disclosed herein targets neurons. Other cell types besides those disclosed herein may be targeted using the presently described ARMMs, and a person of ordinary skill in the art would be able to select antibodies or antigen-binding fragments to target additional cell types besides those specifically listed herein.

A “designed ankyrin repeat protein” or “DARPin” refers to a genetically engineered antibody mimetic protein. DARPins typically exhibit high specificity and high-affinity target protein binding. They are derived from natural ankyrin repeat proteins, which are one of the most common classes of binding proteins in nature and play diverse roles in cell signaling, regulation, and structural integrity. DARPins typically comprise at least three ankyrin repeat motifs. In some embodiments, the antibody or antigen-binding fragment of the microvesicles disclosed herein comprises a DARPin.

As used herein, “expression” of a nucleic acid sequence refers to one or more of the following events: (1) production of an RNA transcript from a DNA sequence (e.g., by transcription); (2) processing of an RNA transcript (e.g., by splicing, editing, 5′ cap formation, and/or 3′ end processing); (3) translation of an RNA transcript into a polypeptide or protein; and (4) post-translational modification of a polypeptide or protein.

As used herein, a “fusion protein” includes a first protein moiety, e.g., an ARRCD1 protein or variant thereof, or a TSG101 protein or variant thereof, associated with a second protein moiety, for example, a protein to be delivered to a target cell through a peptide linkage. In certain embodiments, the fusion protein is encoded by a single fusion gene. It should be noted that the term “fusion protein” can, but need to necessarily, refer to a “NiV fusion protein,” which refers to the Nipah virus surface protein that facilitates viral entry into a target cell and is described further herein. As such, unless used in this context, the term “fusion protein” is distinct from the term “NiV fusion protein.”

As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that the definition of gene includes references to nucleic acids that do not encode proteins but rather encode functional RNA molecules, such as gRNAs, RNAi agents, ribozymes, tRNAs, etc. For the purpose of clarity, it should be noted that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein-coding expression units but rather to clarify that, in most cases, the term as used herein refers to a protein-coding nucleic acid.

As used herein, the term “gene product” or “expression product” generally refers to an RNA transcribed from the gene (pre-and/or post-processing) or a polypeptide (pre-and/or post-modification) encoded by an RNA transcribed from the gene.

The term “linker,” as used herein, refers to a chemical moiety linking two molecules or moieties, e.g., an ARRDC1 protein and a Tat protein, a WW domain and a Tat protein, or an ARRDC1 protein and a Cas9 nuclease, nickase, or dCas9 or other protein or peptide agent as described herein. Typically, the linker is positioned between, or flanked by, two groups, molecules, or other moieties and connected to each one via a covalent bond, thus connecting the two. In some embodiments, the linker comprises an amino acid or a plurality of amino acids (e.g., a peptide or protein). In some embodiments, the linker comprises a nucleotide (e.g., DNA or RNA) or a plurality of nucleotides (e.g., a nucleic acid). In some embodiments, the linker is an organic molecule, functional group, polymer, or other chemical moiety. In some embodiments, the linker is a cleavable linker, e.g., the linker comprises a bond that can be cleaved upon exposure to, for example, UV light or a hydrolytic enzyme, such as a protease or esterase. In some embodiments, the linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids). In other embodiments, the linker is a chemical bond (e.g., a covalent bond, amide bond, disulfide bond, ester bond, carbon-carbon bond, carbon heteroatom bond).

As used herein, the term “microRNA” or “miRNA” refers to an RNAi agent that is approximately 21 nucleotides (nt)-23 nt in length. miRNAs can range between 18 nt-26 nt in length. Typically, miRNAs are single-stranded. However, in some embodiments, miRNAs may be at least partially double-stranded. In certain embodiments, miRNAs may comprise an RNA duplex (referred to herein as a “duplex region”) and may optionally further comprises one to three single-stranded overhangs. In some embodiments, an RNAi agent comprises a duplex region ranging from 15 bp to 29 bp in length and optionally further comprises one or two single-stranded overhangs. A miRNA may be formed from two RNA molecules that hybridize together, or may alternatively be generated from a single RNA molecule that includes a self-hybridizing portion. In general, free 5′ ends of miRNA molecules have phosphate groups, and free 3′ ends have hydroxyl groups. The duplex portion of a miRNA usually, but does not necessarily, comprise one or more bulges consisting of one or more unpaired nucleotides. One strand of a miRNA includes a portion that hybridizes with a target RNA. In certain embodiments, one strand of the miRNA is not precisely complementary with a region of the target RNA, meaning that the miRNA hybridizes to the target RNA with one or more mismatches. In some embodiments, one strand of the miRNA is precisely complementary with a region of the target RNA, meaning that the miRNA hybridizes to the target RNA with no mismatches. Typically, miRNAs are thought to mediate inhibition of gene expression by inhibiting translation of target transcripts. However, in some embodiments, miRNAs may mediate inhibition of gene expression by causing degradation of target transcripts.

The term “microvesicle,” as used herein, refers to a droplet of liquid surrounded by a lipid bilayer. In some embodiments, a microvesicle has a diameter of about 10 nm to about 1000 nm. In some embodiments, a microvesicle has a diameter of at least about 10 nm, at least about 20 nm, at least about 30 nm, at least about 40 nm, at least about 50 nm, at least about 60 nm, at least about 70 nm, at least about 80 nm, at least about 90 nm, at least about 100 nm, at least about 125 nm, at least about 150 nm, at least about 175 nm, at least about 200 nm, at least about 250 nm, at least about 300 nm, at least about 400 nm, or at least about 500 nm. In some embodiments, a microvesicle has a diameter of less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 90 nm, less than about 80 nm, less than about 70 nm, less than about 60 nm, or less than about 50 nm. The term microvesicle includes microvesicles shed from cells as well as synthetically produced microvesicles. Microvesicles shed from cells typically comprise the antigenic content of the cells from which they originate. Microvesicles shed from cells also typically comprise an asymmetric distribution of phospholipids, reflecting the phospholipid distribution of the cells from which they originate. In some embodiments, the inner membrane of microvesicles provided herein, e.g., of some ARMMs, comprises the majority of aminophospholipids, phosphatidylserine, and/or phosphatidylethanolamine within the lipid bilayer.

A “NiV fusion protein” or “NiV-F protein,” as used herein, refers to the fusion protein of the Nipah virus that facilitates fusion of the viral particle to a cell. The wild-type NiV fusion protein comprises the following amino acid sequence:

(SEQ ID NO: 6)
MVVILDKRCYCNLLILILMISECSVGILHYEKLSKIGLVKGVTRK
YKIKSNPLTKDIVIKMIPNVSNMSQCTGSVMENYKTRLNGILTPI
KGALEIYKNNTHDLVGDVRLAGVIMAGVAIGIATAAQITAGVALY
EAMKNADNINKLKSSIESTNEAVVKLQETAEKTVYVLTALQDYIN
TNLVPTIDKISCKQTELSLDLALSKYLSDLLFVFGPNLQDPVSNS
MTIQAISQAFGGNYETLLRTLGYATEDFDDLLESDSITGQIIYVD
LSSYYIIVRVYFPILTEIQQAYIQELLPVSFNNDNSEWISIVPNF
ILVRNTLISNIEIGFCLITKRSVICNQDYATPMTNNMRECLTGST
EKCPRELVVSSHVPRFALSNGVLFANCISVTCQCQTTGRAISQSG
EQTLLMIDNTTCPTAVLGNVIISLGKYLGSVNYNSEGIAIGPPVF
TDKVDISSQISSMNQSLQQSKDYIKEAQRLLDTVNPSLISMLSMI
ILYVLSIASLCIGLITFISFIIVEKKRNTYSRLEDRRVRPTSSGD
LYYIGT.

The sequence of wild-type NiV fusion protein can also be found at https://www.uniprot.org/uniprot/Q9IH63.

In some embodiments, the ARMMs described herein comprise a NiV fusion protein variant that is at least 70%, at least 75%, at least 80%, at least 85%, at least 90%, at least 95%, at least 96%, at least 97%, at least 98%, or at least 99% identical to a wild-type NiV fusion protein of SEQ ID NO: 6. In some embodiments, the ARMMs described herein comprise a NiV fusion protein variant comprising amino acid truncations relative to a wild-type NiV fusion protein of SEQ ID NO: 6.

A “NiV glycoprotein” or “NiV-G protein,” as used herein, refers to the glycoprotein of the Nipah virus that facilitates recognition, binding, and attachment of the virus to a cell. The endogenous targets of the NiV glycoprotein are ephrin receptors (erythropoietin-producing human hepatocellular receptors, the largest known subfamily of receptor tyrosine kinases). The wild-type NiV glycoprotein comprises the following amino acid sequence:

(SEQ ID NO: 1)
MPAENKKVRFENTTSDKGKIPSKVIKSYYGTMDIKKINEGLLDSK
ILSAFNTVIALLGSIVIIVMNIMIIQNYTRSTDNQAVIKDALQGI
QQQIKGLADKIGTEIGPKVSLIDTSSTITIPANIGLLGSKISQST
ASINENVNEKCKFTLPPLKIHECNISCPNPLPFREYRPQTEGVSN
LVGLPNNICLQKTSNQILKPKLISYTLPVVGQSGTCITDPLLAMD
EGYFAYSHLERIGSCSRGVSKQRIIGVGEVLDRGDEVPSLFMTNV
WTPPNPNTVYHCSAVYNNEFYYVLCAVSTVGDPILNSTYWSGSLM
MTRLAVKPKSNGGGYNQHQLALRSIEKGRYDKVMPYGPSGIKQGD
TLYFPAVGFLVRTEFKYNDSNCPITKCQYSKPENCRLSMGIRPNS
HYILRSGLLKYNLSDGENPKVVFIEISDQRLSIGSPSKIYDSLGQ
PVFYQASFSWDTMIKFGDVLTVNPLVVNWRNNTVISRPGQSQCPR
ENTCPEICWEGVYNDAFLIDRINWISAGVFLDSNQTAENPVFTVF
KDNEILYRAQLASEDTNAQKTITNCFLLKNKIWCISLVEIYDTGD
NVIRPKLFAVKIPEQCT

In some embodiments, the ARMMs described herein comprise a NiV glycoprotein variant that is at least 70%, at least 75%, at least 80%, at least 85%, at least 90%, at least 95%, at least 96%, at least 97%, at least 98%, or at least 99% identical to a wild-type NiV glycoprotein of SEQ ID NO: 1. In some embodiments, the ARMMs described herein comprise a NiV glycoprotein variant comprising amino acid substitutions or truncations as described herein. In some embodiments, the ARMMs described herein comprise a NiV glycoprotein variant that does not recognize and/or bind to its natural target (i.e., an ephrin receptor) and that is fused to an antibody or antigen-binding fragment such that the NiV glycoprotein targets the ARMM to a particular cell type.

As used herein, the term “nucleic acid,” in its broadest sense, refers to a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleotides. As used herein, the terms “oligonucleotide” and “polynucleotide” can be used interchangeably to refer to a polymer of nucleotides (e.g., a string of at least two nucleotides). In some embodiments, “nucleic acid” encompasses RNA as well as single and/or double-stranded DNA and/or complementary DNA (cDNA). Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery.

As used herein, the term “protein” refers to a string of at least two amino acids linked to one another by one or more peptide bonds. Proteins may include moieties other than amino acids (e.g., may be glycoproteins) and/or may be otherwise processed or modified. Those of ordinary skill in the art will appreciate that a “protein” can be a complete protein chain as produced by a cell (with or without a signal sequence), or can be a functional portion thereof. Those of ordinary skill will further appreciate that a protein can sometimes include more than one protein chain, for example linked by one or more disulfide bonds or associated by other means. Proteins may contain L-amino acids, D-amino acids, or both and may contain any of a variety of amino acid modifications or analogs known in the art. Useful modifications include, e.g., addition of a chemical entity such as a carbohydrate group, a phosphate group, a farnesyl group, an isofarnesyl group, a fatty acid group, an amide group, a terminal acetyl group, a linker for conjugation, functionalization, or other modification (e.g., alpha amidation), etc. In certain embodiments, the modifications of the protein lead to a more stable protein (e.g., greater half-life in vivo). These modifications may include cyclization of the protein, the incorporation of D-amino acids, etc. None of the modifications should substantially interfere with the desired biological activity of the protein. In certain embodiments, the modifications of the protein lead to a more biologically active protein. In some embodiments, proteins may comprise natural amino acids, non-natural amino acids, synthetic amino acids, amino acid analogs, and combinations thereof.

As used herein, the term “RNA interference” or “RNAi” refers to sequence-specific inhibition of gene expression and/or reduction in target RNA levels mediated by an RNA, which RNA comprises a portion that is substantially complementary to a target RNA. Typically, at least part of the substantially complementary portion is within the double stranded region of the RNA. In some embodiments, RNAi can occur via selective intracellular degradation of RNA. In some embodiments, RNAi can occur by translational repression.

As used herein, the term “RNAi agent” or “RNAi” refers to an RNA, optionally including one or more nucleotide analogs or modifications, having a structure characteristic of molecules that can mediate inhibition of gene expression through an RNAi mechanism. In some embodiments, RNAi agents mediate inhibition of gene expression by causing degradation of target transcripts. In some embodiments, RNAi agents mediate inhibition of gene expression by inhibiting translation of target transcripts. Generally, an RNAi agent includes a portion that is substantially complementary to a target RNA. In some embodiments, RNAi agents are at least partly double-stranded. In some embodiments, RNAi agents are single-stranded. In some embodiments, exemplary RNAi agents can include siRNA, shRNA, and/or miRNA. In some embodiments, RNAi agents may be composed entirely of natural RNA nucleotides (i.e., adenine, guanine, cytosine, and uracil). In some embodiments, RNAi agents may include one or more non-natural RNA nucleotides (e.g., nucleotide analogs, DNA nucleotides, etc.). Inclusion of non-natural RNA nucleic acid residues may be used to make the RNAi agent more resistant to cellular degradation than RNA. In some embodiments, the term “RNAi agent” may refer to any RNA, RNA derivative, and/or nucleic acid encoding an RNA that induces an RNAi effect (e.g., degradation of target RNA and/or inhibition of translation). In some embodiments, an RNAi agent may comprise a blunt-ended (i.e., without overhangs) dsRNA that can act as a Dicer substrate. For example, such an RNAi agent may comprise a blunt-ended dsRNA which is ≥25 base pairs length, which may optionally be chemically modified to abrogate an immune response.

As used herein, the term “short, interfering RNA” or “siRNA” refers to an RNAi agent comprising an RNA duplex (referred to herein as a “duplex region”) that is approximately 19 base pairs (bp) in length and optionally further comprises one to three single-stranded overhangs. In some embodiments, an RNAi agent comprises a duplex region ranging from 15 bp to 29 bp in length and optionally further comprising one or two single-stranded overhangs. An siRNA may be formed from two RNA molecules that hybridize together, or may alternatively be generated from a single RNA molecule that includes a self-hybridizing portion. In general, free 5′-ends of siRNA molecules have phosphate groups, and free 3′-ends have hydroxyl groups. The duplex portion of an siRNA may, but typically does not, comprise one or more bulges consisting of one or more unpaired nucleotides. One strand of an siRNA includes a portion that hybridizes with a target transcript. In certain embodiments, one strand of the siRNA is precisely complementary with a region of the target transcript, meaning that the siRNA hybridizes to the target transcript without a single mismatch. In some embodiments, one or more mismatches between the siRNA and the targeted portion of the target transcript may exist. In some embodiments in which perfect complementarity is not achieved, any mismatches are generally located at or near the siRNA termini. In some embodiments, siRNAs mediate inhibition of gene expression by causing degradation of target transcripts.

As used herein, the term “short hairpin RNA” or “shRNA” refers to an RNAi agent comprising an RNA having at least two complementary portions hybridized or capable of hybridizing to form a double-stranded (duplex) structure sufficiently long to mediate RNAi (typically at least approximately 19 bp in length), and at least one single-stranded portion, typically ranging between approximately 1 nucleotide (nt) and approximately 10 nt in length that forms a loop. In some embodiments, an shRNA comprises a duplex portion ranging from 15 bp to 29 bp in length and at least one single-stranded portion, typically ranging between approximately 1 nt and approximately 10 nt in length that forms a loop. The duplex portion may, but typically does not, comprise one or more bulges consisting of one or more unpaired nucleotides. In some embodiments, siRNAs mediate inhibition of gene expression by causing degradation of target transcripts, shRNAs are thought to be processed into siRNAs by the conserved cellular RNAi machinery. Thus, shRNAs may be precursors of siRNAs. Regardless, siRNAs in general are capable of inhibiting expression of a target RNA, similar to siRNAs.

In general, a “small molecule” refers to a substantially non-peptidic, non-oligomeric organic compound either prepared in the laboratory or found in nature. Small molecules, as used herein, can refer to compounds that are “natural product-like,” however, the term “small molecule” is not limited to “natural product-like” compounds. Rather, a small molecule is typically characterized in that it contains several carbon-carbon bonds and has a molecular weight of less than 2000 g/mol, less than 1500 g/mol, less than 1250 g/mol, less than 1000 g/mol, less than 750 g/mol, less than 500 g/mol, or less than 250 g/mol, although this characterization is not intended to be limiting for the purposes of the present invention. In certain other embodiments, natural-product-like small molecules are utilized.

As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals, such as mice, rats, rabbits, non-human primates, and humans) and/or plants. In some embodiments, the subject is a patient having or suspected of having a disease or disorder. In other embodiments, the subject is a healthy volunteer.

As used herein, the term “therapeutic agent” refers to any agent that, when administered to a subject, has a beneficial effect. In some embodiments, the agent is a small molecule, or a protein or nucleic acid, such as DNA or RNA, that is associated with a small molecule. In some embodiments, the agent to be delivered by the ARMMs described herein is a diagnostic agent. In some embodiments, the agent to be delivered is a prophylactic agent. In some embodiments, the agent to be delivered is useful as an imaging agent. In some of these embodiments, the diagnostic or imaging agent is, and in others it is not, biologically active.

As used herein, the term “therapeutically effective amount” means an amount of an agent to be delivered (e.g., nucleic acid, protein, drug, therapeutic agent, diagnostic agent, prophylactic agent, ARMM, or ARMM comprising a protein agent or RNA agent) that is sufficient, when administered to a subject suffering from or susceptible to a disease, disorder, and/or condition, to treat, improve symptoms of, diagnose, prevent, and/or delay the onset of the disease, disorder, and/or condition. Use of the term “therapeutically effective amount” does not necessarily mean that the amount needs to be, or needs to be shown to be, clinically effective.

As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules. Examples of transcription factors include, but are not limited to, Sp1, NF1, CCAAT, GATA, HNF, PIT-1, MyoD, Myf5, Hox, Winged Helix, SREBP, p53, CREB, AP-1, Mef2, STAT, R-SMAD, NF-κB, Notch, TUBBY, and NFAT.

As used herein, the term “treating” refers to partially or completely preventing, and/or reducing the incidence of one or more symptoms or features of a particular disease or condition. For example, “treating” cancer may refer to inhibiting survival, growth, and/or spread of the cancer. Treatment may be administered to a subject who does not exhibit signs or symptoms of a disease, disorder, and/or condition and/or to a subject who exhibits only early signs or symptoms of a disease, or condition for the purpose of decreasing the risk of developing more severe effects associated with the disease, disorder, or condition. Use of the term “treating” or “treatment” does not necessarily needs to be, or needs to be shown to be, demonstrated clinically.

As used herein, a “vector” means any nucleic acid or nucleic acid-bearing particle, cell, or organism capable of being used to transfer a nucleic acid into a host cell. The term “vector” includes both viral and nonviral products and means for introducing the nucleic acid into a cell. A “vector” can be used in vitro, ex vivo, or in vivo. Vectors capable of directing the expression of operatively linked genes are referred to herein as “expression vectors.” Non-viral vectors include plasmids, cosmids, artificial chromosomes (e.g., bacterial artificial chromosomes or yeast artificial chromosomes) and can comprise liposomes, electrically charged lipids (cytofectins), DNA-protein complexes, and biopolymers, for example. Viral vectors include retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also comprise the entire genome sequence or recombinant genome sequence of a virus. A vector can also comprise a portion of the genome that comprises the functional sequences for production of a virus capable of infecting, entering, or being introduced to a cell to deliver nucleic acid therein.

The term “WW domain,” as used herein, refers to a protein domain having two basic residues at the C-terminus that mediates protein-protein interactions with short proline-rich or proline-containing motifs. It should be appreciated that the two basic residues (e.g., any two of: H, R, and K) of the WW domain are not required to be at the absolute C-terminal end of the WW protein domain. Rather, the two basic residues may be at a C-terminal portion of the WW protein domain (e.g., the C-terminal half of the WW protein domain). In some embodiments, the WW domain contains at least 1, 2, 3, 4, 5, 6, 7, 8, 9, or 10 tryptophan (W) residues. In some embodiments, the WW domain contains at least two W residues. In some embodiments, the at least two W residues are spaced apart by from 15-25 amino acids. In some embodiments, the at least two W residues are spaced apart by from 19-23 amino acids. In some embodiments, the at least two W residues are spaced apart by from 20-22 amino acids. The WW domain possessing the two basic C-terminal amino acid residues may have the ability to associate with short proline-rich or proline-containing motifs (e.g., a PPXY motif). WW domains bind a variety of distinct peptide ligands including motifs with core proline-rich sequences, such as PPXY, which is found in AARDC1. A WW domain may be a 30-40 amino acid protein interaction domain with two signature tryptophan residues spaced by 20-22 amino acids. The three-dimensional structure of WW domains shows that they generally fold into a three-stranded, antiparallel β sheet with two ligand-binding grooves.

WW domains are found in many eukaryotes and are present in approximately 50 human proteins (Bork, P. & Sudol, M. The WW domain: a signaling site in dystrophin? Trends Biochem Sci 19, 531-533 (1994)). WW domains may be present together with several other interaction domains, including membrane targeting domains, such as C2 in the NEDD4 family proteins, the phosphotyrosine-binding (PTB) domain in FE65 protein, FF domains in CA150 and FBPII, and pleckstrin homology (PH) domains in PLEKHA5. WW domains are also linked to a variety of catalytic domains, including HECT E3 protein-ubiquitin ligase domains in NEDD4 family proteins, rotomerase or peptidyl prolyisomerase domains in Pin1, and Rho GAP domains in ArhGAP9 and ArhGAP12.

In the instant disclosure, the WW domain may be a WW domain that naturally possesses two basic amino acids at the C-terminus. In some embodiments, a WW domain or WW domain variant may be from the human ubiquitin ligase WWP1, WWP2, Nedd4-1, Nedd4-2, Smurf1, Smurf2, ITCH, NEDL1, or NEDL2. Exemplary amino acid sequences of WW domain containing proteins (WW domains underlined) are listed below. It should be appreciated that any of the WW domains or WW domain variants of the exemplary proteins may be used in the microvesicles described herein, and the particular WW domains described herein are not meant to be limiting.

Human WWP1 amino acid sequence (uniprot.org/uniprot/Q9H0M0). The four underlined WW domains correspond to amino acids 349-382 (WW1), 381-414 (WW2), 456-489 (WW3), and 496-529 (WW4).

(SEQ ID NO 7)
MATASPRSDT SNNHSGRLQL QVTVSSAKLK RKKNWFGTAI YTEVVVDGEI 50
TKTAKSSSSS NPKWDEQLTV NVTPQTTLEF QVWSHRTLKA DALLGKATID 100
LKQALLIHNR KLERVKEQLK LSLENKNGIA QTGELTVVLD GLVIEQENIT 150
NCSSSPTIEI QENGDALHEN GEPSARTTAR LAVEGTNGID NHVPTSTLVQ 200
NSCCSYVVNG DNTPSSPSQV AARPKNTPAP KPLASEPADD TVNGESSSFA 250
PTDNASVTGT PVVSEENALS PNCTSTTVED PPVQEILTSS ENNECIPSTS 300
AELESEARSI LEPDTSNSRS SSAFEAAKSR QPDGCMDPVR QQSGNANTET 350
LPSGWEQRKD PHGRTYYVDH NTRTTTWERP QPLPPGWERR VDDRRRVYYV 400
DHNTRTTTWQ RPTMESVRNF EQWQSQRNQL QGAMQQFNQR YLYSASMLAA 450
ENDPYGPLPP GWEKRVDSTD RVYFVNHNTK TTQWEDPRTQ GLQNEEPLPE 500
GWEIRYTREG VRYFVDHNTR TTTEKDPRNG KSSVTKGGPQ IAYERGFRWK 550
LAHFRYLCQS NALPSHVKIN VSRQTLFEDS FQQIMALKPY DLRRRLYVIF 600
RGEEGLDYGG LAREWFFLLS HEVLNPMYCL FEYAGKNNYC LQINPASTIN 650
PDHLSYFCFI GRFIAMALFH GKFIDTGFSL PFYKRMLSKK LTIKDLESID 700
TEFYNSLIWI RDNNIEECGL EMYFSVDMEI LGKVTSHDLK LGGSNILVTE 750
ENKDEYIGLM TEWRFSRGVQ EQTKAFLDGF NEVVPLQWLQ YEDEKELEVM 800
LCGMQEVDLA DWQRNTVYRH YTRNSKQIIW FWQFVKETDN EVRMRLLQFV 850
TGTCRLPLGG FAELMGSNGP QKFCIEKVGK DTWLPRSHTC FNRLDLPPYK 900
SYEQLKEKLL FAIEETEGFG QE 922
WW1 (349-382):
(SEQ ID NO: 8)
ETLPSGWEQRKDPHGRTYYVDHNTRTTTWERPQP.
WW2 (381-414):
(SEQ ID NO: 9)
QPLPPGWERRVDDRRRVYYVDHNTRTTTWQRPTM.
WW3 (456-489):
(SEQ ID NO: 10)
ENDPYGPLPPGWEKRVDSTDRVYFVNHNTKTTQWEDPRT.
WW4 (496-529):
(SEQ ID NO: 11)
EPLPEGWEIRYTREGVRYFVDHNTRTTTFKDPRN

Human WWP2 amino acid sequence (uniprot.org/uniprot/O00308). The four underlined WW domains correspond to amino acids 300-333 (WW1), 330-363 (WW2), 405-437 (WW3), and 444-547 (WW4).

(SEQ ID NO: 12)
MASASSSRAG VALPFEKSQL TLKVVSAKPK VHNRQPRINS YVEVAVDGLP 50
SETKKTGKRI GSSELLWNEI IILNVTAQSH LDLKVWSCHT LRNELLGTAS 100
VNLSNVLKNN GGKMENMQLT LNLQTENKGS VVSGGELTIF LDGPTVDLGN 150
VPNGSALTDG SQLPSRDSSG TAVAPENRHQ PPSTNCFGGR SRTHRHSGAS 200
ARTTPATGEQ SPGARSRHRQ PVKNSGHSGL ANGTVNDEPT TATDPEEPSV 250
VGVTSPPAAP LSVTPNPNTT SLPAPATPAE GEEPSTSGTQ QLPAAAQAPD 300
ALPAGWEQRE LPNGRVYYVD HNTKTTTWER PLPPGWEKRT DPRGRFYYVD 350
HNTRTTTWQR PTAEYVRNYE QWQSQRNQLQ GAMQHFSQRF LYQSSSASTD 400
HDPLGPLPPG WEKRQDNGRV YYVNHNTRTT QWEDPRTQGM IQEPALPPGW 450
EMKYTSEGVR YFVDHNTRTT TFKDPRPGFE SGTKQGSPGA YDRSFRWKYH 500
QFRFLCHSNA LPSHVKISVS RQTLFEDSFQ QIMNMKPYDL RRRLYIIMRG 550
EEGLDYGGIA REWFFLLSHE VLNPMYCLFE YAGKNNYCLQ INPASSINPD 600
HLTYFRFIGR FIAMALYHGK FIDTGFTLPF YKRMLNKRPT LKDLESIDPE 650
FYNSIVWIKE NNLEECGLEL YFIQDMEILG KVTTHELKEG GESIRVTEEN 700
KEEYIMLLTD WRFTRGVEEQ TKAFLDGFNE VAPLEWLRYF DEKELELMLC 750
GMQEIDMSDW QKSTIYRHYT KNSKQIQWFW QVVKEMDNEK RIRLLQFVTG 800
TCRLPVGGFA ELIGSNGPQK FCIDKVGKET WLPRSHTCFN RLDLPPYKSY 850
EQLREKLLYA IEETEGFGQE 870
WW1 (300-333):
(SEQ ID NO: 13)
DALPAGWEQRELPNGRVYYVDHNTKTTTWERPLP.
WW2 (330-363):
(SEQ ID NO: 14)
PLPPGWEKRTDPRGRFYYVDHNTRTTTWQRPTA.
WW3 (405-437):
(SEQ ID NO: 15)
HDPLGPLPPGWEKRQDNGRVYYVNHNTRTTQWEDPRT.
WW4 (444-477):
(SEQ ID NO: 16)
PALPPGWEMKYTSEGVRYFVDHNTRTTTFKDPRP.

Human Nedd4-1 amino acid sequence (uniprot.org/uniprot/P46934). The four underlined WW domains correspond to amino acids 610-643 (WW1), 767-800 (WW2), 840-873 (WW3), and 892-925 (WW4).

(SEQ ID NO: 17)
MAQSLRLHFA ARRSNTYPLS ETSGDDLDSH VHMCFKRPTR ISTSNVVQMK 50
LTPRQTALAP LIKENVQSQE RSSVPSSENV NKKSSCLQIS LQPTRYSGYL 100
QSSNVLADSD DASFTCILKD GIYSSAVVDN ELNAVNDGHL VSSPAICSGS 150
LSNFSTSDNG SYSSNGSDFG SCASITSGGS YTNSVISDSS SYTFPPSDDT 200
FLGGNLPSDS TSNRSVPNRN TTPCEIFSRS TSTDPFVQDD LEHGLEIMKL 250
PVSRNTKIPL KRYSSLVIFP RSPSTTRPTS PTSLCTLLSK GSYQTSHQFI 300
ISPSEIAHNE DGTSAKGFLS TAVNGLRLSK TICTPGEVRD IRPLHRKGSL 350
QKKIVLSNNT PRQTVCEKSS EGYSCVSVHF TQRKAATLDC ETTNGDCKPE 400
MSEIKLNSDS EYIKLMHRTS ACLPSSQNVD CQININGELE RPHSQMNKNH 450
GILRRSISLG GAYPNISCLS SLKHNCSKGG PSQLLIKFAS GNEGKVDNLS 500
RDSNRDCTNE LSNSCKTRDD FLGQVDVPLY PLPTENPRLE RPYTFKDFVL 550
HPRSHKSRVK GYLRLKMTYL PKTSGSEDDN AEQAEELEPG WVVLDQPDAA 600
CHLQQQQEPS PLPPGWEERQ DILGRTYYVN HESRRTQWKR PTPQDNLTDA 650
ENGNIQLQAQ RAFTTRRQIS EETESVDNRE SSENWEIIRE DEATMYSNQA 700
FPSPPPSSNL DVPTHLAEEL NARLTIFGNS AVSQPASSSN HSSRRGSLQA 750
YTFEEQPTLP VLLPTSSGLP PGWEEKQDER GRSYYVDHNS RTTTWTKPTV 800
QATVETSQLT SSQSSAGPQS QASTSDSGQQ VTQPSEIEQG FLPKGWEVRH 850
APNGRPFFID HNTKTTTWED PRLKIPAHLR GKTSLDTSND LGPLPPGWEE 900
RTHTDGRIFY INHNIKRTQW EDPRLENVAI TGPAVPYSRD YKRKYEFFRR 950
KLKKQNDIPN KFEMKLRRAT VLEDSYRRIM GVKRADFLKA RLWIEFDGEK 1000
GLDYGGVARE WFFLISKEMF NPYYGLFEYS ATDNYTLQIN PNSGLCNEDH 1050
LSYFKFIGRV AGMAVYHGKL LDGFFIRPFY KMMLHKPITL HDMESVDSEY 1100
YNSLRWILEN DPTELDLRFI IDEELFGQTH QHELKNGGSE IVVINKNKKE 1150
YIYLVIQWRF VNRIQKQMAA FKEGFFELIP QDLIKIFDEN ELELLMCGLG 1200
DVDVNDWREH TKYKNGYSAN HQVIQWFWKA VLMMDSEKRI RLLQFVTGTS 1250
RVPMNGFAEL YGSNGPQSFT VEQWGTPEKL PRAHTCFNRL DLPPYESFEE 1300
LWDKLQMAIE NTQGFDGVD 1319
WW1(610-643):
(SEQ ID NO: 18)
SPLPPGWEERQDILGRTYYVNHESRRTQWKRPTP.
WW2 (767-800):
(SEQ ID NO: 19)
SGLPPGWEEKQDERGRSYYVDHNSRTTTWTKPTV.
WW3 (840-873):
(SEQ ID NO: 20)
GFLPKGWEVRHAPNGRPFFIDHNTKTTTWEDPRL.
WW4 (892-925):
(SEQ ID NO: 21)
GPLPPGWEERTHTDGRIFYINHNIKRTQWEDPRL.

Human Nedd4-2 amino acid sequence (>gi|21361472|ref|NP_056092.2|E3 ubiquitin-protein ligase NEDD4-like isoform 3 [Homo sapiens]). The four underlined WW domains correspond to amino acids 198-224 (WW1), 368-396 (WW2), 480-510 (WW3), and 531-561 (WW4).

(SEQ ID NO: 22)
MATGLGEPVYGLSEDEGESRILRVKVVSGIDLAKKDIFGASDPYV
KLSLYVADENRELALVQTKTIKKTLNPKWNEEFYFRVNPSNHRLL
FEVFDENRLTRDDFLGQVDVPLSHLPTEDPTMERPYTFKDFLLRP
RSHKSRVKGFLRLKMAYMPKNGGQDEENSDQRDDMEHGWEVVDSN
DSASQHQEELPPPPLPPGWEEKVDNLGRTYYVNHNNRTTQWHRPS
LMDVSSESDNNIRQINQEAAHRRFRSRRHISEDLEPEPSEGGDVP
EPWETISEEVNIAGDSLGLALPPPPASPGSRTSPQELSEELSRRL
QITPDSNGEQFSSLIQREPSSRLRSCSVTDAVAEQGHLPPPSVAY
VHTTPGLPSGWEERKDAKGRTYYVNHNNRTTTWTRPIMQLAEDGA
SGSATNSNNHLIEPQIRRPRSLSSPTVTLSAPLEGAKDSPVRRAV
KDTLSNPQSPQPSPYNSPKPQHKVTQSFLPPGWEMRIAPNGRPFF
IDHNTKTTTWEDPRLKFPVHMRSKTSLNPNDLGPLPPGWEERIHL
DGRTFYIDHNSKITQWEDPRLQNPAITGPAVPYSREFKQKYDYFR
KKLKKPADIPNRFEMKLHRNNIFEESYRRIMSVKRPDVLKARLWI
EFESEKGLDYGGVAREWFFLLSKEMFNPYYGLFEYSATDNYTLQI
NPNSGLCNEDHLSYFTFIGRVAGLAVFHGKLLDGFFIRPFYKMML
GKQITLNDMESVDSEYYNSLKWILENDPTELDLMFCIDEENFGQT
YQVDLKPNGSEIMVTNENKREYIDLVIQWRFVNRVQKQMNAFLEG
FTELLPIDLIKIFDENELELLMCGLGDVDVNDWRQHSIYKNGYCP
NHPVIQWFWKAVLLMDAEKRIRLLQFVTGTSRVPMNGFAELYGSN
GPQLFTIEQWGSPEKLPRAHTCFNRLDLPPYETFEDLREKLLMAV
ENAQGFEGVD
WW1(198-224):
(SEQ ID NO: 23)
GWEEKVDNLGRTYYVNHNNRTTQWHRP.
WW2 (368-396):
(SEQ ID NO: 24)
PSGWEERKDAKGRTYYVNHNNRTTTWTRP.
WW3 (480-510):
(SEQ ID NO: 25)
PPGWEMRIAPNGRPFFIDHNTKTTTWEDPRL.
WW4 (531-561):
(SEQ ID NO: 26)
PPGWEERIHLDGRTFYIDHNSKITQWEDPRL.

Human Smurf1 amino acid sequence (uniprot.org/uniprot/Q9HCE7). The two underlined WW domains correspond to amino acids 234-267 (WW1) and 306-339 (WW2).

(SEQ ID NO: 27)
MSNPGTRRNG SSIKIRLTVL CAKNLAKKDF FRLPDPFAKI VVDGSGQCHS 50
TDTVKNTLDP KWNQHYDLYV GKTDSITISV WNHKKIHKKQ GAGFLGCVRL 100
LSNAISRLKD TGYQRLDLCK LNPSDTDAVR GQIVVSLQTR DRIGTGGSVV 150
DCRGLLENEG TVYEDSGPGR PLSCFMEEPA PYTDSTGAAA GGGNCRFVES 200
PSQDQRLQAQ RLRNPDVRGS LQTPQNRPHG HQSPELPEGY EQRTTVQGQV 250
YFLHTQTGVS TWHDPRIPSP SGTIPGGDAA FLYEFLLQGH TSEPRDLNSV 300
NCDELGPLPP GWEVRSTVSG RIYFVDHNNR TTQFTDPRLH HIMNHQCQLK 350
EPSQPLPLPS EGSLEDEELP AQRYERDLVQ KLKVLRHELS LQQPQAGHCR 400
IEVSREEIFE ESYRQIMKMR PKDLKKRLMV KFRGEEGLDY GGVAREWLYL 450
LCHEMLNPYY GLFQYSTDNI YMLQINPDSS INPDHLSYFH FVGRIMGLAV 500
FHGHYINGGF TVPFYKQLLG KPIQLSDLES VDPELHKSLV WILENDITPV 550
LDHTFCVEHN AFGRILQHEL KPNGRNVPVT EENKKEYVRL YVNWRFMRGI 600
EAQFLALQKG FNELIPQHLL KPFDQKELEL IIGGLDKIDL NDWKSNTRLK 650
HCVADSNIVR WFWQAVETFD EERRARLLQF VTGSTRVPLQ GFKALQGSTG 700
AAGPRLFTIH LIDANTDNLP KAHTCFNRID IPPYESYEKL YEKLLTAVEE 750
TCGFAVE 757
WW1 (234-267):
(SEQ ID NO: 28)
PELPEGYEQRTTVQGQVYFLHTQTGVSTWHDPRI.
WW2 (306-339):
(SEQ ID NO: 29)
GPLPPGWEVRSTVSGRIYFVDHNNRTTQFTDPRL

Human Smurf2 amino acid sequence (uniprot.org/uniprot/Q9HAU4). The three underlined WW domains correspond to amino acids 157-190 (WW1), 251-284 (WW2), and 297-330 (WW3).

(SEQ ID NO: 30)
MSNPGGRRNG PVKLRLTVLC AKNLVKKDFF RLPDPFAKVV VDGSGQCHST 50
DTVKNTLDPK WNQHYDLYIG KSDSVTISVW NHKKIHKKQG AGFLGCVRLL 100
SNAINRLKDT GYQRLDLCKL GPNDNDTVRG QIVVSLQSRD RIGTGGQVVD 150
CSRLFDNDLP DGWEERRTAS GRIQYLNHIT RTTQWERPTR PASEYSSPGR 200
PLSCFVDENT PISGTNGATC GQSSDPRLAE RRVRSQRHRN YMSRTHLHTP 250
PDLPEGYEQR TTQQGQVYFL HTQTGVSTWH DPRVPRDLSN INCEELGPLP 300
PGWEIRNTAT GRVYFVDHNN RTTQFTDPRL SANLHLVLNR QNQLKDQQQQ 350
QVVSLCPDDT ECLTVPRYKR DLVQKLKILR QELSQQQPQA GHCRIEVSRE 400
EIFEESYRQV MKMRPKDLWK RLMIKFRGEE GLDYGGVARE WLYLLSHEML 450
NPYYGLFQYS RDDIYTLQIN PDSAVNPEHL SYFHFVGRIM GMAVFHGHYI 500
DGGFTLPFYK QLLGKSITLD DMELVDPDLH NSLVWILEND ITGVLDHTFC 550
VEHNAYGEII QHELKPNGKS IPVNEENKKE YVRLYVNWRF LRGIEAQFLA 600
LQKGFNEVIP QHLLKTFDEK ELELIICGLG KIDVNDWKVN TRLKHCTPDS 650
NIVKWFWKAV EFFDEERRAR LLQFVTGSSR VPLQGFKALQ GAAGPRLFTI 700
HQIDACTNNL PKAHTCFNRI DIPPYESYEK LYEKLLTAIE ETCGFAVE 748
WW1 (157-190):
(SEQ ID NO: 31)
NDLPDGWEERRTASGRIQYLNHITRTTQWERPTR.
WW2 (251-284):
(SEQ ID NO: 32)
PDLPEGYEQRTTQQGQVYFLHTQTGVSTWHDPRV.
WW3 (297-330):
(SEQ ID NO: 33)
GPLPPGWEIRNTATGRVYFVDHNNRTTQFTDPRL.

Human ITCH amino acid sequence (uniprot.org/uniprot/Q96J02). The four underlined WW domains correspond to amino acids 326-359 (WW1), 358-391 (WW2), 438-471 (WW3), and 478-511 (WW4).

(SEQ ID NO: 34)
MSDSGSQLGS MGSLTMKSQL QITVISAKLK ENKKNWFGPS PYVEVTVDGQ 50
SKKTEKCNNT NSPKWKQPLT VIVTPVSKLH FRVWSHQTLK SDVLLGTAAL 100
DIYETLKSNN MKLEEVVVTL QLGGDKEPTE TIGDLSICLD GLQLESEVVT 150
NGETTCSENG VSLCLPRLEC NSAISAHCNL CLPGLSDSPI SASRVAGFTG 200
ASQNDDGSRS KDETRVSTNG SDDPEDAGAG ENRRVSGNNS PSLSNGGFKP 250
SRPPRPSRPP PPTPRRPASV NGSPSATSES DGSSTGSLPP TNTNTNTSEG 300
ATSGLIIPLT ISGGSGPRPL NPVTQAPLPP GWEQRVDQHG RVYYVDHVEK 350
RTTWDRPEPL PPGWERRVDN MGRIYYVDHF TRTTTWQRPT LESVRNYEQW 400
QLQRSQLQGA MQQFNQRFIY GNQDLFATSQ SKEFDPLGPL PPGWEKRTDS 450
NGRVYFVNHN TRITQWEDPR SQGQLNEKPL PEGWEMRFTV DGIPYFVDHN 500
RRTTTYIDPR TGKSALDNGP QIAYVRDFKA KVQYFRFWCQ QLAMPQHIKI 550
TVTRKTLFED SFQQIMSFSP QDLRRRLWVI FPGEEGLDYG GVAREWFFLL 600
SHEVLNPMYC LFEYAGKDNY CLQINPASYI NPDHLKYFRF IGRFIAMALF 650
HGKFIDTGFS LPFYKRILNK PVGLKDLESI DPEFYNSLIW VKENNIEECD 700
LEMYFSVDKE ILGEIKSHDL KPNGGNILVT EENKEEYIRM VAEWRLSRGV 750
EEQTQAFFEG FNEILPQQYL QYFDAKELEV LLCGMQEIDL NDWQRHAIYR 800
HYARTSKQIM WFWQFVKEID NEKRMRLLQF VTGTCRLPVG GFADLMGSNG 850
PQKFCIEKVG KENWLPRSHT CFNRLDLPPY KSYEQLKEKL LFAIEETEGF 900
GQE 903
ITCH WW1 (326-359):
(SEQ ID NO: 35)
APLPPGWEQRVDQHGRVYYVDHVEKRTTWDRPEP.
ITCH WW2 (358-391):
(SEQ ID NO: 36)
EPLPPGWERRVDNMGRIYYVDHFTRTTTWQRPTL.
ITCH WW3 (438-471):
(SEQ ID NO: 37)
GPLPPGWEKRTDSNGRVYFVNHNTRITQWEDPRS.
ITCH WW4 (478-511):
(SEQ ID NO: 38)
KPLPEGWEMRFTVDGIPYFVDHNRRTTTYIDPRT.

Human NEDL1 amino acid sequence (uniprot.org/uniprot/Q76N89). The two underlined WW domains correspond to amino acids 829-862 (WW1), and 1018-1051 (WW2).

(SEQ ID NO: 39)
MLLHLCSVKN LYQNRFLGLA AMASPSRNSQ SRRRCKEPLR YSYNPDQFHN 50
MDLRGGPHDG VTIPRSTSDT DLVTSDSRST LMVSSSYYSI GHSQDLVIHW 100
DIKEEVDAGD WIGMYLIDEV LSENFLDYKN RGVNGSHRGQ IIWKIDASSY 150
FVEPETKICF KYYHGVSGAL RATTPSVTVK NSAAPIFKSI GADETVQGQG 200
SRRLISESLS DFQAMGLKKG MFFNPDPYLK ISIQPGKHSI FPALPHHGQE 250
RRSKIIGNTV NPIWQAEQFS FVSLPTDVLE IEVKDKFAKS RPIIKRFLGK 300
LSMPVQRLLE RHAIGDRVVS YTLGRRLPTD HVSGQLQFRF EITSSIHPDD 350
EEISLSTEPE SAQIQDSPMN NLMESGSGEP RSEAPESSES WKPEQLGEGS 400
VPDGPGNQSI ELSRPAEEAA VITEAGDQGM VSVGPEGAGE LLAQVQKDIQ 450
PAPSAEELAE QLDLGEEASA LLLEDGEAPA STKEEPLEEE ATTQSRAGRE 500
EEEKEQEEEG DVSTLEQGEG RLQLRASVKR KSRPCSLPVS ELETVIASAC 550
GDPETPRTHY IRIHTLLHSM PSAQGGSAAE EEDGAEEEST LKDSSEKDGL 600
SEVDTVAADP SALEEDREEP EGATPGTAHP GHSGGHEPSL ANGAAQDGDT 650
HPSTGSESDS SPRQGGDHSC EGCDASCCSP SCYSSSCYST SCYSSSCYSA 700
SCYSPSCYNG NRFASHTRES SVDSAKISES TVFSSQDDEE EENSAFESVP 750
DSMQSPELDP ESTNGAGPWQ DELAAPSGHV ERSPEGLESP VAGPSNRREG 800
ECPILHNSQP VSQLPSLRPE HHHYPTIDEP LPPNWEARID SHGRVFYVDH 850
VNRTTTWQRP TAAATPDGMR RSGSIQQMEQ LNRRYQNIQR TIATERSEED 900
SGSQSCEQAP AGGGGGGGSD SEAESSQSSL DLRREGSLSP VNSQKITLLL 950
QSPAVKFITN PEFFTVLHAN YSAYRVFTSS TCLKHMILKV RRDARNFERY 1000
QHNRDLVNFI NMFADTRLEL PRGWEIKTDQ QGKSFFVDHN SRATTFIDPR 1050
IPLQNGRLPN HLTHRQHLQR LRSYSAGEAS EVSRNRGASL LARPGHSLVA 1100
AIRSQHQHES LPLAYNDKIV AFLRQPNIFE MLQERQPSLA RNHTLREKIH 1150
YIRTEGNHGL EKLSCDADLV ILLSLFEEEI MSYVPLQAAF HPGYSFSPRC 1200
SPCSSPQNSP GLQRASARAP SPYRRDFEAK LRNFYRKLEA KGFGQGPGKI 1250
KLIIRRDHLL EGTFNQVMAY SRKELQRNKL YVTFVGEEGL DYSGPSREFF 1300
FLLSQELFNP YYGLFEYSAN DTYTVQISPM SAFVENHLEW FRFSGRILGL 1350
ALIHQYLLDA FFTRPFYKAL LRLPCDLSDL EYLDEEFHQS LQWMKDNNIT 1400
DILDLTFTVN EEVFGQVTER ELKSGGANTQ VTEKNKKEYI ERMVKWRVER 1450
GVVQQTEALV RGFYEVVDSR LVSVFDAREL ELVIAGTAEI DLNDWRNNTE 1500
YRGGYHDGHL VIRWFWAAVE RENNEQRLRL LQFVTGTSSV PYEGFAALRG 1550
SNGLRRFCIE KWGKITSLPR AHTCFNRLDL PPYPSYSMLY EKLLTAVEET 1600
STEGLE 1606
WW1(829-862):
(SEQ ID NO: 40)
PLPPNWEARIDSHGRVFYVDHVNRTTTWQRPTA.
WW2 (1018-1051):
(SEQ ID NO: 41)
LELPRGWEIKTDQQGKSFFVDHNSRATTFIDPRI.

Human NEDL2 amino acid sequence (uniprot.org/uniprot/Q9P2P5). The two underlined WW domains correspond to amino acids 807-840 (WW1) and 985-1018 (WW2).

(SEQ ID NO: 42)
MASSAREHLL FVRRRNPQMR YTLSPENLQS LAAQSSMPEN MTLQRANSDT 50
DLVTSESRSS LTASMYEYTL GQAQNLIIFW DIKEEVDPSD WIGLYHIDEN 100
SPANFWDSKN RGVTGTQKGQ IVWRIEPGPY FMEPEIKICF KYYHGISGAL 150
RATTPCITVK NPAVMMGAEG MEGGASGNLH SRKLVSFTLS DLRAVGLKKG 200
MFFNPDPYLK MSIQPGKKSS FPTCAHHGQE RRSTIISNTT NPIWHREKYS 250
FFALLTDVLE IEIKDKFAKS RPIIKRFLGK LTIPVQRLLE RQAIGDQMLS 300
YNLGRRLPAD HVSGYLQFKV EVTSSVHEDA SPEAVGTILG VNSVNGDLGS 350
PSDDEDMPGS HHDSQVCSNG PVSEDSAADG TPKHSFRTSS TLEIDTEELT 400
STSSRTSPPR GRQDSLNDYL DAIEHNGHSR PGTATCSERS MGASPKLRSS 450
FPTDTRLNAM LHIDSDEEDH EFQQDLGYPS SLEEEGGLIM FSRASRADDG 500
SLTSQTKLED NPVENEEAST HEAASFEDKP ENLPELAESS LPAGPAPEEG 550
EGGPEPQPSA DQGSAELCGS QEVDQPTSGA DTGTSDASGG SRRAVSETES 600
LDQGSEPSQV SSETEPSDPA RTESVSEAST RPEGESDLEC ADSSCNESVT 650
TQLSSVDTRC SSLESARFPE TPAFSSQEEE DGACAAEPTS SGPAEGSQES 700
VCTAGSLPVV QVPSGEDEGP GAESATVPDQ EELGEVWQRR GSLEGAAAAA 750
ESPPQEEGSA GEAQGTCEGA TAQEEGATGG SQANGHQPLR SLPSVRQDVS 800
RYQRVDEALP PNWEARIDSH GRIFYVDHVN RTTTWQRPTA PPAPQVLQRS 850
NSIQQMEQLN RRYQSIRRTM TNERPEENTN AIDGAGEEAD FHQASADFRR 900
ENILPHSTSR SRITLLLQSP PVKFLISPEF FTVLHSNPSA YRMFTNNTCL 950
KHMITKVRRD THHFERYQHN RDLVGFLNMF ANKQLELPRG WEMKHDHQGK 1000
AFFVDHNSRT TTFIDPRLPL QSSRPTSALV HRQHLTRQRS HSAGEVGEDS 1050
RHAGPPVLPR PSSTFNTVSR PQYQDMVPVA YNDKIVAFLR QPNIFEILQE 1100
RQPDLTRNHS LREKIQFIRT EGTPGLVRLS SDADLVMLLS LFEEEIMSYV 1150
PPHALLHPSY CQSPRGSPVS SPQNSPGTQR ANARAPAPYK RDFEAKLRNF 1200
YRKLETKGYG QGPGKLKLII RRDHLLEDAF NQIMGYSRKD LQRNKLYVTF 1250
VGEEGLDYSG PSREFFFLVS RELFNPYYGL FEYSANDTYT VQISPMSAFV 1300
DNHHEWFRFS GRILGLALIH QYLLDAFFTR PFYKALLRIL CDLSDLEYLD 1350
EEFHQSLQWM KDNDIHDILD LTFTVNEEVF GQITERELKP GGANIPVTEK 1400
NKKEYIERMV KWRIERGVVQ QTESLVRGFY EVVDARLVSV FDARELELVI 1450
AGTAEIDLSD WRNNTEYRGG YHDNHIVIRW FWAAVERFNN EQRLRLLQFV 1500
TGTSSIPYEG FASLRGSNGP RRFCVEKWGK ITALPRAHTC FNRLDLPPYP 1550
SFSMLYEKLL TAVEETSTFG LE 1572
WW1 (807-840):
(SEQ ID NO: 43)
EALPPNWEARIDSHGRIFYVDHVNRTTTWQRPTA.
WW2 (985-1018):
(SEQ ID NO: 44)
LELPRGWEMKHDHQGKAFFVDHNSRTTTFIDPRL.

In some embodiments, the WW domain consists essentially of a WW domain or WW domain variant. Consists essentially of means that a domain, peptide, or polypeptide consists essentially of an amino acid sequence when such an amino acid sequence is present with only a few additional amino acid residues, for example, from about 1 to about 10 or so additional residues, typically from 1 to about 5 additional residues in the domain, peptide, or polypeptide.

Alternatively, the WW domain may be a WW domain that has been modified to include two basic amino acids at the C-terminus of the domain. Techniques are known in the art and are described in the art, for example, in Sambrook et al., ((2001) Molecular Cloning: a Laboratory Manual, 3rd ed., Cold Spring Harbour Laboratory Press). Thus, a skilled person could readily modify an existing WW domain that does not normally have two C-terminal basic residues so as to include two basic residues at the C-terminus.

Basic amino acids are amino acids that possess a side-chain functional group that has a pKa of greater than 7 and include lysine, arginine, and histidine, as well as basic amino acids that are not included in the twenty α-amino acids commonly included in proteins. The two basic amino acids at the C-terminus of the WW domain may be the same basic amino acid or may be different basic amino acids. In one embodiment, the two basic amino acids are two arginines.

The term WW domain also includes variants of a WW domain, provided that any such variant possesses two basic amino acids at its C-terminus and maintains the ability of the WW domain to associate with the PPXY motif. A variant of such a WW domain refers to a WW domain that retains the ability of the variant to associate with the PPXY motif (i.e., the PPXY motif of ARRDC1 and that has been mutated at one or more amino acids, including point, insertion, and/or deletion mutations, but still retains the ability to associate with the PPXY motif. A variant or derivative therefore includes deletions, including truncations and fragments; insertions and additions, for example conservative substitutions, site-directed mutants, and allelic variants; and modifications, including one or more non-amino acyl groups (e.g., sugar, lipid, etc.) covalently linked to the peptide and post-translational modifications. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

The WW domain may be part of a longer protein. Thus, the protein, in various different embodiments, comprises the WW domain, consists of the WW domain, or consists essentially of the WW domain, as defined herein. The polypeptide may be a protein that includes a WW domain as a functional domain within the protein sequence.

### DETAILED DESCRIPTION OF CERTAIN EMBODIMENTS

The instant disclosure relates, at least in part, to the discovery that an agent (e.g., a therapeutic agent, or any of the agents described herein) can be delivered to a particular target cell type using an ARMM that has been modified with a NiV glycoprotein and NiV fusion protein. Such modifications allow for the targeted delivery of agents contained in the microvesicles into specific cells. ARMMs modified with a NiV glycoprotein that has been fused to an antibody or antigen-binding fragment, such as an scFv or antibody mimetic protein, can deliver agents such as proteins and RNAs to cells in a targeted manner. NiV glycoproteins can also be fused to other proteins that can deliver the ARMMs to cells in a targeted manner. Various agents, including proteins such as Cas9 and/or nucleic acids such as DNA and/or RNA, can be loaded in such ARMMs for targeted delivery. Various types of protein agents, nucleic acid agents, and other agents are known in the art and include those described in U.S. Pat. Nos. 9,737,480; 9,816,080; 10,260,055; and PCT Application Publication WO2018/067546; the entire contents of each of which are incorporated by reference herein.

Thus, in various aspects, the present disclosure provides arrestin domain-containing protein 1 (ARRDC1)-mediated microvesicles (ARMMs) modified with Nipah virus (NiV) glycoproteins fused to antibodies or antigen-binding fragments and NiV fusion proteins, and variants thereof. The ARMMs of the present disclosure may be targeted to particular cell types and used to deliver agents (e.g., therapeutic agents) to target cells. Microvesicle-producing cells are also provided by the present disclosure. The present disclosure also provides methods of delivering molecules to target cells using the ARMMs described herein, and methods of treating a patient using any of the ARMMs or microvesicle-producing cells provided herein. Kits comprising any of the ARMMs or microvesicle-producing cells described herein are also provided by the present disclosure.

### ARMMs

In some aspects, the present disclosure relates to arrestin domain-containing protein 1 (ARRDC1)-mediated microvesicles (ARMMs). Such ARMMs typically include a lipid bilayer and an ARRDC1 protein or a variant thereof. ARMMs are extracellular vesicles (EVs) that are distinct from exosomes. The budding of ARMMs requires Arrestin domain containing protein 1 (ARRDC1), which is localized to the cytosolic side of the plasma membrane and, through a tetrapeptide motif, recruits the ESCRT-I complex protein TSG101 to the cell surface to initiate the outward membrane budding. Thus, in contrast to exosomes, the biogenesis of ARMMs occurs at the plasma membrane. ARMMs exhibit several additional features that make them potentially ideal vehicles for therapeutic delivery. ARRDC1 is not only necessary, but also sufficient to drive ARMMs budding. Indeed, simple overexpression of the ARRDC1 protein increases the production of ARMMs in cells. This allows controlled production of ARMMs using modern biological manufacturing methods. Moreover, endogenous proteins such as cell surface receptors are actively recruited into ARMMs and can be delivered into recipient cells to initiate intercellular communication, and exogenous agents (e.g., any of the agents described herein, including therapeutic agents) may similarly be delivered via ARMMs.

In particular, the present disclosure relates to ARMMs comprising: (i) a lipid bilayer and an ARRDC1 protein, (ii) a Nipah virus (NiV) glycoprotein variant fused to an antibody or antigen-binding fragment, wherein the NiV glycoprotein variant does not recognize and/or bind to an ephrin receptor, and (iii) a NiV fusion protein. As described herein, surface modification of ARMMs with the NiV glycoprotein and fusion protein allows targeted delivery of agents encapsulated in the vesicles into specific cells.

### ARRDC1

ARRDC1 is a protein that comprises a PSAP (SEQ ID NO: 2) motif and a PPXY motif in its C-terminus and interacts with TSG101. It should be appreciated that the PSAP (SEQ ID NO: 2) motif and the PPXY motif are not required to be at the absolute C-terminal end of the ARRDC1. Rather, they may be at a C-terminal portion of the ARRDC1 protein (e.g., the C-terminal half of the ARRDC1). The disclosure also contemplates variants of ARRDC1, such as fragments of ARRDC1 and/or ARRDC1 proteins that have a degree of identity (e.g., 60%, 70%, 80%, 85%, 90%, 95%, 98%, or 99% identity) to an ARRDC1 protein and are capable if interacting with TSG101. Accordingly, an ARRDC1 protein or a variant thereof may be a protein that comprises a PSAP (SEQ ID NO: 2) motif and a PPXY motif and interacts with TSG101. In some embodiments, the ARRDC1 protein is at least 60%, 65%, 70%, 75%, 80%, 85%, 90%, 95%, 98%, or 99% identical to the amino acid sequence of any one of SEQ ID NOs: 3-5, comprises a PSAP (SEQ ID NO: 2) motif and a PPXY motif, and interacts with TSG101. In some embodiments, the ARRDC1 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, or at least 430 identical contiguous amino acids of any one of SEQ ID NOs: 3-5, comprises a PSAP (SEQ ID NO: 2) motif and a PPXY motif, and interacts with TSG101. In some embodiments, the ARRDC1 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 3-5, comprises a PSAP (SEQ ID NO: 2) motif and a PPXY motif, and interacts with TSG101. In some embodiments, the ARRDC1 protein comprises any one of the amino acid sequences set forth in SEQ ID NOs: 3-5. Exemplary, non-limiting ARRDC1 protein sequences are provided herein, and additional, suitable ARRDC1 protein variants according to aspects of this invention are known in the art. It will be appreciated by those of skill in the art that this invention is not limited in this respect. Exemplary ARRDC1 sequences include the following (PSAP (SEQ ID NO: 2) and PPXY motifs are marked):

>gi|22748653|ref|NP_689498.1| arrestin domain-containing protein 1
[Homo sapiens]
(SEQ ID NO: 3)
MGRVQLFEISLSHGRVVYSPGEPLAGTVRVRLGAPLPFRAIRVTCIGSCGVSNKANDT
AWVVEEGYFNSSLSLADKGSLPAGEHSFPFQFLLPATAPTSFEGPFGKIVHQVRAAIH
TPRFSKDHKCSLVFYILSPLNLNSIPDIEQPNVASATKKFSYKLVKTGSVVLTASTDLR
GYVVGQALQLHADVENQSGKDTSPVVASLLQKVSYKAKRWIHDVRTIAEVEGAGV
KAWRRAQWHEQILVPALPQSALPGCSLIHIDYYLQVSLKAPEATVTLPVFIGNIAVNH

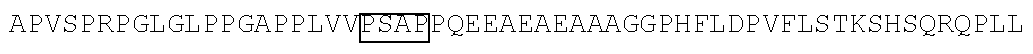

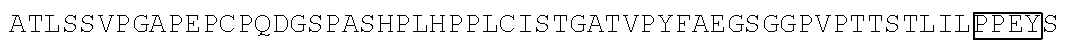

SWGYPYEAPPSYEQSCGGVEPSLTPES
>gi|244798004|ref|NP_001155957.1| arrestin domain-containing protein 1 isoform a
[Mus musculus]
(SEQ ID NO: 4)
MGRVQLFEIRLSQGRVVYGPGEPLAGTVHLRLGAPLPFRAIRVTCMGSCGVSTKAND
GAWVVEESYFNSSLSLADKGSLPAGEHNFPFQFLLPATAPTSFEGPFGKIVHQVRASI
DTPRFSKDHKCSLVFYILSPLNLNSIPDIEQPNVASTTKKFSYKLVKTGNVVLTASTDL
RGYVVGQVLRLQADIENQSGKDTSPVVASLLQKVSYKAKRWIYDVRTIAEVEGTGV
KAWRRAQWQEQILVPALPQSALPGCSLIHIDYYLQVSMKAPEATVTLPLFVGNIAVN

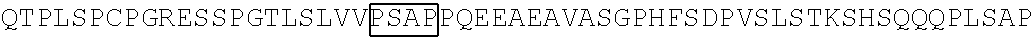

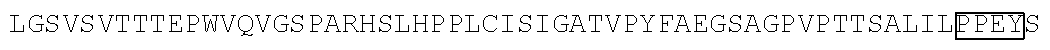

SWGYPYEAPPSYEQSCGAAGTDLGLIPGS
>gi|244798112|ref|NP_848495.2| arrestin domain-containing protein 1 isoform b
[Mus musculus]
(SEQ ID NO: 5)
MGRVQLFEIRLSQGRVVYGPGEPLAGTVHLRLGAPLPFRAIRVTCMGSCGVSTKAND
GAWVVEESYFNSSLSLADKGSLPAGEHNFPFQFLLPATAPTSFEGPFGKIVHQVRASI
DTPRFSKDHKCSLVFYILSPLNLNSIPDIEQPNVASTTKKFSYKLVKTGNVVLTASTDL
RGYVVGQVLRLQADIENQSGKDTSPVVASLLQVSYKAKRWIYDVRTIAEVEGTGVK
AWRRAQWQEQILVPALPQSALPGCSLIHIDYYLQVSMKAPEATVTLPLFVGNIAVNQ

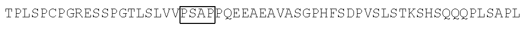

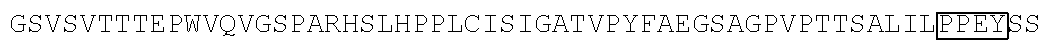

WGYPYEAPPSYEQSCGAAGTDLGLIPGS

### TSG101

In certain embodiments, the inventive microvesicles further comprise TSG101 (tumor susceptibility gene 101). TSG101 belongs to a group of apparently inactive homologs of ubiquitin-conjugating enzymes. The protein contains a coiled-coil domain that interacts with stathmin, a cytosolic phosphoprotein implicated in tumorigenesis. TSG101 is a protein that comprises a UEV domain and interacts with an ARRDC1 protein or a variant thereof. As referred to herein, UEV refers to the Ubiquitin E2 variant domain of approximately 145 amino acids. The structure of the domain contains a α/β fold similar to the canonical E2 enzyme but has an additional N-terminal helix and further lacks the two C-terminal helices. Often found in TSG101/Vps23 proteins, the UEV interacts with a ubiquitin molecule and is essential for the trafficking of a number of ubiquitylated payloads to multivesicular bodies (MVBs). Furthermore, the UEV domain can bind to Pro-Thr/Ser-Ala-Pro peptide ligands, a fact exploited by viruses such as HIV. Thus, the TSG101 UEV domain binds to the PTAP tetrapeptide motif in the viral Gag protein that is involved in viral budding. The disclosure also contemplates variants of TSG101, such as fragments of TSG101 and/or TSG101 proteins that have a degree of identity (e.g., 60%, 70%, 80%, 85%, 90%, 95%, 98%, or 99% identity) to a TSG101 protein and are capable if interacting with ARRDC1. Accordingly, a TSG101 protein may be a protein that comprises a UEV domain and interacts with ARRDC1. In some embodiments, the TSG101 protein is at least 60%, 65%, 70%, 75%, 80%, 85%, 90%, 95%, 98%, or 99% identical to the amino acid sequence of any one of SEQ ID NOs: 45-47, comprises a UEV domain, and interacts with ARRDC1. In some embodiments, the TSG101 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, or at least 390 identical contiguous amino acids of any one of SEQ ID NOS: 45-47, comprises a UEV domain, and interacts with ARRDC1. In some embodiments, the TSG101 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 45-47 and comprises a UEV domain. In some embodiments, the ARRDC1 protein comprises any one of the amino acid sequences set forth in SEQ ID NOs: 3-5. Exemplary, non-limiting TSG101 protein sequences are provided herein, and additional, suitable TSG101 protein sequences, isoforms, and variants are known in the art. It will be appreciated by those of skill in the art that this invention is not limited in this respect. Exemplary TSG101 sequences include the following sequences (the UEV domain in these sequences includes amino acids 1-145 and is underlined in the sequences below):

>gi|5454140|ref|NP_006283.1| tumor susceptibility
gene 101 protein [Homo sapiens]
(SEQ ID NO: 45)
MAVSESQLKKMVSKYKYRDLTVRETVNVITLYKDLKPVLDSYVFNDGSSR
ELMNLTGTIPVPYRGNTYNIPICLWLLDTYPYNPPICFVKPTSSMTIKTG
KHVDANGKIYLPYLHEWKHPQSDLLGLIQVMIVVFGDEPPVFSRPISASY
PPYQATGPPNTSYMPGMPGGISPYPSGYPPNPSGYPGCPYPPGGPYPATT
SSQYPSQPPVTTVGPSRDGTISEDTIRASLISAVSDKLRWRMKEEMDRAQ
AELNALKRTEEDLKKGHQKLEEMVTRLDQEVAEVDKNIELLKKKDEELSS
ALEKMENQSENNDIDEVIIPTAPLYKQILNLYAEENAIEDTIFYLGEALR
RGVIDLDVFLKHVRLLSRKQFQLRALMQKARKTAGLSDLY
>gi|11230780|ref|NP_068684.1| tumor susceptibility
gene 101 protein [Mus musculus]
(SEQ ID NO: 46)
MAVSESQLKKMMSKYKYRDLTVRQTVNVIAMYKDLKPVLDSYVFNDGSSR
ELVNLTGTIPVRYRGNIYNIPICLWLLDTYPYNPPICFVKPTSSMTIKTG
KHVDANGKIYLPYLHDWKHPRSELLELIQIMIVIFGEEPPVFSRPTVSAS
YPPYTATGPPNTSYMPGMPSGISAYPSGYPPNPSGYPGCPYPPAGPYPAT
TSSQYPSQPPVTTVGPSRDGTISEDTIRASLISAVSDKLRWRMKEEMDGA
QAELNALKRTEEDLKKGHQKLEEMVTRLDQEVAEVDKNIELLKKKDEELS
SALEKMENQSENNDIDEVIIPTAPLYKQILNLYAEENAIEDTIFYLGEAL
RRGVIDLDVFLKHVRLLSRKQFQLRALMQKARKTAGLSDLY
>gi|48374087|ref|NP_853659.2| tumor susceptibility
gene 101 protein [Rattus norvegicus]
(SEQ ID NO: 47)
MAVSESQLKKMMSKYKYRDLTVRQTVNVIAMYKDLKPVLDSYVFNDGSSR
ELVNLTGTIPVRYRGNIYNIPICLWLLDTYPYNPPICFVKPTSSMTIKTG
KHVDANGKIYLPYLHDWKHPRSELLELIQIMIVIFGEEPPVFSRPTVSAS
YPPYTAAGPPNTSYLPSMPSGISAYPSGYPPNPSGYPGCPYPPAGPYPAT
TSSQYPSQPPVTTAGPSRDGTISEDTIRASLISAVSDKLRWRMKEEMDGA
QAELNALKRTEEDLKKGHQKLEEMVTRLDQEVAEVDKNIELLKKKDEELS
SALEKMENQSENNDIDEVIIPTAPLYKQILNLYAEENAIEDTIFYLGEAL
RRGVIDLDVFLKHVRLLSRKQFQLRALMQKARKTAGLSDLY

The structure of UEV domains is known to those of skill in the art (see, e.g., Owen Pornillos et al., Structure and functional interactions of the Tsg101 UEV domain, EMBO J. 2002 May 15; 21 (10): 2397-2406, the entire contents of which are incorporated herein by reference).

### Nipah Virus (NiV) Glycoproteins and Fusion Proteins

In some aspects, the present disclosure provides ARMMs modified with NiV glycoproteins and fusion proteins. In some embodiments, the ARMMs disclosed herein comprise a NiV glycoprotein variant fused to an antibody or antigen-binding fragment, wherein the NiV glycoprotein variant does not recognize and/or bind to an ephrin receptor, and a NiV fusion protein. Such modification of ARMMs allows them to be targeted for delivery to a particular cell type, as described herein, and the NiV glycoprotein variant may be fused to various antibodies or antigen-binding fragments that target different cell types.

Viral glycoproteins typically have two important functions: binding and attachment to a target cell, and fusion to the cell membrane. Typically, a single viral glycoprotein is responsible for performing both of these functions. In NiV, however, these functions are split between two different proteins (i.e., the NiV glycoprotein and the NiV fusion protein as described herein). Use of the NiV glycoprotein and fusion protein in the ARMMs described herein therefore allows the NiV glycoprotein to be modified and engineered (e.g., fused to an antibody or antigen-binding fragment to target the ARMMs to particular cell types) without adversely affecting or impacting the function of the fusion protein and the ability of the ARMMs to enter a target cell. The glycoprotein can thus be engineered to eliminate its endogenous binding to ephrin receptors, and a targeting domain (i.e., the antibodies and antigen-binding fragments described herein) can be fused to the glycoprotein. Such a targeting domain can target essentially any protein or other motif on the surface of a target cell.

The wild-type NiV glycoprotein comprises the following amino acid sequence:

(SEQ ID NO: 1)
MPAENKKVRFENTTSDKGKIPSKVIKSYYGTMDIKKINEGLLDSKILSAF
NTVIALLGSIVIIVMNIMIIQNYTRSTDNQAVIKDALQGIQQQIKGLADK
IGTEIGPKVSLIDTSSTITIPANIGLLGSKISQSTASINENVNEKCKFTL
PPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRII
GVGEVLDRGDEVPSLFMTNVWTPPNPNTVYHCSAVYNNEFYYVLCAVSTV
GDPILNSTYWSGSLMMTRLAVKPKSNGGGYNQHQLALRSIEKGRYDKVMP
YGPSGIKQGDTLYFPAVGFLVRTEFKYNDSNCPITKCQYSKPENCRLSMG
IRPNSHYILRSGLLKYNLSDGENPKVVFIEISDQRLSIGSPSKIYDSLGQ
PVFYQASFSWDTMIKFGDVLTVNPLVVNWRNNTVISRPGQSQCPRFNTCP
EICWEGVYNDAFLIDRINWISAGVFLDSNQTAENPVFTVFKDNEILYRAQ
LASEDTNAQKTITNCFLLKNKIWCISLVEIYDTGDNVIRPKLFAVKIPEQ
CT

In some embodiments, the ARMMs described herein comprise a NiV glycoprotein variant that is at least 70%, at least 75%, at least 80%, at least 85%, at least 90%, at least 95%, at least 96%, at least 97%, at least 98%, or at least 99% identical to a wild-type NiV glycoprotein of SEQ ID NO: 1.

The wild-type NiV fusion protein comprises the following amino acid sequence:

(SEQ ID NO: 6)
MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYINTNLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTVNPSLISMLSMIILYVL
SIASLCIGLITFISFIIVEKKRNTYSRLEDRRVRPTSSGDLYYIGT

In some embodiments, the ARMMs described herein comprise a NiV fusion protein variant that is at least 70%, at least 75%, at least 80%, at least 85%, at least 90%, at least 95%, at least 96%, at least 97%, at least 98%, or at least 99% identical to a wild-type NiV fusion protein of SEQ ID NO: 6.

In various embodiments, the NiV glycoprotein variant and NiV fusion protein used in the microvesicles described herein may be modified (e.g., with amino acid substitutions or truncations) relative to the wild-type NiV glycoprotein and fusion protein. For example, the NiV glycoprotein and NiV fusion protein may be modified in any way described in U.S. Pat. App. No. US2019144885A1 and Bender, R. R. et al. Receptor-Targeted Nipah Virus Glycoproteins Improve Cell-Type Selective Gene Delivery and Reveal a Preference for Membrane-Proximal Cell Attachment. PLOS Pathogens 2016, 12 (6): e1005641, both of which are incorporated herein by reference.

In some embodiments, the NiV glycoprotein variant comprises one or more amino acid substitutions relative to a wild-type NiV glycoprotein of SEQ ID NO: 1. Such amino acid substitutions may be introduced, for example, to partially reduce or completely eliminate the ability of the NiV glycoprotein to bind its endogenous target (i.e., ephrin receptors). In some embodiments, the one or more amino acid substitutions are selected from the group consisting of Y389X, E501X, W504X, Q530X, E533X, and I588X, wherein X is any amino acid. In some embodiments, the one or more amino acid substitutions are selected from the group consisting of Y389A, E501A, W504A, Q530A, E533A, and I588A. In certain embodiments, the NiV glycoprotein variant comprises amino acid substitutions E501A, W504A, Q530A, and E533A relative to a wild-type NiV glycoprotein of SEQ ID NO: 1.

In addition to the amino acid substitutions described herein, both the NiV glycoprotein and NiV fusion protein may also be truncated for use in the presently described microvesicles. In some embodiments, the NiV glycoprotein variant further comprises a C-terminal truncation. In some embodiments, the NiV glycoprotein variant further comprises a C-terminal truncation of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, or more than about 50 amino acids in length. In certain embodiments, the NiV glycoprotein variant further comprises a C-terminal truncation of about 33 or about 34 amino acids in length.

In some embodiments, the NiV glycoprotein used in the ARMMs of the present disclosure comprises the amino acid sequence of any one of SEO ID NOs: 63-68:

(SEQ ID NO: 63)
MPAENKKVRFENTTSDKGKIPSKVIKSYYGTMDIKKINEGLLDSKILSAF
NTVIALLGSIVIIVMNIMIIQNYTRSTDNQAVIKDALQGIQQQIKGLADK
IGTEIGPKVSLIDTSSTITIPANIGLLGSKISQSTASINENVNEKCKFTL
PPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRII
GVGEVLDRGDEVPSLFMTNVWTPPNPNTVYHCSAVYNNEFYYVLCAVSTV
GDPILNSTYWSGSLMMTRLAVKPKSNGGGYNQHQLALRSIEKGRYDKVMP
YGPSGIKQGDTLYFPAVGFLVRTEFKYNDSNCPITKCQASKPENCRLSMG
IRPNSHYILRSGLLKYNLSDGENPKVVFIEISDQRLSIGSPSKIYDSLGQ
PVFYQASFSWDTMIKFGDVLTVNPLVVNWRNNTVISRPGQSQCPRFNTCP
AICAEGVYNDAFLIDRINWISAGVFLDSNATAANPVFTVFKDNEILYRAQ
LASEDTNAQKTITNCFLLKNKIWCISLVEIYDTGDNVARPKLFAVKIPEQ
CT
(SEQ ID NO: 64)
MPAENKKVRFENTTSDKGKIPSKVIKSYYGTMDIKKINEGLLDSKILSAF
NTVIALLGSIVIIVMNIMIIQNYTRSTDNQAVIKDALQGIQQQIKGLADK
IGTEIGPKVSLIDTSSTITIPANIGLLGSKISQSTASINENVNEKCKFTL
PPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRII
GVGEVLDRGDEVPSLFMTNVWTPPNPNTVYHCSAVYNNEFYYVLCAVSTV
GDPILNSTYWSGSLMMTRLAVKPKSNGGGYNQHQLALRSIEKGRYDKVMP
YGPSGIKQGDTLYFPAVGFLVRTEFKYNDSNCPITKCQYSKPENCRLSMG
IRPNSHYILRSGLLKYNLSDGENPKVVFIEISDQRLSIGSPSKIYDSLGQ
PVFYQASFSWDTMIKFGDVLTVNPLVVNWRNNTVISRPGQSQCPRFNTCP
AICAEGVYNDAFLIDRINWISAGVFLDSNATAANPVFTVFKDNEILYRAQ
LASEDTNAQKTITNCFLLKNKIWCISLVEIYDTGDNVARPKLFAVKIPEQ
CT
(SEQ ID NO: 65)
MPAENKKVRFENTTSDKGKIPSKVIKSYYGTMDIKKINEGLLDSKILSAF
NTVIALLGSIVIIVMNIMIIQNYTRSTDNQAVIKDALQGIQQQIKGLADK
IGTEIGPKVSLIDTSSTITIPANIGLLGSKISQSTASINENVNEKCKFTL
PPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRII
GVGEVLDRGDEVPSLFMTNVWTPPNPNTVYHCSAVYNNEFYYVLCAVSTV
GDPILNSTYWSGSLMMTRLAVKPKSNGGGYNQHQLALRSIEKGRYDKVMP
YGPSGIKQGDTLYFPAVGFLVRTEFKYNDSNCPITKCQASKPENCRLSMG
IRPNSHYILRSGLLKYNLSDGENPKVVFIEISDQRLSIGSPSKIYDSLGQ
PVFYQASFSWDTMIKFGDVLTVNPLVVNWRNNTVISRPGQSQCPRFNTCP
AICAEGVYNDAFLIDRINWISAGVFLDSNATAANPVFTVFKDNEILYRAQ
LASEDTNAQKTITNCFLLK
(SEQ ID NO: 66)
MPAENKKVRFENTTSDKGKIPSKVIKSYYGTMDIKKINEGLLDSKILSAF
NTVIALLGSIVIIVMNIMIIQNYTRSTDNQAVIKDALQGIQQQIKGLADK
IGTEIGPKVSLIDTSSTITIPANIGLLGSKISQSTASINENVNEKCKFTL
PPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRII
GVGEVLDRGDEVPSLFMTNVWTPPNPNTVYHCSAVYNNEFYYVLCAVSTV
GDPILNSTYWSGSLMMTRLAVKPKSNGGGYNQHQLALRSIEKGRYDKVMP
YGPSGIKQGDTLYFPAVGFLVRTEFKYNDSNCPITKCQYSKPENCRLSMG
IRPNSHYILRSGLLKYNLSDGENPKVVFIEISDQRLSIGSPSKIYDSLGQ
PVFYQASFSWDTMIKFGDVLTVNPLVVNWRNNTVISRPGQSQCPRFNTCP
AICAEGVYNDAFLIDRINWISAGVFLDSNATAANPVFTVFKDNEILYRAQ
LASEDTNAQKTITNCFLLK
(SEQ ID NO: 67)
MPAENKKVRFENTTSDKGKIPSKVIKSYYGTMDIKKINEGLLDSKILSAF
NTVIALLGSIVIIVMNIMIIQNYTRSTDNQAVIKDALQGIQQQIKGLADK
IGTEIGPKVSLIDTSSTITIPANIGLLGSKISQSTASINENVNEKCKFTL
PPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRII
GVGEVLDRGDEVPSLFMTNVWTPPNPNTVYHCSAVYNNEFYYVLCAVSTV
GDPILNSTYWSGSLMMTRLAVKPKSNGGGYNQHQLALRSIEKGRYDKVMP
YGPSGIKQGDTLYFPAVGFLVRTEFKYNDSNCPITKCQASKPENCRLSMG
IRPNSHYILRSGLLKYNLSDGENPKVVFIEISDQRLSIGSPSKIYDSLGQ
PVFYQASFSWDTMIKFGDVLTVNPLVVNWRNNTVISRPGQSQCPRFNTCP
AICAEGVYNDAFLIDRINWISAGVFLDSNATAANPVFTVFKDNEILYRAQ
LASEDTNAQKTITNCFLL
(SEQ ID NO: 68)
MPAENKKVRFENTTSDKGKIPSKVIKSYYGTMDIKKINEGLLDSKILSAF
NTVIALLGSIVIIVMNIMIIQNYTRSTDNQAVIKDALQGIQQQIKGLADK
IGTEIGPKVSLIDTSSTITIPANIGLLGSKISQSTASINENVNEKCKFTL
PPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRII
GVGEVLDRGDEVPSLFMTNVWTPPNPNTVYHCSAVYNNEFYYVLCAVSTV
GDPILNSTYWSGSLMMTRLAVKPKSNGGGYNQHQLALRSIEKGRYDKVMP
YGPSGIKQGDTLYFPAVGFLVRTEFKYNDSNCPITKCQYSKPENCRLSMG
IRPNSHYILRSGLLKYNLSDGENPKVVFIEISDQRLSIGSPSKIYDSLGQ
PVFYQASFSWDTMIKFGDVLTVNPLVVNWRNNTVISRPGQSQCPRFNTCP
AICAEGVYNDAFLIDRINWISAGVFLDSNATAANPVFTVFKDNEILYRAQ
LASEDTNAQKTITNCFLL

In some embodiments, the NiV fusion protein comprises a C-terminal truncation. In some embodiments, the NiV fusion protein comprises a C-terminal truncation of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, or more than about 50 amino acids in length. In certain embodiments, the NiV fusion protein comprises a C-terminal truncation of about 22 amino acids in length.

In some embodiments, the NiV fusion protein used in the ARMMs of the present disclosure comprises the amino acid sequence of SEQ ID NO: 69:

(SEQ ID NO: X7)
MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYINTNLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTVNPSLISMLSMIILYVL
SIASLCIGLITFISFIIVEKKRNT

The NiV glycoprotein variants used in the ARMMs of the present disclosure may also be fused to an antibody or antigen-binding fragment to target the ARMMs to particular cell types as described herein. In some embodiments, the NiV glycoproteins used in the microvesicles described herein are fused to a single-chain variable fragment (scFv). In certain embodiments, the NiV glycoproteins used in the microvesicles described herein are fused to a nanobody. In certain embodiments, the NiV glycoproteins used in the microvesicles described herein are fused to a DARPin.

### Expression Constructs

Some aspects of this invention provide expression constructs for encoding a gene product or gene products that induce or facilitate the generation of ARMMs in cells harboring such a construct. In some embodiments, the expression constructs described herein encode a fusion proteins as described herein, such as ARRDC1 fusion proteins and TSG101 fusion proteins. In some embodiments, the expression constructs encode an ARRDC1 protein, or variant thereof, and/or a TSG101 protein, or variant thereof. In some embodiments, overexpression of either or both of these gene products in a cell increase the production of ARMMs in the cell, thus turning the cell into a microvesicle producing cell. In some embodiments, such an expression construct comprises at least one restriction or recombination site that allows in-frame cloning of a protein sequence to be fused, either at the C-terminus, or at the N-terminus of the encoded ARRDC1, or variant thereof. As another example, an expression construct comprises at least one restriction or recombination site that allows in-frame cloning of a protein sequence to be fused either at the C-terminus, or at the N-terminus of one or more encoded WW domains.

In some embodiments, the expression construct comprises (a) a nucleotide sequence encoding an ARRDC1 protein, or variant thereof, operably linked to a heterologous promoter, and (b) a restriction site or a recombination site positioned adjacent to the ARRDC1-encoding nucleotide sequence allowing for the insertion of a nucleotide sequencing encoding a payload protein, or an RNA binding protein or RNA binding protein variant sequence, in frame with the ARRDC1-encoding nucleotide sequence. In some embodiments, the heterologous promoter may be a constitutive promoter. In some embodiments, the heterologous promoter may be an inducible promoter. Some aspects of this invention provide an expression construct comprising (a) a nucleotide sequence encoding a TSG101 protein, or variant thereof, operably linked to a heterologous promoter, and (b) a restriction site or a recombination site positioned adjacent to the TSG101-encoding nucleotide sequence allowing for the insertion of a nucleotide sequence encoding a protein agent, or an RNA binding protein, DNA binding protein, or variant sequence thereof, in frame with the TSG101-encoding nucleotide sequence. In some embodiments, the heterologous promoter may be a constitutive promoter. In some embodiments, the heterologous promoter may be an inducible promoter.

Some aspects of this invention provide an expression construct comprising (a) a nucleotide sequence encoding a WW domain, or variant thereof, operably linked to a heterologous promoter, and (b) a restriction site or a recombination site positioned adjacent to the WW domain-encoding nucleotide sequence allowing for the insertion of a protein agent or RNA binding protein, or a protein variant sequence thereof in frame with the WW domain-encoding nucleotide sequence. In some embodiments, the heterologous promoter may be a constitutive promoter. In some embodiments, the heterologous promoter may be an inducible promoter. The expression constructs may encode a payload protein, or an RNA binding protein fused to at least one WW domain. In some embodiments, the expression constructs encode a payload protein or an RNA binding protein, or a variant thereof, fused to at least one WW domain, or variant thereof. Any of the expression constructs, described herein, may encode any WW domain or variant thereof. In some embodiments, the heterologous promoter may be a constitutive promoter. In some embodiments, the heterologous promoter may be an inducible promoter.

The expression constructs, described herein, may comprise any nucleic acid sequence capable of encoding a WW domain or variant thereof. For example, a nucleic acid sequence encoding a WW domain or WW domain variant may be from the human ubiquitin ligase WWP1, WWP2, Nedd4-1, Nedd4-2, Smurf1, Smurf2, ITCH, NEDL1, or NEDL2. Exemplary nucleic acid sequences of WW domain-containing proteins are listed below. It should be appreciated that any of the nucleic acids encoding WW domains or WW domain variants of the exemplary proteins may be used in the invention, described herein, and are not meant to be limiting.

Human WWP1 nucleic acid sequence (uniprot.org/uniprot/Q9H0M0):

(SEQ ID NO: 48)
GAATTCGCGGCCGCGTCGACCGCTTCTGTGGCCACGGCAGATGAAACAGA
AAGGCTAAAGAGGGCTGGAGTCAGGGGACTTCTCTTCCACCAGCTTCACG
GTGATGATATGGCATCTGCCAGCTCTAGCCGGGCAGGAGTGGCCCTGCCT
TTTGAGAAGTCTCAGCTCACTTTGAAAGTGGTGTCCGCAAAGCCCAAGGT
GCATAATCGTCAACCTCGAATTAACTCCTACGTGGAGGTGGCGGTGGATG
GACTCCCCAGTGAGACCAAGAAGACTGGGAAGCGCATTGGGAGCTCTGAG
CTTCTCTGGAATGAGATCATCATTTTGAATGTCACGGCACAGAGTCATTT
AGATTTAAAGGTCTGGAGCTGCCATACCTTGAGAAATGAACTGCTAGGCA
CCGCATCTGTCAACCTCTCCAACGTCTTGAAGAACAATGGGGGCAAAATG
GAGAACATGCAGCTGACCCTGAACCTGCAGACGGAGAACAAAGGCAGCGT
TGTCTCAGGCGGAAAACTGACAATTTTCCTGGACGGGCCAACTGTTGATC
TGGGAAATGTGCCTAATGGCAGTGCCCTGACAGATGGATCACAGCTGCCT
TCGAGAGACTCCAGTGGAACAGCAGTAGCTCCAGAGAACCGGCACCAGCC
CCCCAGCACAAACTGCTTTGGTGGAAGATCCCGGACGCACAGACATTCGG
GTGCTTCAGCCAGAACAACCCCAGCAACCGGCGAGCAAAGCCCCGGTGCT
CGGAGCCGGCACCGCCAGCCCGTCAAGAACTCAGGCCACAGTGGCTTGGC
CAATGGCACAGTGAATGATGAACCCACAACAGCCACTGATCCCGAAGAAC
CTTCCGTTGTTGGTGTGACGTCCCCACCTGCTGCACCCTTGAGTGTGACC
CCGAATCCCAACACGACTTCTCTCCCTGCCCCAGCCACACCGGCTGAAGG
AGAGGAACCCAGCACTTCGGGTACACAGCAGCTCCCAGCGGCTGCCCAGG
CCCCCGACGCTCTGCCTGCTGGATGGGAACAGCGAGAGCTGCCCAACGGA
CGTGTCTATTATGTTGACCACAATACCAAGACCACCACCTGGGAGCGGCC
CCTTCCTCCAGGCTGGGAAAAACGCACAGATCCCCGAGGCAGGTTTTACT
ATGTGGATCACAATACTCGGACCACCACCTGGCAGCGTCCGACCGCGGAG
TACGTGCGCAACTATGAGCAGTGGCAGTCGCAGCGGAATCAGCTCCAGGG
GGCCATGCAGCACTTCAGCCAAAGATTCCTATACCAGTTTTGGAGTGCTT
CGACTGACCATGATCCCCTGGGCCCCCTCCCTCCTGGTTGGGAGAAAAGA
CAGGACAATGGACGGGTGTATTACGTGAACCATAACACTCGCACGACCCA
GTGGGAGGATCCCCGGACCCAGGGGATGATCCAGGAACCAGCTTTGCCCC
CAGGATGGGAGATGAAATACACCAGCGAGGGGGTGCGATACTTTGTGGAC
CACAATACCCGCACCACCACCTTTAAGGATCCTCGCCCGGGGTTTGAGTC
GGGGACGAAGCAAGGTTCCCCTGGTGCTTATGACCGCAGTTTTCGGTGGA
AGTATCACCAGTTCCGTTTCCTCTGCCATTCAAATGCCCTACCTAGCCAC
GTGAAGATCAGCGTTTCCAGGCAGACGCTTTTCGAAGATTCCTTCCAACA
GATCATGAACATGAAACCCTATGACCTGCGCCGCCGGCTTTACATCATCA
TGCGTGGCGAGGAGGGCCTGGACTATGGGGGCATCGCCAGAGAGTGGTTT
TTCCTCCTGTCTCACGAGGTGCTCAACCCTATGTATTGTTTATTTGAATA
TGCCGGAAAGAACAATTACTGCCTGCAGATCAACCCCGCCTCCTCCATCA
ACCCGGACCACCTCACCTACTTTCGCTTTATAGGCAGATTCATCGCCATG
GCGCTGTACCATGGAAAGTTCATCGACACGGGCTTCACCCTCCCTTTCTA
CAAGCGGATGCTCAATAAGAGACCAACCCTGAAAGACCTGGAGTCCATTG
ACCCTGAGTTCTACAACTCCATTGTCTGGATCAAAGAGAACAACCTGGAA
GAATGTGGCCTGGAGCTGTACTTCATCCAGGACATGGAGATACTGGGCAA
GGTGACGACCCACGAGCTGAAGGAGGGCGGCGAGAGCATCCGGGTCACGG
AGGAGAACAAGGAAGAGTACATCATGCTGCTGACTGACTGGCGTTTCACC
CGAGGCGTGGAAGAGCAGACCAAAGCCTTCCTGGATGGCTTCAACGAGGT
GGCCCCGCTGGAGTGGCTGCGCTACTTTGACGAGAAAGAGCTGGAGCTGA
TGCTGTGCGGCATGCAGGAGATAGACATGAGCGACTGGCAGAAGAGCACC
ATCTACCGGCACTACACCAAGAACAGCAAGCAGATCCAGTGGTTCTGGCA
GGTGGTGAAGGAGATGGACAACGAGAAGAGGATCCGGCTGCTGCAGTTTG
TCACCGGTACCTGCCGCCTGCCCGTCGGGGGATTTGCCGAACTCATCGGT
AGCAACGGACCACAGAAGTTTTGCATTGACAAAGTTGGCAAGGAAACCTG
GCTGCCCAGAAGCCACACCTGCTTCAACCGTCTGGATCTTCCACCCTACA
AGAGCTACGAACAGCTGAGAGAGAAGCTGCTGTATGCCATTGAGGAGACC
GAGGGCTTTGGACAGGAGTAACCGAGGCCGCCCCTCCCACGCCCCCCAGC
GCACATGTAGTCCTGAGTCCTCCCTGCCTGAGAGGCCACTGGCCCCGCAG
CCCTTGGGAGGCCCCCGTGGATGTGGCCCTGTGTGGGACCACACTGTCAT
CTCGCTGCTGGCAGAAAAGCCTGATCCCAGGAGGCCCTGCAGTTCCCCCG
ACCCGCGGATGGCAGTCTGGAATAAAGCCCCCTAGTTGCCTTTGGCCCCA
CCTTTGCAAAGTTCCAGAGGGCTGACCCTCTCTGCAAAACTCTCCCCTGT
CCTCTAGACCCCACCCTGGGTGTATGTGAGTGTGCAAGGGAAGGTGTTGC
ATCCCCAGGGGCTGCCGCAGAGGCCGGAGACCTCCTGGACTAGTTCGGCG
AGGAGACTGGCCACTGGGGGTGGCTGTTCGGGACTGAGAGCGCCAAGGGT
CTTTGCCAGCAAAGGAGGTTCTGCCTGTAATTGAGCCTCTCTGATGATGG
AGATGAAGTGAAGGTCTGAGGGACGGGCCCTGGGGCTAGGCCATCTCTGC
CTGCCTCCCTAGCAGGCGCCAGCGGTGGAGGCTGAGTCGCAGGACACATG
CCGGCCAGTTAATTCATTCTCAGCAAATGAAGGTTTGTCTAAGCTGCCTG
GGTATCCACGGGACAAAAACAGCAAACTCCCTCCAGACTTTGTCCATGTT
ATAAACTTGAAAGTTGGTTGTTGTTTGTTAGGTTTGCCAGGTTTTTTTGT
TTACGCCTGCTGTCACTTTCCTGTC

Human WWP2 nucleic acid sequence (uniprot.org/uniprot/O00308):

(SEQ ID NO: 49)
GAATTCGCGGCCGCGTCGACCGCTTCTGTGGCCACGGCAGATGAAACAGA
AAGGCTAAAGAGGGCTGGAGTCAGGGGACTTCTCTTCCACCAGCTTCACG
GTGATGATATGGCATCTGCCAGCTCTAGCCGGGCAGGAGTGGCCCTGCCT
TTTGAGAAGTCTCAGCTCACTTTGAAAGTGGTGTCCGCAAAGCCCAAGGT
GCATAATCGTCAACCTCGAATTAACTCCTACGTGGAGGTGGCGGTGGATG
GACTCCCCAGTGAGACCAAGAAGACTGGGAAGCGCATTGGGAGCTCTGAG
CTTCTCTGGAATGAGATCATCATTTTGAATGTCACGGCACAGAGTCATTT
AGATTTAAAGGTCTGGAGCTGCCATACCTTGAGAAATGAACTGCTAGGCA
CCGCATCTGTCAACCTCTCCAACGTCTTGAAGAACAATGGGGGCAAAATG
GAGAACATGCAGCTGACCCTGAACCTGCAGACGGAGAACAAAGGCAGCGT
TGTCTCAGGCGGAAAACTGACAATTTTCCTGGACGGGCCAACTGTTGATC
TGGGAAATGTGCCTAATGGCAGTGCCCTGACAGATGGATCACAGCTGCCT
TCGAGAGACTCCAGTGGAACAGCAGTAGCTCCAGAGAACCGGCACCAGCC
CCCCAGCACAAACTGCTTTGGTGGAAGATCCCGGACGCACAGACATTCGG
GTGCTTCAGCCAGAACAACCCCAGCAACCGGCGAGCAAAGCCCCGGTGCT
CGGAGCCGGCACCGCCAGCCCGTCAAGAACTCAGGCCACAGTGGCTTGGC
CAATGGCACAGTGAATGATGAACCCACAACAGCCACTGATCCCGAAGAAC
CTTCCGTTGTTGGTGTGACGTCCCCACCTGCTGCACCCTTGAGTGTGACC
CCGAATCCCAACACGACTTCTCTCCCTGCCCCAGCCACACCGGCTGAAGG
AGAGGAACCCAGCACTTCGGGTACACAGCAGCTCCCAGCGGCTGCCCAGG
CCCCCGACGCTCTGCCTGCTGGATGGGAACAGCGAGAGCTGCCCAACGGA
CGTGTCTATTATGTTGACCACAATACCAAGACCACCACCTGGGAGCGGCC
CCTTCCTCCAGGCTGGGAAAAACGCACAGATCCCCGAGGCAGGTTTTACT
ATGTGGATCACAATACTCGGACCACCACCTGGCAGCGTCCGACCGCGGAG
TACGTGCGCAACTATGAGCAGTGGCAGTCGCAGCGGAATCAGCTCCAGGG
GGCCATGCAGCACTTCAGCCAAAGATTCCTATACCAGTTTTGGAGTGCTT
CGACTGACCATGATCCCCTGGGCCCCCTCCCTCCTGGTTGGGAGAAAAGA
CAGGACAATGGACGGGTGTATTACGTGAACCATAACACTCGCACGACCCA
GTGGGAGGATCCCCGGACCCAGGGGATGATCCAGGAACCAGCTTTGCCCC
CAGGATGGGAGATGAAATACACCAGCGAGGGGGTGCGATACTTTGTGGAC
CACAATACCCGCACCACCACCTTTAAGGATCCTCGCCCGGGGTTTGAGTC
GGGGACGAAGCAAGGTTCCCCTGGTGCTTATGACCGCAGTTTTCGGTGGA
AGTATCACCAGTTCCGTTTCCTCTGCCATTCAAATGCCCTACCTAGCCAC
GTGAAGATCAGCGTTTCCAGGCAGACGCTTTTCGAAGATTCCTTCCAACA
GATCATGAACATGAAACCCTATGACCTGCGCCGCCGGCTTTACATCATCA
TGCGTGGCGAGGAGGGCCTGGACTATGGGGGCATCGCCAGAGAGTGGTTT
TTCCTCCTGTCTCACGAGGTGCTCAACCCTATGTATTGTTTATTTGAATA
TGCCGGAAAGAACAATTACTGCCTGCAGATCAACCCCGCCTCCTCCATCA
ACCCGGACCACCTCACCTACTTTCGCTTTATAGGCAGATTCATCGCCATG
GCGCTGTACCATGGAAAGTTCATCGACACGGGCTTCACCCTCCCTTTCTA
CAAGCGGATGCTCAATAAGAGACCAACCCTGAAAGACCTGGAGTCCATTG
ACCCTGAGTTCTACAACTCCATTGTCTGGATCAAAGAGAACAACCTGGAA
GAATGTGGCCTGGAGCTGTACTTCATCCAGGACATGGAGATACTGGGCAA
GGTGACGACCCACGAGCTGAAGGAGGGCGGCGAGAGCATCCGGGTCACGG
AGGAGAACAAGGAAGAGTACATCATGCTGCTGACTGACTGGCGTTTCACC
CGAGGCGTGGAAGAGCAGACCAAAGCCTTCCTGGATGGCTTCAACGAGGT
GGCCCCGCTGGAGTGGCTGCGCTACTTTGACGAGAAAGAGCTGGAGCTGA
TGCTGTGCGGCATGCAGGAGATAGACATGAGCGACTGGCAGAAGAGCACC
ATCTACCGGCACTACACCAAGAACAGCAAGCAGATCCAGTGGTTCTGGCA
GGTGGTGAAGGAGATGGACAACGAGAAGAGGATCCGGCTGCTGCAGTTTG
TCACCGGTACCTGCCGCCTGCCCGTCGGGGGATTTGCCGAACTCATCGGT
AGCAACGGACCACAGAAGTTTTGCATTGACAAAGTTGGCAAGGAAACCTG
GCTGCCCAGAAGCCACACCTGCTTCAACCGTCTGGATCTTCCACCCTACA
AGAGCTACGAACAGCTGAGAGAGAAGCTGCTGTATGCCATTGAGGAGACC
GAGGGCTTTGGACAGGAGTAACCGAGGCCGCCCCTCCCACGCCCCCCAGC
GCACATGTAGTCCTGAGTCCTCCCTGCCTGAGAGGCCACTGGCCCCGCAG
CCCTTGGGAGGCCCCCGTGGATGTGGCCCTGTGTGGGACCACACTGTCAT
CTCGCTGCTGGCAGAAAAGCCTGATCCCAGGAGGCCCTGCAGTTCCCCCG
ACCCGCGGATGGCAGTCTGGAATAAAGCCCCCTAGTTGCCTTTGGCCCCA
CCTTTGCAAAGTTCCAGAGGGCTGACCCTCTCTGCAAAACTCTCCCCTGT
CCTCTAGACCCCACCCTGGGTGTATGTGAGTGTGCAAGGGAAGGTGTTGC
ATCCCCAGGGGCTGCCGCAGAGGCCGGAGACCTCCTGGACTAGTTCGGCG
AGGAGACTGGCCACTGGGGGTGGCTGTTCGGGACTGAGAGCGCCAAGGGT
CTTTGCCAGCAAAGGAGGTTCTGCCTGTAATTGAGCCTCTCTGATGATGG
AGATGAAGTGAAGGTCTGAGGGACGGGCCCTGGGGCTAGGCCATCTCTGC
CTGCCTCCCTAGCAGGCGCCAGCGGTGGAGGCTGAGTCGCAGGACACATG
CCGGCCAGTTAATTCATTCTCAGCAAATGAAGGTTTGTCTAAGCTGCCTG
GGTATCCACGGGACAAAAACAGCAAACTCCCTCCAGACTTTGTCCATGTT
ATAAACTTGAAAGTTGGTTGTTGTTTGTTAGGTTTGCCAGGTTTTTTTGT
TTACGCCTGCTGTCACTTTCCTGTC

Human Nedd4-1 nucleic acid sequence (uniprot.org/uniprot/P46934):

(SEQ ID NO: 50)
ACAGTTGCCTGCCCTGGGCGGGGGCGAGCGCGTCCGGTTTGCTGGAAGCG
TTCGGAAATGGCAACTTGCGCGGTGGAGGTGTTCGGGCTCCTGGAGGACG
AGGAAAATTCACGAATTGTGAGAGTAAGAGTTATAGCCGGAATAGGCCTT
GCCAAGAAGGATATATTGGGAGCTAGTGATCCTTACGTGAGAGTGACGTT
ATATGACCCAATGAATGGAGTTCTTACAAGTGTGCAAACAAAAACCATTA
AAAAGAGTTTGAATCCAAAGTGGAATGAAGAAATATTATTCAGAGTTCAT
CCTCAGCAGCACCGGCTTCTTTTTGAAGTGTTTGACGAAAACCGATTGAC
AAGAGATGATTTCCTAGGTCAAGTGGATGTTCCACTTTATCCATTACCGA
CAGAAAATCCAAGATTGGAGAGACCATATACATTTAAGGATTTTGTTCTT
CATCCAAGAAGTCACAAATCAAGAGTTAAAGGTTATCTGAGACTAAAAAT
GACTTATTTACCTAAAACCAGTGGCTCAGAAGATGATAATGCAGAACAGG
CTGAGGAATTAGAGCCTGGCTGGGTTGTTTTGGACCAACCAGATGCTGCT
TGCCATTTGCAGCAACAACAAGAACCTTCTCCTCTACCTCCAGGGTGGGA
AGAGAGGCAGGATATCCTTGGAAGGACCTATTATGTAAACCATGAATCTA
GAAGAACACAGTGGAAAAGACCAACCCCTCAGGACAACCTAACAGATGCT
GAGAATGGCAACATTCAACTGCAAGCACAACGTGCATTTACCACCAGGCG
GCAGATATCCGAGGAAACAGAAAGTGTTGACAACCAAGAGTCTTCCGAGA
ACTGGGAAATTATAAGAGAAGATGAAGCCACCATGTATAGCAGCCAGGCC
TTCCCATCACCTCCACCGTCAAGTAACTTGGATGTTCCAACTCATCTTGC
AGAAGAATTGAATGCCAGACTCACCATTTTTGGAAATTCAGCCGTGAGCC
AGCCAGCATCGAGCTCAAATCATTCCAGCAGAAGAGGCAGCTTACAAGCC
TATACTTTTGAGGAACAACCTACACTTCCTGTGCTTTTGCCTACTTCATC
TGGATTACCACCAGGTTGGGAAGAAAAACAAGATGAAAGAGGAAGATCAT
ATTATGTAGATCACAATTCCAGAACGACTACTTGGACAAAGCCCACTGTA
CAGGCCACAGTGGAGACCAGTCAGCTGACCTCAAGCCAGAGTTCTGCAGG
CCCTCAATCACAAGCCTCCACCAGTGATTCAGGCCAGCAGGTGACCCAGC
CATCTGAAATTGAGCAAGGATTCCTTCCTAAAGGCTGGGAAGTCCGGCAT
GCACCAAATGGGAGGCCTTTCTTTATTGACCACAACACTAAAACCACCAC
CTGGGAAGATCCAAGATTGAAAATTCCAGCCCATCTGAGAGGAAAGACAT
CACTTGATACTTCCAATGATCTAGGGCCTTTACCTCCAGGATGGGAAGAG
AGAACTCACACAGATGGAAGAATCTTCTACATAAATCACAATATAAAAAG
AACACAATGGGAAGATCCTCGGTTGGAGAATGTAGCAATAACTGGACCAG
CAGTGCCCTACTCCAGGGATTACAAAAGAAAGTATGAGTTCTTCCGAAGA
AAGTTGAAGAAGCAGAATGACATTCCAAACAAATTTGAAATGAAACTTCG
CCGAGCAACTGTTCTTGAAGACTCTTACCGGAGAATTATGGGTGTCAAGA
GAGCAGACTTCCTGAAGGCTCGACTGTGGATTGAGTTTGATGGTGAAAAG
GGATTGGATTATGGAGGAGTTGCCAGAGAATGGTTCTTCCTGATCTCAAA
GGAAATGTTTAACCCTTATTATGGGTTGTTTGAATATTCTGCTACGGACA
ATTATACCCTACAGATAAATCCAAACTCTGGATTGTGTAACGAAGATCAC
CTCTCTTACTTCAAGTTTATTGGTCGGGTAGCTGGAATGGCAGTTTATCA
TGGCAAACTGTTGGATGGTTTTTTCATCCGCCCATTTTACAAGATGATGC
TTCACAAACCAATAACCCTTCATGATATGGAATCTGTGGATAGTGAATAT
TACAATTCCCTAAGATGGATTCTTGAAAATGACCCAACAGAATTGGACCT
CAGGTTTATCATAGATGAAGAACTTTTTGGACAGACACATCAACATGAGC
TGAAAAATGGTGGATCAGAAATAGTTGTCACCAATAAGAACAAAAAGGAA
TATATTTATCTTGTAATACAATGGCGATTTGTAAACCGAATCCAGAAGCA
AATGGCTGCTTTTAAAGAGGGATTCTTTGAACTAATACCACAGGATCTCA
TCAAAATTTTTGATGAAAATGAACTAGAGCTTCTTATGTGTGGACCGGGA
GATGTTGATGTGAATGACTGGAGGGAACATACAAAGTATAAAAATGGCTA
CAGTGCAAATCATCAGGTTATACAGTGGTTTTGGAAGGCTGTTTTAATGA
TGGATTCAGAAAAAAGAATAAGATTACTTCAGTTTGTCACTGGCACATCT
CGGGTGCCTATGAATGGATTTGCTGAACTATACGGTTCAAATGGACCACA
GTCATTTACAGTTGAACAGTGGGGTACTCCTGAAAAGCTGCCAAGAGCTC
ATACCTGTTTTAATCGCCTGGACTTGCCACCTTATGAATCATTTGAAGAA
TTATGGGATAAACTTCAGATGGCAATTGAAAACACCCAGGGCTTTGATGG
AGTTGATTAGATTACAAATAACAATCTGTAGTGTTTTTACTGCCATAGTT
TTATAACCAAAATCTTGACTTAAAATTTTCCGGGGAACTACTAAAATGTG
GCCACTGAGTCTTCCCAGATCTTGAAGAAAATCATATAAAAAGCATTTGA
AGAAATAGTACGAC

Human Nedd4-2 nucleic acid sequence (>gi|345478679|ref|NM_015277.5|Homo sapiens neural precursor cell expressed, developmentally down-regulated 4-like, E3 ubiquitin protein ligase (NEDD4L), transcript variant d, mRNA):

(SEQ ID NO: 51)
ATGGCGACCGGGCTCGGGGAGCCGGTCTATGGACTTTCCGAAGACGAGGG
AGAGTCCCGTATTCTCAGAGTAAAAGTTGTTTCTGGAATTGATCTCGCCA
AAAAGGACATCTTTGGAGCCAGTGATCCGTATGTGAAACTTTCATTGTAC
GTAGCGGATGAGAATAGAGAACTTGCTTTGGTCCAGACAAAAACAATTAA
AAAGACACTGAACCCAAAATGGAATGAAGAATTTTATTTCAGGGTAAACC
CATCTAATCACAGACTCCTATTTGAAGTATTTGACGAAAATAGACTGACA
CGAGACGACTTCCTGGGCCAGGTGGACGTGCCCCTTAGTCACCTTCCGAC
AGAAGATCCAACCATGGAGCGACCCTATACATTTAAGGACTTTCTCCTCA
GACCAAGAAGTCATAAGTCTCGAGTTAAGGGATTTTTGCGATTGAAAATG
GCCTATATGCCAAAAAATGGAGGTCAAGATGAAGAAAACAGTGACCAGAG
GGATGACATGGAGCATGGATGGGAAGTTGTTGACTCAAATGACTCGGCTT
CTCAGCACCAAGAGGAACTTCCTCCTCCTCCTCTGCCTCCCGGGTGGGAA
GAAAAAGTGGACAATTTAGGCCGAACTTACTATGTCAACCACAACAACCG
GACCACTCAGTGGCACAGACCAAGCCTGATGGACGTGTCCTCGGAGTCGG
ACAATAACATCAGACAGATCAACCAGGAGGCAGCACACCGGCGCTTCCGC
TCCCGCAGGCACATCAGCGAAGACTTGGAGCCCGAGCCCTCGGAGGGCGG
GGATGTCCCCGAGCCTTGGGAGACCATTTCAGAGGAAGTGAATATCGCTG
GAGACTCTCTCGGTCTGGCTCTGCCCCCACCACCGGCCTCCCCAGGATCT
CGGACCAGCCCTCAGGAGCTGTCAGAGGAACTAAGCAGAAGGCTTCAGAT
CACTCCAGACTCCAATGGGGAACAGTTCAGCTCTTTGATTCAAAGAGAAC
CCTCCTCAAGGTTGAGGTCATGCAGTGTCACCGACGCAGTTGCAGAACAG
GGCCATCTACCACCGCCATCAGTGGCCTATGTACATACCACGCCGGGTCT
GCCTTCAGGCTGGGAAGAAAGAAAAGATGCTAAGGGGCGCACATACTATG
TCAATCATAACAATCGAACCACAACTTGGACTCGACCTATCATGCAGCTT
GCAGAAGATGGTGCGTCCGGATCAGCCACAAACAGTAACAACCATCTAAT
CGAGCCTCAGATCCGCCGGCCTCGTAGCCTCAGCTCGCCAACAGTAACTT
TATCTGCCCCGCTGGAGGGTGCCAAGGACTCACCCGTACGTCGGGCTGTG
AAAGACACCCTTTCCAACCCACAGTCCCCACAGCCATCACCTTACAACTC
CCCCAAACCACAACACAAAGTCACACAGAGCTTCTTGCCACCCGGCTGGG
AAATGAGGATAGCGCCAAACGGCCGGCCCTTCTTCATTGATCATAACACA
AAGACTACAACCTGGGAAGATCCACGTTTGAAATTTCCAGTACATATGCG
GTCAAAGACATCTTTAAACCCCAATGACCTTGGCCCCCTTCCTCCTGGCT
GGGAAGAAAGAATTCACTTGGATGGCCGAACGTTTTATATTGATCATAAT
AGCAAAATTACTCAGTGGGAAGACCCAAGACTGCAGAACCCAGCTATTAC
TGGTCCGGCTGTCCCTTACTCCAGAGAATTTAAGCAGAAATATGACTACT
TCAGGAAGAAATTAAAGAAACCTGCTGATATCCCCAATAGGTTTGAAATG
AAACTTCACAGAAATAACATATTTGAAGAGTCCTATCGGAGAATTATGTC
CGTGAAAAGACCAGATGTCCTAAAAGCTAGACTGTGGATTGAGTTTGAAT
CAGAGAAAGGTCTTGACTATGGGGGTGTGGCCAGAGAATGGTTCTTCTTA
CTGTCCAAAGAGATGTTCAACCCCTACTACGGCCTCTTTGAGTACTCTGC
CACGGACAACTACACCCTTCAGATCAACCCTAATTCAGGCCTCTGTAATG
AGGATCATTTGTCCTACTTCACTTTTATTGGAAGAGTTGCTGGTCTGGCC
GTATTTCATGGGAAGCCTTCATTAGACCATCTTAGATGGTTTTTTTACAA
GATGATGTTGGGAAAGCAGATAACCCTGAATGACATGGAATCTGTGGATA
GTGAATATTACAACTCTTTGAAATGGATCCTGGAGAATGACCCTACTGAG
CTGGACCTCATGTTCTGCATAGACGAAGAAAACTTTGGACAGACATATCA
AGTGGATTTGAAGCCCAATGGGTCAGAAATAATGGTCACAAATGAAAACA
AAAGGGAATATATCGACTTAGTCATCCAGTGGAGATTTGTGAACAGGGTC
CAGAAGCAGATGAACGCCTTCTTGGAGGGATTCACAGAACTACTTCCTAT
TGATTTGATTAAAATTTTTGATGAAAATGAGCTGGAGTTGCTCATGTGCG
GCCTCGGTGATGTGGATGTGAATGACTGGAGACAGCATTCTATTTACAAG
AACGGCTACTGCCCAAACCACCCCGTCATTCAGTGGTTCTGGAAGGCTGT
GCTACTCATGGACGCCGAAAAGCGTATCCGGTTACTGCAGTTTGTCACAG
GGACATCGCGAGTACCTATGAATGGATTTGCCGAACTTTATGGTTCCAAT
GGTCCTCAGCTGTTTACAATAGAGCAATGGGGCAGTCCTGAGAAACTGCC
CAGAGCTCACACATGCTTTAATCGCCTTGACTTACCTCCATATGAAACCT
TTGAAGATTTACGAGAGAAACTTCTCATGGCCGTGGAAAATGCTCAAGGA
TTTGAAGGGGTGGATTAA

Human Smurf1 nucleic acid sequence (uniprot.org/uniprot/Q9HCE7):

(SEQ ID NO: 52)
ATGTCGAACCCCGGGACACGCAGGAACGGCTCCAGCATCAAGATCCGTCT
GACAGTGTTATGTGCCAAGAACCTTGCAAAGAAAGACTTCTTCAGGCTCC
CTGACCCTTTTGCAAAGATTGTCGTGGATGGGTCTGGGCAGTGCCACTCA
ACCGACACTGTGAAAAACACATTGGACCCAAAGTGGAACCAGCACTATGA
TCTATATGTTGGGAAAACGGATTCGATAACCATTAGCGTGTGGAACCATA
AGAAAATTCACAAGAAACAGGGAGCTGGCTTCCTGGGCTGTGTGCGGCTG
CTCTCCAATGCCATCAGCAGATTAAAAGATACCGGATACCAGCGTTTGGA
TCTATGCAAACTAAACCCCTCAGATACTGATGCAGTTCGTGGCCAGATAG
TGGTCAGTTTACAGACACGAGACAGAATAGGAACCGGCGGCTCGGTGGTG
GACTGCAGAGGACTGTTAGAAAATGAAGGAACGGTGTATGAAGACTCCGG
GCCTGGGAGGCCGCTCAGCTGCTTCATGGAGGAACCAGCCCCTTACACAG
ATAGCACCGGTGCTGCTGCTGGAGGAGGGAATTGCAGGTTCGTGGAGTCC
CCAAGTCAAGATCAAAGACTTCAGGCACAGCGGCTTCGAAACCCTGATGT
GCGAGGTTCACTACAGACGCCCCAGAACCGACCACACGGCCACCAGTCCC
CGGAACTGCCCGAAGGCTACGAACAAAGAACAACAGTCCAGGGCCAAGTT
TACTTTTTGCATACACAGACTGGAGTTAGCACGTGGCACGACCCCAGGAT
ACCAAGTCCCTCGGGGACCATTCCTGGGGGAGATGCAGCTTTTCTATACG
AATTCCTTCTACAAGGCCATACATCTGAGCCCAGAGACCTTAACAGTGTG
AACTGTGATGAACTTGGACCACTGCCGCCAGGCTGGGAAGTCAGAAGTAC
AGTTTCTGGGAGGATATATTTTGTAGATCATAATAACCGAACAACCCAGT
TTACAGACCCAAGGTTACACCACATCATGAATCACCAGTGCCAACTCAAG
GAGCCCAGCCAGCCGCTGCCACTGCCCAGTGAGGGCTCTCTGGAGGACGA
GGAGCTTCCTGCCCAGAGATACGAAAGAGATCTAGTCCAGAAGCTGAAAG
TCCTCAGACACGAACTGTCGCTTCAGCAGCCCCAAGCTGGTCATTGCCGC
ATCGAAGTGTCCAGAGAAGAAATCTTTGAGGAGTCTTACCGCCAGATAAT
GAAGATGCGACCGAAAGACTTGAAAAAACGGCTGATGGTGAAATTCCGTG
GGGAAGAAGGTTTGGATTACGGTGGTGTGGCCAGGGAGTGGCTTTACTTG
CTGTGCCATGAAATGCTGAATCCTTATTACGGGCTCTTCCAGTATTCTAC
GGACAATATTTACATGTTGCAAATAAATCCGGATTCTTCAATCAACCCCG
ACCACTTGTCTTATTTCCACTTTGTGGGGCGGATCATGGGGCTGGCTGTG
TTCCATGGACACTACATCAACGGGGGCTTCACAGTGCCCTTCTACAAGCA
GCTGCTGGGGAAGCCCATCCAGCTCTCAGATCTGGAATCTGTGGACCCAG
AGCTGCATAAGAGCTTGGTGTGGATCCTAGAGAACGACATCACGCCTGTA
CTGGACCACACCTTCTGCGTGGAACACAACGCCTTCGGGCGGATCCTGCA
GCATGAACTGAAACCCAATGGCAGAAATGTGCCAGTCACAGAGGAGAATA
AGAAAGAATACGTCCGGTTGTATGTAAACTGGAGGTTTATGAGAGGAATC
GAAGCCCAGTTCTTAGCTCTGCAGAAGGGGTTCAATGAGCTCATCCCTCA
ACATCTGCTGAAGCCTTTTGACCAGAAGGAACTGGAGCTGATCATAGGCG
GCCTGGATAAAATAGACTTGAACGACTGGAAGTCGAACACGCGGCTGAAG
CACTGTGTGGCCGACAGCAACATCGTGCGGTGGTTCTGGCAAGCGGTGGA
GACGTTCGATGAAGAAAGGAGGGCCAGGCTCCTGCAGTTTGTGACTGGGT
CCACGCGAGTCCCGCTCCAAGGCTTCAAGGCTTTGCAAGGTTCTACAGGC
GCGGCAGGGCCCCGGCTGTTCACCATCCACCTGATAGACGCGAACACAGA
CAACCTTCCGAAGGCCCATACCTGCTTTAACCGGATCGACATTCCACCAT
ATGAGTCCTATGAGAAGCTCTACGAGAAGCTGCTGACAGCCGTGGAGGAG
ACCTGCGGGTTTGCTGTGGAGTGA.

Human Smurf2 nucleic acid sequence (uniprot.org/uniprot/Q9HAU4):

(SEQ ID NO: 53)
ATGTCTAACCCCGGACGCCGGAGGAACGGGCCCGTCAAGCTGCGCCTGAC
AGTACTCTGTGCAAAAAACCTGGTGAAAAAGGATTTTTTCCGACTTCCTG
ATCCATTTGCTAAGGTGGTGGTTGATGGATCTGGGCAATGCCATTCTACA
GATACTGTGAAGAATACGCTTGATCCAAAGTGGAATCAGCATTATGACCT
GTATATTGGAAAGTCTGATTCAGTTACGATCAGTGTATGGAATCACAAGA
AGATCCATAAGAAACAAGGTGCTGGATTTCTCGGTTGTGTTCGTCTTCTT
TCCAATGCCATCAACCGCCTCAAAGACACTGGTTATCAGAGGTTGGATTT
ATGCAAACTCGGGCCAAATGACAATGATACAGTTAGAGGACAGATAGTAG
TAAGTCTTCAGTCCAGAGACCGAATAGGCACAGGAGGACAAGTTGTGGAC
TGCAGTCGTTTATTTGATAACGATTTACCAGACGGCTGGGAAGAAAGGAG
AACCGCCTCTGGAAGAATCCAGTATCTAAACCATATAACAAGAACTACGC
AATGGGAGCGCCCAACACGACCGGCATCCGAATATTCTAGCCCTGGCAGA
CCTCTTAGCTGCTTTGTTGATGAGAACACTCCAATTAGTGGAACAAATGG
TGCAACATGTGGACAGTCTTCAGATCCCAGGCTGGCAGAGAGGAGAGTCA
GGTCACAACGACATAGAAATTACATGAGCAGAACACATTTACATACTCCT
CCAGACCTACCAGAAGGCTATGAACAGAGGACAACGCAACAAGGCCAGGT
GTATTTCTTACATACACAGACTGGTGTGAGCACATGGCATGATCCAAGAG
TGCCCAGGGATCTTAGCAACATCAATTGTGAAGAGCTTGGTCCATTGCCT
CCTGGATGGGAGATCCGTAATACGGCAACAGGCAGAGTTTATTTCGTTGA
CCATAACAACAGAACAACACAATTTACAGATCCTCGGCTGTCTGCTAACT
TGCATTTAGTTTTAAATCGGCAGAACCAATTGAAAGACCAACAGCAACAG
CAAGTGGTATCGTTATGTCCTGATGACACAGAATGCCTGACAGTCCCAAG
GTACAAGCGAGACCTGGTTCAGAAACTAAAAATTTTGCGGCAAGAACTTT
CCCAACAACAGCCTCAGGCAGGTCATTGCCGCATTGAGGTTTCCAGGGAA
GAGATTTTTGAGGAATCATATCGACAGGTCATGAAAATGAGACCAAAAGA
TCTCTGGAAGCGATTAATGATAAAATTTCGTGGAGAAGAAGGCCTTGACT
ATGGAGGCGTTGCCAGGGAATGGTTGTATCTCTTGTCACATGAAATGTTG
AATCCATACTATGGCCTCTTCCAGTATTCAAGAGATGATATTTATACATT
GCAGATCAATCCTGATTCTGCAGTTAATCCGGAACATTTATCCTATTTCC
ACTTTGTTGGACGAATAATGGGAATGGCTGTGTTTCATGGACATTATATT
GATGGTGGTTTCACATTGCCTTTTTATAAGCAATTGCTTGGGAAGTCAAT
TACCTTGGATGACATGGAGTTAGTAGATCCGGATCTTCACAACAGTTTAG
TGTGGATACTTGAGAATGATATTACAGGTGTTTTGGACCATACCTTCTGT
GTTGAACATAATGCATATGGTGAAATTATTCAGCATGAACTTAAACCAAA
TGGCAAAAGTATCCCTGTTAATGAAGAAAATAAAAAAGAATATGTCAGGC
TCTATGTGAACTGGAGATTTTTACGAGGCATTGAGGCTCAATTCTTGGCT
CTGCAGAAAGGATTTAATGAAGTAATTCCACAACATCTGCTGAAGACATT
TGATGAGAAGGAGTTAGAGCTCATTATTTGTGGACTTGGAAAGATAGATG
TTAATGACTGGAAGGTAAACACCCGGTTAAAACACTGTACACCAGACAGC
AACATTGTCAAATGGTTCTGGAAAGCTGTGGAGTTTTTTGATGAAGAGCG
ACGAGCAAGATTGCTTCAGTTTGTGACAGGATCCTCTCGAGTGCCTCTGC
AGGGCTTCAAAGCATTGCAAGGTGCTGCAGGCCCGAGACTCTTTACCATA
CACCAGATTGATGCCTGCACTAACAACCTGCCGAAAGCCCACACTTGCTT
CAATCGAATAGACATTCCACCCTATGAAAGCTATGAAAAGCTATATGAAA
AGCTGCTAACAGCCATTGAAGAAACATGTGGATTTGCTGTGGAATGA

Human ITCH nucleic acid sequence (uniprot.org/uniprot/Q96J02):

(SEQ ID NO: 54)
GGAGTCGCCGCCGCCCCGAGTTCCGGTACCATGCATTTCACGGTGGCCTT
GTGGAGACAACGCCTTAACCCAAGGAAGTGACTCAAACTGTGAGAACTCC
AGGTTTTCCAACCTATTGGTGGTATGTCTGACAGTGGATCACAACTTGGT
TCAATGGGTAGCCTCACCATGAAATCACAGCTTCAGATCACTGTCATCTC
AGCAAAACTTAAGGAAAATAAGAAGAATTGGTTTGGACCAAGTCCTTACG
TAGAGGTCACAGTAGATGGACAGTCAAAGAAGACAGAAAAATGCAACAAC
ACAAACAGTCCCAAGTGGAAGCAACCCCTTACAGTTATCGTTACCCCTGT
GAGTAAATTACATTTTCGTGTGTGGAGTCACCAGACACTGAAATCTGATG
TTTTGTTGGGAACTGCTGCATTAGATATTTATGAAACATTAAAGTCAAAC
AATATGAAACTTGAAGAAGTAGTTGTGACTTTGCAGCTTGGAGGTGACAA
AGAGCCAACAGAGACAATAGGAGACTTGTCAATTTGTCTTGATGGGCTAC
AGTTAGAGTCTGAAGTTGTTACCAATGGTGAAACTACATGTTCAGAAAGT
GCTTCTCAGAATGATGATGGCTCCAGATCCAAGGATGAAACAAGAGTGAG
CACAAATGGATCAGATGACCCTGAAGATGCAGGAGCTGGTGAAAATAGGA
GAGTCAGTGGGAATAATTCTCCATCACTCTCAAATGGTGGTTTTAAACCT
TCTAGACCTCCAAGACCTTCACGACCACCACCACCCACCCCACGTAGACC
AGCATCTGTCAATGGTTCACCATCTGCCACTTCTGAAAGTGATGGGTCTA
GTACAGGCTCTCTGCCGCCGACAAATACAAATACAAATACATCTGAAGGA
GCAACATCTGGATTAATAATTCCTCTTACTATATCTGGAGGCTCAGGCCC
TAGGCCATTAAATCCTGTAACTCAAGCTCCCTTGCCACCTGGTTGGGAGC
AGAGAGTGGACCAGCACGGGCGAGTTTACTATGTAGATCATGTTGAGAAA
AGAACAACATGGGATAGACCAGAACCTCTACCTCCTGGCTGGGAACGGCG
GGTTGACAACATGGGACGTATTTATTATGTTGACCATTTCACAAGAACAA
CAACGTGGCAGAGGCCAACACTGGAATCCGTCCGGAACTATGAACAATGG
CAGCTACAGCGTAGTCAGCTTCAAGGAGCAATGCAGCAGTTTAACCAGAG
ATTCATTTATGGGAATCAAGATTTATTTGCTACATCACAAAGTAAAGAAT
TTGATCCTCTTGGTCCATTGCCACCTGGATGGGAGAAGAGAACAGACAGC
AATGGCAGAGTATATTTCGTCAACCACAACACACGAATTACACAATGGGA
AGACCCCAGAAGTCAAGGTCAATTAAATGAAAAGCCCTTACCTGAAGGTT
GGGAAATGAGATTCACAGTGGATGGAATTCCATATTTTGTGGACCACAAT
AGAAGAACTACCACCTATATAGATCCCCGCACAGGAAAATCTGCCCTAGA
CAATGGACCTCAGATAGCCTATGTTCGGGACTTCAAAGCAAAGGTTCAGT
ATTTCCGGTTCTGGTGTCAGCAACTGGCCATGCCACAGCACATAAAGATT
ACAGTGACAAGAAAAACATTGTTTGAGGATTCCTTTCAACAGATAATGAG
CTTCAGTCCCCAAGATCTGCGAAGACGTTTGTGGGTGATTTTTCCAGGAG
AAGAAGGTTTAGATTATGGAGGTGTAGCAAGAGAATGGTTCTTTCTTTTG
TCACATGAAGTGTTGAACCCAATGTATTGCCTGTTTGAATATGCAGGGAA
GGATAACTACTGCTTGCAGATAAACCCCGCTTCTTACATCAATCCAGATC
ACCTGAAATATTTTCGTTTTATTGGCAGATTTATTGCCATGGCTCTGTTC
CATGGGAAATTCATAGACACGGGTTTTTCTTTACCATTCTATAAGCGTAT
CTTGAACAAACCAGTTGGACTCAAGGATTTAGAATCTATTGATCCAGAAT
TTTACAATTCTCTCATCTGGGTTAAGGAAAACAATATTGAGGAATGTGAT
TTGGAAATGTACTTCTCCGTTGACAAAGAAATTCTAGGTGAAATTAAGAG
TCATGATCTGAAACCTAATGGTGGCAATATTCTTGTAACAGAAGAAAATA
AAGAGGAATACATCAGAATGGTAGCTGAGTGGAGGTTGTCTCGAGGTGTT
GAAGAACAGACACAAGCTTTCTTTGAAGGCTTTAATGAAATTCTTCCCCA
GCAATATTTGCAATACTTTGATGCAAAGGAATTAGAGGTCCTTTTATGTG
GAATGCAAGAGATTGATTTGAATGACTGGCAAAGACATGCCATCTACCGT
CATTATGCAAGGACCAGCAAACAAATCATGTGGTTTTGGCAGTTTGTTAA
AGAAATTGATAATGAGAAGAGAATGAGACTTCTGCAGTTTGTTACTGGAA
CCTGCCGATTGCCAGTAGGAGGATTTGCTGATCTCATGGGGAGCAATGGA
CCACAGAAATTCTGCATTGAAAAAGTTGGGAAAGAAAATTGGCTACCCAG
AAGTCATACCTGTTTTAATCGCCTGGACCTGCCACCATACAAGAGCTATG
AGCAACTGAAGGAAAAGCTGTTGTTTGCCATAGAAGAAACAGAAGGATTT
GGACAAGAGTAACTTCTGAGAACTTGCACCATGAATGGGCAAGAACTTAT
TTGCAATGTTTGTCCTTCTCTGCCTGTTGCACATCTTGTAAAATTGGACA
ATGGCTCTTTAGAGAGTTATCTGAGTGTAAGTAAATTAATGTTCTCATTT
AAAAAAAAAAAAAAAAAAA

Human NEDL1 nucleic acid sequence (uniprot.org/uniprot/Q76N89):

(SEQ ID NO: 55)
GCGCATCAGGCGCTGTTGTTGGAGCCGGAACACCGTGCGACTCTGACCGA
ACCGGCCCCCTCCTCGCGCACACACTCGCCGAGCCGCGCGCGCCCCTCCG
CCGTGACAGTGGCCGTGGCCTCCGCTCTCTCGGGGCACCCGGCAGCCAGA
GCGCAGCGAGAGCGGGCGGTCGCCAGGGTCCCCTCCCCAGCCAGTCCCAG
GCGCCCGGTGCACTATGCGGGGCACGTGCGCCCCCCAGCTCTAATCTGCG
CGCTGACAGGAGCATGATCTGTGCCCAGGCCAGGGCTGCCAAGGAATTGA
TGCGCGTACACGTGGTGGGTCATTATGCTGCTACACCTGTGTAGTGTGAA
GAATCTGTACCAGAACAGGTTTTTAGGCCTGGCCGCCATGGCGTCTCCTT
CTAGAAACTCCCAGAGCCGACGCCGGTGCAAGGAGCCGCTCCGATACAGC
TACAACCCCGACCAGTTCCACAACATGGACCTCAGGGGCGGCCCCCACGA
TGGCGTCACCATTCCCCGCTCCACCAGCGACACTGACCTGGTCACCTCGG
ACAGCCGCTCCACGCTCATGGTCAGCAGCTCCTACTATTCCATCGGGCAC
TCTCAGGACCTGGTCATCCACTGGGACATAAAGGAGGAAGTGGACGCTGG
GGACTGGATTGGCATGTACCTCATTGATGAGGTCTTGTCCGAAAACTTTC
TGGACTATAAAAACCGTGGAGTCAATGGTTCTCATCGGGGCCAGATCATC
TGGAAGATCGATGCCAGCTCGTACTTTGTGGAACCTGAAACTAAGATCTG
CTTCAAATACTACCATGGAGTGAGTGGGGCCCTGCGAGCAACCACCCCCA
GTGTCACGGTCAAAAACTCGGCAGCTCCTATTTTTAAAAGCATTGGTGCT
GATGAGACCGTCCAAGGACAAGGAAGTCGGAGGCTGATCAGCTTCTCTCT
CTCAGATTTCCAAGCCATGGGGTTGAAGAAAGGGATGTTTTTCAACCCAG
ACCCTTATCTGAAGATTTCCATTCAGCCTGGGAAACACAGCATCTTCCCC
GCCCTCCCTCACCATGGACAGGAGAGGAGATCCAAGATCATAGGCAACAC
CGTGAACCCCATCTGGCAGGCCGAGCAATTCAGTTTTGTGTCCTTGCCCA
CTGACGTGCTGGAAATTGAGGTGAAGGACAAGTTTGCCAAGAGCCGCCCC
ATCATCAAGCGCTTCTTGGGAAAGCTGTCGATGCCCGTTCAAAGACTCCT
GGAGAGACACGCCATAGGGGATAGGGTGGTCAGCTACACACTTGGCCGCA
GGCTTCCAACAGATCATGTGAGTGGACAGCTGCAATTCCGATTTGAGATC
ACTTCCTCCATCCACCCAGATGATGAGGAGATTTCCCTGAGTACCGAGCC
TGAGTCAGCCCAAATTCAGGACAGCCCCATGAACAACCTGATGGAAAGCG
GCAGTGGGGAACCTCGGTCTGAGGCACCAGAGTCCTCTGAGAGCTGGAAG
CCAGAGCAGCTGGGTGAGGGCAGTGTCCCCGATGGTCCAGGGAACCAAAG
CATAGAGCTTTCCAGACCAGCTGAGGAAGCAGCAGTCATCACGGAGGCAG
GAGACCAGGGCATGGTCTCTGTGGGACCTGAAGGGGCTGGGGAGCTCCTG
GCCCAGGTGCAAAAGGACATCCAGCCTGCCCCCAGTGCAGAAGAGCTGGC
CGAGCAGCTGGACCTGGGTGAGGAGGCATCAGCACTGCTGCTGGAAGACG
GTGAAGCCCCAGCCAGCACCAAGGAGGAGCCCTTGGAGGAGGAAGCAACG
ACCCAGAGCCGGGCTGGAAGGGAAGAAGAGGAGAAGGAGCAGGAGGAGGA
GGGAGATGTGTCTACCCTGGAGCAGGGAGAGGGCAGGCTGCAGCTGCGGG
CCTCGGTGAAGAGAAAAAGCAGGCCCTGCTCCTTGCCTGTGTCCGAGCTG
GAGACGGTGATCGCGTCAGCCTGCGGGGACCCCGAGACCCCGCGGACACA
CTACATCCGCATCCACACCCTGCTGCACAGCATGCCCTCCGCCCAGGGCG
GCAGCGCGGCAGAGGAGGAGGACGGCGCGGAGGAGGAGTCCACCCTCAAG
GACTCCTCGGAGAAGGATGGGCTCAGCGAGGTGGACACGGTGGCCGCTGA
CCCGTCTGCCCTGGAAGAGGACAGAGAAGAGCCCGAGGGGGCTACTCCAG
GCACGGCGCACCCTGGCCACTCCGGGGGCCACTTCCCCAGCCTGGCCAAT
GGCGCGGCCCAGGATGGCGACACGCACCCCAGCACCGGGAGCGAGAGCGA
CTCCAGCCCCAGGCAAGGCGGGGACCACAGTTGCGAGGGCTGTGACGCGT
CCTGCTGCAGCCCCTCGTGCTACAGCTCCTCGTGCTACAGCACGTCCTGC
TACAGCAGCTCGTGCTACAGCGCCTCGTGCTACAGCCCCTCCTGCTACAA
CGGCAACAGGTTCGCCAGCCACACGCGCTTCTCCTCCGTGGACAGCGCCA
AGATCTCCGAGAGCACGGTCTTCTCCTCGCAAGACGACGAGGAGGAGGAG
AACAGCGCGTTCGAGTCGGTACCCGACTCCATGCAGAGCCCTGAGCTGGA
CCCGGAGTCCACGAACGGCGCTGGGCCGTGGCAAGACGAGCTGGCCGCCC
CTAGCGGGCACGTGGAAAGAAGCCCGGAAGGTCTGGAATCCCCCGTGGCA
GGTCCAAGCAATCGGAGAGAAGACTGGGAAGCTCGAATTGACAGCCACGG
GGGGTCTTTTATGTGGACCACGTGAACCGCACAACCACCTGGCAGCGTCC
GACGGCAGCAGCCACCCCGGATGGCATGCGGAGATCGGGGTCCATCCAGC
AGATGGAGCAACTCAACAGGCGGTATCAAAACATTCAGCGAACCATTGCA
ACAGAGAGGTCCGAAGAAGATTCTGGCAGCCAAAGCTGCGAGCAAGCCCC
AGCAGGAGGAGGCGGAGGTGGAGGGAGTGACTCAGAAGCCGAATCTTCCC
AGTCCAGCTTAGATCTAAGGAGAGAGGGGTCACTTTCTCCAGTGAACTCA
CAAAAAATCACCTTGCTGCTGCAGTCCCCAGCGGTCAAGTTCATCACCAA
CCCCGAGTTCTTCACTGTGCTACACGCCAATTATAGTGCCTACCGAGTCT
TCACCAGTAGCACCTGCTTAAAGCACATGATTCTGAAAGTCCGACGGGAT
GCTCGCAATTTTGAACGCTACCAGCACAACCGGGACTTGGTGAATTTCAT
CAACATGTTCGCAGACACTCGGCTGGAACTGCCCCGGGGCTGGGAGATCA
AAACGGACCAGCAGGGAAAGTCTTTTTTCGTGGACCACAACAGTCGAGCT
ACCACTTTCATTGACCCCCGAATCCCTCTTCAGAACGGTCGTCTTCCCAA
TCATCTAACTCACCGACAGCACCTCCAGAGGCTCCGAAGTTACAGCGCCG
GAGAGGCCTCAGAAGTTTCTAGAAACAGAGGAGCCTCTTTACTGGCCAGG
CCAGGACACAGCTTAGTAGCTGCTATTCGAAGCCAACATCAACATGAGTC
ATTGCCACTGGCATATAATGACAAGATTGTGGCATTTCTTCGCCAGCCAA
ACATTTTTGAAATGCTGCAAGAGCGTCAGCCAAGCTTAGCAAGAAACCAC
ACACTCAGGGAGAAAATCCATTACATTCGGACTGAGGGTAATCACGGGCT
TGAGAAGTTGTCCTGTGATGCGGATCTGGTCATTTTGCTGAGTCTCTTTG
AAGAAGAGATTATGTCCTACGTCCCCCTGCAGGCTGCCTTCCACCCTGGG
TATAGCTTCTCTCCCCGATGTTCACCCTGTTCTTCACCTCAGAACTCCCC
AGGTTTACAGAGAGCCAGTGCAAGAGCCCCTTCCCCCTACCGAAGAGACT
TTGAGGCCAAGCTCCGCAATTTCTACAGAAAACTGGAAGCCAAAGGATTT
GGTCAGGGTCCGGGGAAAATTAAGCTCATTATTCGCCGGGATCATTTGTT
GGAGGGAACCTTCAATCAGGTGATGGCCTATTCGCGGAAAGAGCTCCAGC
GAAACAAGCTCTACGTCACCTTTGTTGGAGAGGAGGGCCTGGACTACAGT
GGCCCCTCGCGGGAGTTCTTCTTCCTTCTGTCTCAGGAGCTCTTCAACCC
TTACTATGGACTCTTTGAGTACTCGGCAAATGATACTTACACGGTGCAGA
TCAGCCCCATGTCCGCATTTGTAGAAAACCATCTTGAGTGGTTCAGGTTT
AGCGGTCGCATCCTGGGTCTGGCTCTGATCCATCAGTACCTTCTTGACGC
TTTCTTCACGAGGCCCTTCTACAAGGCACTCCTGAGACTGCCCTGTGATT
TGAGTGACCTGGAATATTTGGATGAGGAATTCCACCAGAGTTTGCAGTGG
ATGAAGGACAACAACATCACAGACATCTTAGACCTCACTTTCACTGTTAA
TGAAGAGGTTTTTGGACAGGTCACGGAAAGGGAGTTGAAGTCTGGAGGAG
CCAACACACAGGTGACGGAGAAAAACAAGAAGGAGTACATCGAGCGCATG
GTGAAGTGGCGGGTGGAGCGCGGCGTGGTACAGCAGACCGAGGCGCTGGT
GCGCGGCTTCTACGAGGTTGTAGACTCGAGGCTGGTGTCCGTGTTTGATG
CCAGGGAGCTGGAGCTGGTGATAGCTGGCACCGCGGAAATCGACCTAAAT
GACTGGCGGAATAACACTGAGTACCGGGGAGGTTACCACGATGGGCATCT
TGTGATCCGCTGGTTCTGGGCTGCGGTGGAGCGCTTCAATAATGAGCAGA
GGCTGAGATTACTGCAGTTTGTCACGGGAACATCCAGCGTGCCCTACGAA
GGCTTCGCAGCCCTCCGTGGGAGCAATGGGCTTCGGCGCTTCTGCATAGA
GAAATGGGGGAAAATTACTTCTCTCCCCAGGGCACACACATGCTTCAACC
GACTGGATCTTCCACCGTATCCCTCGTACTCCATGTTGTATGAAAAGCTG
TTAACAGCAGTAGAGGAAACCAGCACCTTTGGACTTGAGTGAGGACATGG
AACCTCGCCTGACATTTTCCTGGCCAGTGACATCACCCTTCCTGGGATGA
TCCCCTTTTCCCTTTCCCTTAATCAACTCTCCTTTGATTTTGGTATTCCA
TGATTTTTATTTTCAAAC

Human NEDL2 nucleic acid sequence (uniprot.org/uniprot/Q9P2P5):

(SEQ ID NO: 56)
AGAGTTCCATCAGAGCCTGCAGTGGATGAAAGACAATGATATCCATGACA
TCCTAGACCTCACGTTCACTGTGAACGAAGAAGTATTTGGGCAGATAACT
GAACGAGAATTAAAGCCAGGGGGTGCCAATATCCCAGTTACAGAGAAGAA
CAAGAAGGAGTACATCGAGAGGATGGTGAAGTGGAGGATTGAGAGGGGTG
TTGTACAGCAAACAGAGAGCTTAGTGCGTGGCTTCTATGAGGTGGTGGAT
GCCAGGCTGGTATCTGTTTTTGATGCAAGAGAACTGGAATTGGTCATCGC
AGGCACAGCTGAAATAGACCTAAGTGATTGGAGAAACAACACAGAATATA
GAGGAGGATACCATGACAATCATATTGTAATTCGGTGGTTCTGGGCTGCA
GTGGAAAGATTCAACAATGAACAACGACTAAGGTTGTTACAGTTTGTTAC
AGGCACATCCAGCATTCCCTATGAAGGATTTGCTTCACTCCGAGGGAGTA
ACGGCCCAAGAAGATTCTGTGTGGAGAAATGGGGGAAAATCACTGCTCTT
CCCAGAGCGCATACATGTTTTAACCGTCTGGATCTGCCTCCCTACCCATC
CTTTTCCATGCTTTATGAAAAACTGTTGACAGCAGTTGAAGAAACCAGTA
CTTTTGGACTTGAGTGACCTGGAAGCTGAATGCCCATCTCTGTGGACAGG
CAGTTTCAGAAGCTGCCTTCTAGAAGAATGATTGAACATTGGAAGTTTCA
AGAGGATGCTTCCTTTAGGATAAAGCTACGTGCTGTTGTTTTCCAGGAAC
AAGTGCTCTGTCACATTTGGGGACTGGAGATGAGTCCTCTTGGAAGGATT
TGGGTGAGCTTGATGCCCAGGGAACAACCCAACCGTCTTTCAATCAACAG
TTCTTGACTGCCAAACTTTTTCCATTTGTTATGTTCCAAGACAAAGATGA
ACCCATACATGATCAGCTCCACGGTAATTTTTAGGGACTCAGGAGAATCT
TGAAACTTACCCTTGAACGTGGTTCAAGCCAAACTGGCAGCATTTGGCCC
AATCTCCAAATTAGAGCAAGTTAAATAATATAATAAAAGTAAATATATTT
CCTGAAAGTACATTCATTTAAGCCCTAAGTTATAACAGAATATTCATTTC
TTGCTTATGAGTGCCTGCATGGTGTGCACCATAGGTTTCCGCTTTCATGG
GACATGAGTGAAAATGAAACCAAGTCAATATGAGGTACCTTTACAGATTT
GCAATAAGATGGTCTGTGACAATGTATATGCAAGTGGTATGTGTGTAATT
ATGGCTAAAGACAAACCATTATTCAGTGAATTACTAATGACAGATTTTAT
GCTTTATAATGCATGAAAACAATTTTAAAATAACTAGCAATTAATCACAG
CATATCAGGAAAAAGTACACAGTGAGTTCTGTTTATTTTTTGTAGGCTCA
TTATGTTTATGTTCTTTAAGATGTATATAAGAACCTACTTATCATGCTGT
ATGTATCACTCATTCCATTTTCATGTTCCATGCATACTCGGGCATCATGC
TAATATGTATCCTTTTAAGCACTCTCAAGGAAACAAAAGGGCCTTTTATT
TTTATAAAGGTAAAAAAAATTCCCCAAATATTTTGCACTGAATGTACCAA
AGGTGAAGGGACATTACAATATGACTAACAGCAACTCCATCACTTGAGAA
GTATAATAGAAAATAGCTTCTAAATCAAACTTCCTTCACAGTGCCGTGTC
TACCACTACAAGGACTGTGCATCTAAGTAATAATTTTTTAAGATTCACTA
TATGTGATAGTATGATATGCATTTATTTAAAATGCATTAGACTCTCTTCC
ATCCATCAAATACTTTACAGGATGGCATTTAATACAGATATTTCGTATTT
CCCCCACTGCTTTTTATTTGTACAGCATCATTAAACACTAAGCTCAGTTA
AGGAGCCATCAGCAACACTGAAGAGATCAGTAGTAAGAATTCCATTTTCC
CTCATCAGTGAAGACACCACAAATTGAAACTCAGAACTATATTTCTAAGC
CTGCATTTTCACTGATGCATAATTTTCTTATTAATATTAAGAGACAGTTT
TTCTATGGCATCTCCAAAACTGCATGACATCACTAGTCTTACTTCTGCTT
AATTTTATGAGAAGGTATTCTTCATTTTAATTGCTTTTGGGATTACTCCA
CATCTTTGTTTATTTCTTGACTAATCAGATTTTCAATAGAGTGAAGTTAA
ATTGGGGGTCATAAAAGCATTGGATTGACATATGGTTTGCCAGCCTATGG
GTTTACAGGCATTGCCCAAACATTTCTTTGAGATCTATATTTATAAGCAG
CCATGGAATTCCTATTATGGGATGTTGGCAATCTTACATTTTATAGAGGT
CATATGCATAGTTTTCATAGGTGTTTTGTAAGAACTGATTGCTCTCCTGT
GAGTTAAGCTATGTTTACTACTGGGACCCTCAAGAGGAATACCACTTATG
TTACACTCCTGCACTAAAGGCACGTACTGCAGTGTGAAGAAATGTTCTGA
AAAAGGGTTATAGAAATCTGGAAATAAGAAAGGAAGAGCTCTCTGTATTC
TATAATTGGAAGAGAAAAAAAGAAAAACTTTTAACTGGAAATGTTAGTTT
GTACTTATTGATCATGAATACAAGTATATATTTAATTTTGCAAAAAAAAA
AAAAAAAAAAAAAAG

Some aspects of this invention provide expression constructs that encode any of the proteins, nucleic acids, such as RNAs, or fusions thereof described herein.

Nucleic acids encoding any of the proteins and/or nucleic acid (including RNA) described herein, may be in any number of nucleic acid “vectors” known in the art. As used herein, a “vector” means any nucleic acid or nucleic acid-bearing particle, cell, or organism capable of being used to transfer a nucleic acid into a host cell. The term “vector” includes both viral and nonviral products and means for introducing the nucleic acid into a cell. A “vector” can be used in vitro, ex vivo, or in vivo. Non-viral vectors include plasmids, cosmids, artificial chromosomes (e.g., bacterial artificial chromosomes or yeast artificial chromosomes) and can comprise liposomes, electrically charged lipids (cytofectins), DNA-protein complexes, and biopolymers, for example. Viral vectors include retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also comprise the entire genome sequence or recombinant genome sequence of a virus. A vector can also comprise a portion of the genome that comprises the functional sequences for production of a virus capable of infecting, entering, or being introduced to a cell to deliver nucleic acid therein.

Expression of any of the proteins and/or nucleic acids (including RNA) described herein, may be controlled by any regulatory sequence (e.g., a promoter sequence) known in the art. Regulatory sequences, as described herein, are nucleic acid sequences that regulate the expression of a nucleic acid sequence. A regulatory or control sequence may include sequences that are responsible for expressing a particular nucleic acid or may include other sequences, such as heterologous, synthetic, or partially synthetic sequences. The sequences can be of eukaryotic, prokaryotic, or viral origin that stimulate or repress transcription of a gene in a specific or non-specific manner and in an inducible or non-inducible manner. Regulatory or control regions may include origins of replication, RNA splice sites, introns, chimeric or hybrid introns, promoters, enhancers, transcriptional termination sequences, poly A sites, locus control regions, signal sequences that direct the polypeptide into the secretory pathways of the target cell. A heterologous regulatory region is a regulatory region not naturally associated with the expressed nucleic acid it is linked to. Included among the heterologous regulatory regions are regulatory regions from a different species, regulatory regions from a different gene, hybrid regulatory sequences, and regulatory sequences that do not occur in nature, but which are designed by one of ordinary skill in the art.

The term operably linked refers to an arrangement of sequences or regions wherein the components are configured so as to perform their usual or intended function. Thus, a regulatory or control sequence operably linked to a coding sequence is capable of affecting the expression of the coding sequence. The regulatory or control sequences need not be contiguous with the coding sequence, so long as they function to direct the proper expression or polypeptide production. Thus, for example, intervening untranslated but transcribed sequences can be present between a promoter sequence and the coding sequence, and the promoter sequence can still be considered operably linked to the coding sequence. A promoter sequence, as described herein, is a DNA regulatory region a short distance from the 5′ end of a gene that acts as the binding site for RNA polymerase. The promoter sequence may bind RNA polymerase in a cell and/or initiate transcription of a downstream (3′ direction) coding sequence. The promoter sequence may be a promoter capable of initiating transcription in prokaryotes or eukaryotes. Some non-limiting examples of eukaryotic promoters include the cytomegalovirus (CMV) promoter, the chicken β-actin (CBA) promoter, and a hybrid form of the CBA promoter (CBh).

### Microvesicle-Producing Cells

A microvesicle-producing cell of the present invention may be a cell containing any of the expression constructs, any of the fusion proteins, or any of the agents (e.g., small molecules, proteins, and nucleic acids (e.g., DNA, RNA), DNA plasmids, siRNA, mRNA, Cas9 and other Cas proteins, zinc finger nucleases, TALENs, etc.) described herein. For example, in some aspect, the present disclosure provides microvesicle-producing cells comprising: a first isolated nucleic acid encoding an ARRDC1 protein or a variant thereof, a second isolated nucleic acid encoding a Nipah virus (NiV) glycoprotein variant fused to an antibody or antigen-binding fragment, wherein the NiV glycoprotein variant does not recognize and/or bind to an ephrin receptor, and a third isolated nucleic acid encoding a NiV fusion protein. In some embodiments, the first isolated nucleic acid, the second isolated nucleic acid, and the third isolated nucleic acid are expressed on the same recombinant expression construct under the control of one or more heterologous promoters. In some embodiments, the first isolated nucleic acid, the second isolated nucleic acid, and the third isolated nucleic acid are expressed on more than one recombinant expression construct, wherein the one or more recombinant expression constructs are under the control of one or more heterologous promoters. In certain embodiments, the first isolated nucleic acid, the second isolated nucleic acid, and the third isolated nucleic acid are expressed on different recombinant expression constructs, wherein the different recombinant expression constructs are each under the control of a heterologous promoter.

Any of the expression constructs described herein may be stably inserted into the genome of the cell. In some embodiments, the expression construct is maintained in the cell, but not inserted into the genome of the cell. In some embodiments, the expression construct is in a vector, for example, a plasmid vector, a cosmid vector, a viral vector, or an artificial chromosome. In some embodiments, the expression construct further comprises additional sequences or elements that facilitate the maintenance and/or the replication of the expression construct in the microvesicle-producing cell, or that improve the expression of the fusion protein in the cell. Such additional sequences or elements may include, for example, an origin of replication, an antibiotic resistance cassette, a polyA sequence, and/or a transcriptional isolator. Some expression constructs suitable for the generation of microvesicle producing cells according to aspects of this invention are described elsewhere herein. Methods and reagents for the generation of additional expression constructs suitable for the generation of microvesicle producing cells according to aspects of this invention will be apparent to those of skill in the art based on the present disclosure. In some embodiments, the microvesicle producing cell is a mammalian cell, for example, a mouse cell, a rat cell, a hamster cell, a rodent cell, or a nonhuman primate cell. In some embodiments, the microvesicle producing cell is a human cell.

One skilled in the art may employ conventional techniques, such as molecular or cell biology, virology, microbiology, and recombinant DNA techniques. Exemplary techniques are explained fully in the literature. For example, one may rely on the following general texts to make and use the invention: Sambrook et al., Molecular Cloning: A Laboratory Manual, Second Edition (1989) Cold Spring Harbor Laboratory Press, Cold Spring Harbor, New York, and Sambrook et al., Third Edition (2001); DNA Cloning: A Practical Approach, Volumes I and II (D. N. Glover ed. 1985); Oligonucleotide Synthesis (M. J. Gaited. 1984); Nucleic Acid Hybridization (B. D. Hames & S. J. Higgins eds. (1985)); Transcription And Translation Hames & Higgins, eds. (1984); Animal Cell Culture (RI. Freshney, ed. (1986)); Immobilized Cells And Enzymes (IRL Press, (1986)); Gennaro et al., (eds.) Remington's Pharmaceutical Sciences, 18th edition; B. Perbal, A Practical Guide To Molecular Cloning (1984); F. M. Ausubel et al., (eds.), Current Protocols in Molecular Biology, John Wiley & Sons, Inc. (updates through 2001), Coligan et al., (eds.), Current Protocols in Immunology, John Wiley & Sons, Inc. (updates through 2001); W. Paul et al., (eds.) Fundamental Immunology, Raven Press; E. J. Murray et al., (ed.) Methods in Molecular Biology: Gene Transfer and Expression Protocols, The Humana Press Inc. (1991) (especially vol. 7); and J. E. Celis et al., Cell Biology: A Laboratory Handbook, Academic Press (1994).

### Delivery of ARMMs

The inventive microvesicles (e.g., ARMMs modified with NiV glycoproteins and fusion proteins and containing any of the agents described herein), comprise an antibody or antibody binding fragment, which may be used to target the delivery of ARMMs to specific cell types, resulting in the release of the contents of the ARMM into the cytoplasm of the specific targeted cell type. Antibodies or antigen-binding fragments contemplated for use in the present disclosure include, but are not limited to, single-chain variable fragments (scFvs) nanobodies and DARPins. The antibody or antigen-binding fragment may allow for the targeting of particular cell types (e.g., neurons, astrocytes, oligodendrocytes, microglial cells, T-cells, dendritic cells, B cells, NK cells, stem cells, progenitor cells, endothelial cells, muscle cells, myocardial cells, epithelial cells, and hepatic cells). In certain embodiments, the antibody or antigen-binding fragment targets T cells. In certain embodiments, the antibody or antigen-binding fragment targets neurons.

Some aspects of this invention relate to the recognition that ARMMs modified with engineered NiV glycoproteins and fusion proteins are taken up by target cells (e.g., T cell, neurons, or other specific cell types), and ARMM uptake results in the release of the contents of the ARMM (e.g., any of the agents described herein) into the cytoplasm of the target cells. In some embodiments, the payload is an agent that affects a desired change in the target cell, for example, a change in cell survival, proliferation rate, a change in differentiation stage, a change in a cell identity, a change in chromatin state, a change in the transcription rate of one or more genes, a change in the transcriptional profile, or a post-transcriptional change in gene compression of the target cell. It will be understood by those of skill in the art that the agent to be delivered will be chosen according to the desired effect in the target cell.

Thus, in some aspects, the present disclosure provides methods of delivering a molecule to a target cell comprising contacting the target cell with any of the microvesicles described herein. In some embodiments, the target cell is any of the cell types described herein (e.g., T cells or neurons). In some embodiments, the microvesicle is delivered to a target cell in a subject (e.g., a patient). In some aspects, the present disclosure provides methods of treating a patient comprising administering to the patient any of the microvesicles described herein, or any of the microvesicle-producing cells described herein. In some aspects, the present disclosure provides methods of delivering an ARMM comprising delivering any of the ARMMs or microvesicle-producing cells described herein to a subject. In some embodiments, the subject is mammalian. In certain embodiments, the subject is human.

In some embodiments, cells from a subject are obtained, and an agent is delivered to the cells by a microvesicle or method provided herein ex vivo. In some embodiments, the treated cells are selected for those cells in which a desired gene is expressed or repressed. In some embodiments, treated cells carrying a protein or RNA agent are returned to the subject they were obtained from.

Target cells can be contacted with an ARMM in different ways. For example, a target cell may be contacted directly with an ARMM as described herein, or with an isolated ARMM from a microvesicle-producing cell. The contacting can be done in vitro by administering the ARMM to the target cell in a culture dish, or in vivo by administering the ARMM to a subject (e.g., parenterally or non-parenterally). In some embodiments, an ARMM is produced from a cell obtained from a subject. In some embodiments, the ARMM that was produced from a cell that was obtained from the subject is administered to the subject from which the ARMM producing cell was obtained. In some embodiments, the ARMM that was produced from a cell that was obtained from the subject is administered to a subject different from the subject from which the ARMM producing cell was obtained. As one example, a cell may be obtained from a subject and engineered to express one or more of the constructs provided herein (e.g., any of the polynucleotides encoding the various components of the ARMMs described herein). The cell obtained from the subject and engineered to express one or more of the constructs provided herein may be administered to the same subject, or a different subject, from which the cell was obtained. Alternatively, the cell obtained from the subject and engineered to express one or more of the constructs provided herein produces ARMMs, which may be isolated and administered to the same subject from which the cell was obtained or administered to a different subject from which the cell was obtained.

Alternatively, a target cell can be contacted with a microvesicle producing cell as described herein, for example, in vitro by co-culturing the target cell and the microvesicle producing cell, or in vivo by administering a microvesicle producing cell to a subject harboring the target cell. Accordingly, the method may include contacting the target cell with a microvesicle, for example, an ARMM containing any of the agents described herein. The target cell may be contacted with a microvesicle-producing cell, as described herein, or with an isolated microvesicle that has a lipid bilayer, an ARRDC1 protein or variant thereof, and a NiV glycoprotein and NiV fusion protein.

It should be appreciated that the target cell may be of any origin, for example, from an organism. In some embodiments, the target cell is a mammalian cell. Some non-limiting examples of a mammalian cell include, without limitation, a mouse cell, a rat cell, a hamster cell, a rodent cell, and a nonhuman primate cell. In some embodiments, the target cell is a human cell. It should also be appreciated that the target cell may be of any cell type, such as a cell type of the nervous system. In other cases, the target cell may be any differentiated cell type found in a subject. In some embodiments, the target cell is a cell in vitro, and the method includes administering the microvesicle to the cell in vitro, or co-culturing the target cell with the microvesicle-producing cell in vitro. In some embodiments, the target cell is a cell in a subject, and the method comprises administering the microvesicle or the microvesicle-producing cell to the subject. In some embodiments, the subject is a mammalian subject, for example, a rodent, a mouse, a rat, a hamster, or a non-human primate. In some embodiments, the subject is a human subject.

In some embodiments, the target cell is a pathological cell. In some embodiments, the target cell is a cancer cell. In some embodiments, the microvesicle is associated with an antibody or antigen-binding fragment that selectively binds an antigen on the surface of the target cell. In some embodiments, the antigen of the target cell is a cell surface antigen. In some embodiments, the antibody or antigen-binding fragment is a single-chain variable fragment (scFv), a nanobody, or a DARPin. Suitable surface antigens of target cells, for example, of specific target cell types, e.g., T cells or neurons, are known to those of skill in the art, as are suitable antibodies or antigen-binding fragments that specifically bind such antigens. Methods for producing membrane-bound binding agents, for example, membrane-bound immunoglobulins, membrane-bound antibodies, or antibody fragments that specifically bind a surface antigen expressed on the surface of a target cell, are also known to those of skill in the art. The choice of the antibody or antigen-binding fragment will depend, of course, on the identity or the type of target cell. Cell surface antigens specifically expressed on various types of target cells that can be targeted by ARMMs comprising membrane-bound binding agents will be apparent to those of skill in the art. It will be appreciated that the present invention is not limited in this respect. In some embodiments, the target cells express the antigen that antibody or antigen-binding fragment binds to, for example that the antigen that antibody or antigen-binding fragment selectively binds to. In some embodiments, the target cells express the antigen that antibody or antigen-binding fragment on the surface of the target cell.

In some embodiments, any of the ARMMs described herein further include a detectable label. Such ARMMs allow for the labeling of a target cell without genetic manipulation. Detectable labels suitable for direct delivery to target cells are known in the art, and include, but are not limited to, fluorescent proteins, fluorescent dyes, membrane-bound dyes, and enzymes, for example, membrane-bound or cytosolic enzymes, catalyzing a reaction resulting in a detectable reaction product. Detectable labels suitable according to some aspects of this invention further include membrane-bound antigens, for example, membrane-bound ligands that can be detected with commonly available antibodies or antigen-binding agents.

In some embodiments, ARMMs are provided that comprise an RNA agent that encodes a transcription factor, a transcriptional repressor, a fluorescent protein, a kinase, a phosphatase, a protease, a ligase, a chromatin modulator, or a recombinase. In some embodiments, ARMMs are provided that comprise an RNA agent (e.g., an siRNA) that inhibits expression of a transcription factor, a transcriptional repressor, a fluorescent protein, a kinase, a phosphatase, a protease, a ligase, a chromatin modulator, or a recombinase. In some embodiments, the RNA agent is a therapeutic RNA. In some embodiments the RNA agent is an RNA that effects a change in the state or identity of a target cell. For example, in some embodiments, the RNA agent encodes a reprogramming factor. Suitable transcription factors, transcriptional repressors, fluorescent proteins, kinases, phosphatases, proteases, ligases, chromatin modulators, recombinases, and reprogramming factors may be encoded by an RNA agent that is associated with a binding RNA to facilitate their incorporation into ARMMs, and their function may be tested by any methods that are known to those skilled in the art, and the invention is not limited in this respect.

Methods for isolating the ARMMs described herein (i.e., ARMMs modified with NiV glycoproteins and fusion proteins) are also provided by the present disclosure. One exemplary method includes collecting the culture medium, or supernatant, of a cell culture comprising microvesicle-producing cells. In some embodiments, the cell culture comprises cells obtained from a subject, for example, cells suspected to exhibit a pathological phenotype, for example, a hyperproliferative phenotype. In some embodiments, the cell culture comprises genetically engineered cells producing ARMMs, for example, cells expressing a recombinant ARMM protein, for example, a recombinant ARRDC1 or TSG101 protein, such as an ARRDC1 or TSG101 protein fused to an RNA or protein agent as described herein. In some embodiments, the supernatant is pre-cleared of cellular debris by centrifugation, for example, by two consecutive centrifugations of increasing G value (e.g., 500G and 2000G). In some embodiments, the method comprises passing the supernatant through a 0.2 μm filter, eliminating all large pieces of cell debris and whole cells. In some embodiments, the supernatant is subjected to ultracentrifugation, for example, at 120,000G for 2 hours, depending on the volume of centrifugate. The pellet obtained comprises microvesicles. In some embodiments, exosomes are depleted from the microvesicle pellet by staining and/or sorting (e.g., by FACS or MACS) using an exosome marker as described herein. Isolated or enriched ARMMs can be suspended in culture media or a suitable buffer, as described herein.

### Pharmaceutical Compositions

Other aspects of the present disclosure relate to pharmaceutical compositions comprising any of the ARMMs or microvesicle-producing cells provided herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds as described herein).

As used herein, the term “pharmaceutically-acceptable carrier” means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue, or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials that can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates, and/or polyanhydrides; (22) bulking agents, such as polypeptides and amino acids (23) serum components, such as serum albumin, HDL, and LDL; (22) C₂-C₁₂ alcohols, such as ethanol; and (24) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. The terms such as “excipient”, “carrier”, “pharmaceutically acceptable carrier,” or the like are used interchangeably herein.

In some embodiments, the pharmaceutical composition is formulated for delivery to a subject, e.g., for delivering an agent to a cell. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical composition can also include a solubilizing agent and a local anesthetic such as lidocaine to ease pain at the site of the injection. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's solution, or Hank's solution. In addition, the pharmaceutical composition can be in solid forms and re-dissolved or suspended immediately prior to use. Lyophilized forms are also contemplated.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent, i.e., carrier, or vehicle.

### Kits, Vectors, Cells

Further, the pharmaceutical composition can be provided as a pharmaceutical kit comprising (a) a container containing any of the ARMMs or microvesicle-producing cells described herein and (b) a second container containing a pharmaceutically acceptable diluent (e.g., sterile water) for injection. The pharmaceutically acceptable diluent can be used, e.g., for reconstitution or dilution of the ARMM or microvesicle-producing cell. Optionally associated with such container(s) can be a notice in the form prescribed by a governmental agency regulating the manufacture, use or sale of pharmaceuticals or biological products, which notice reflects approval by the agency of manufacture, use, or sale for human administration.

In another aspect, an article of manufacture containing materials useful for the treatment of a disease or disorder is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is an ARMM (or microvesicle-producing cell) of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

Some aspects of this disclosure provide kits comprising a polynucleotide encoding any of the components of the ARMMs provided herein. In some embodiments, the polynucleotide encodes any of the proteins, fusion proteins, and/or RNAs provided herein. In some embodiments, the polynucleotide comprises a heterologous promoter that drives expression of any of the proteins, fusion proteins, and/or RNAs provided herein.

Some aspects of this disclosure provide microvesicle (e.g., ARMM) producing cells comprising any of the proteins, fusion proteins, and/or RNAs provided herein. In some embodiments, the cells comprise a polynucleotide that encodes any of the proteins, fusion proteins, and/or RNAs provided herein. In some embodiments, the cells comprise any of the polynucleotides or vectors provided herein. In some embodiments, the vector comprise viral targeting proteins.

It should be appreciated however, that additional proteins, fusion proteins, and RNAs would be apparent to the skilled artisan based on the present disclosure and knowledge in the art.

The function and advantage of these and other embodiments of the present invention will be more fully understood from the Examples below. The following Examples are intended to illustrate the benefits of the present invention and to describe particular embodiments but are not intended to exemplify the full scope of the invention. Accordingly, it will be understood that the Examples are not meant to limit the scope of the invention.

### EXAMPLES

### Example 1: Targeted Delivery of ARMMs to CD8⁺ T Cells

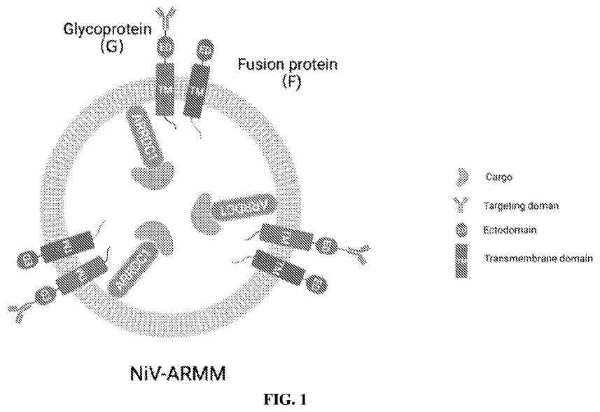

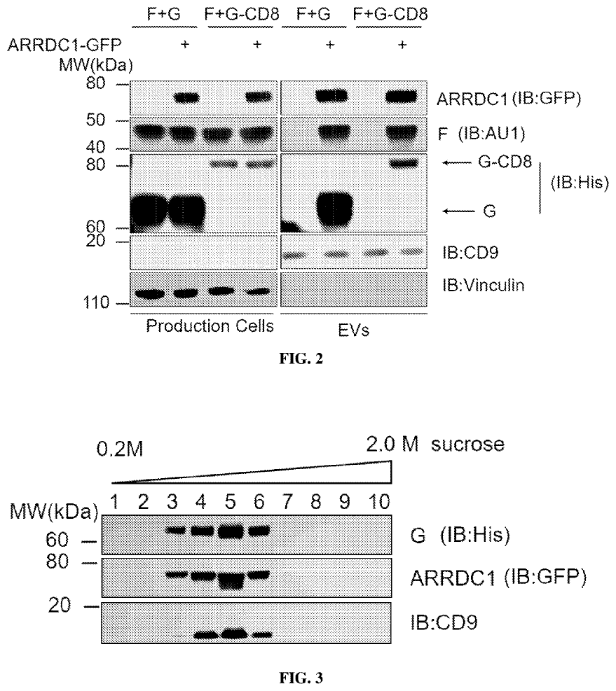

NiV-F and-G proteins can be used to pseudotype lentiviruses to allow specific cell targeting (R. Bender et al., PLOS Genetics, 2016). It was analyzed whether the NiV F/G system could be used to modify non-viral, lipid-bilayer-encapsulated particles such as ARMMs. First, it was tested whether NiV-F/G proteins can be recruited and packaged onto ARMMs. When co-expressed with ARRDC1 (which drives the formation and budding of ARMMs) and its associated cargos, NiV-F/G proteins can be incorporated onto ARMMs, thus allowing for targeting of the vesicles to specific cells or tissues (FIG. 1). Two NiV-G constructs were used for comparison: NiV-G-NT for non-targeting and NiV-G-CD8 that targets CD8 protein. Both constructs contain mutations that result in the loss of binding and recognition of the G protein's natural receptors (ephrins). NiV-G-CD8 contains a fusion to CD8-targeting scFv, which recognizes and binds specifically to CD8. For NiV-F, a truncation variant with improved fusion activity was used. NiV-G-NT (or NiV-G-CD8) and NiV-F were transfected into HEK293T cells with or without ARRDC1-GFP. As shown by Western blotting (FIG. 2), although NiV-F protein was expressed well in all cells, in extracellular vesicles, it was present only when co-expressed with ARRDC1-GFP. Similarly, NiV-G protein was present only in EVs collected from ARRDC1-co-transfected cells. The pattern is the same for both NiV-G-NT and NiV-G-CD8. These results indicate that the presence of NiV-G/F proteins in EVs is dependent on the co-expression of ARRDC1, suggesting that these NiV proteins are incorporated into ARMMs. Fractionation of the EVs by density gradient ultracentrifugation showed that NiV-G co-segregates well with ARRDC1 (FIG. 3), providing further evidence for the incorporation of NiV proteins into ARMMs.

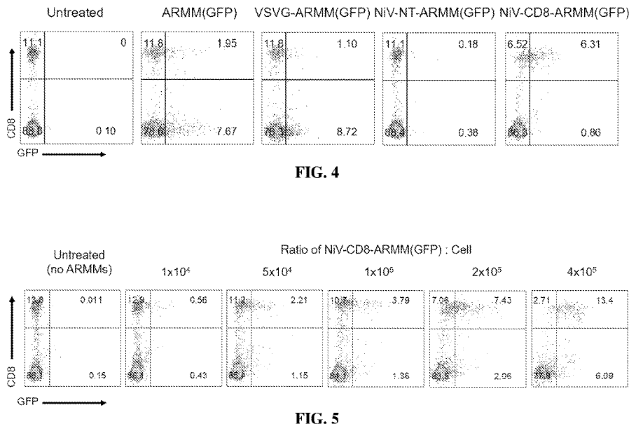

It was next assessed whether ARMMs incorporated with NiV proteins mediate specific cell targeting. In particular, it was assessed whether NiV-G-CD8-containing ARMMs can specifically target CD8⁺ cells. PBMCs, which contain a population of CD8⁺ T cells, were used for this analysis. Primary PBMCs were incubated with either NiV-G-NT-ARMMs or NiV-G-CD8-ARMMs. Unmodified ARMMs and VSVG-pseudotyped ARMMs were used as controls. All ARMMs contained GFP protein as the cargo. After incubation with ARMMs, PBMCs were stained for CD8 and subjected to flow cytometry analysis to assess CD8 staining and cargo (GFP) uptake. As shown in FIG. 4, PBMCs contained 11-12% CD8⁺ cells. Incubation of PBMCs with unmodified ARMMs or VSVG-ARMMs led to a slight increase in the percentage of GFP-positive cells in both the CD8⁺ and CD8⁻ cell populations. Incubation with NiV-NT-ARMMs did not lead to much increase in GFP-positive cells in either CD8⁺ or CD8⁻ populations. In contrast, incubation of PBMCs with NiV-CD8-ARMMs resulted in GFP positivity in about half of the CD8⁺-cell population. Only a negligible percentage (0.86%) of CD8⁻ cells became GFP-positive. This result demonstrated a specific delivery of GFP protein into CD8⁺ cells by ARMMs that were decorated with CD8-targeting NiV proteins. Further experiments showed a dose-dependent delivery of GFP protein into CD8⁺ PBMCs via NiV-CD8-ARMMs (FIG. 5). Although incubation with a very high dosage of ARMMs slightly increased the uptake in CD8 cells, there was a much larger increase in uptake in CD8⁺ cells.

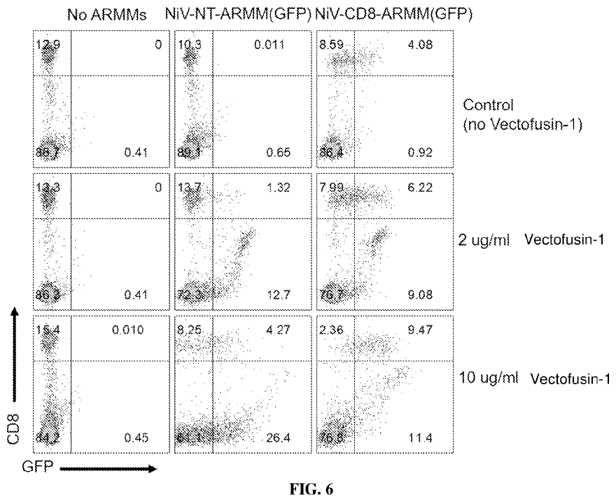

Positively charged peptides such as Vecotfusion-1 can be used to enhance the transduction efficiency of lentiviruses. It was next tested whether Vectofusin-1 can enhance ARMMs delivery into PBMCs. As shown in FIG. 6, Vectofusin-1 significantly increased the uptake of GFP via NiV-CD8-ARMM in CD8⁺ cells. However, the enhancement effect of Vectofusin-1 similarly increased the uptake of GFP in CD8⁻ cells and may be non-specific. In addition, Vectofusin-1 also increased the uptake of GFP even when PBMCs were incubated with NiV-NT-ARMMs.

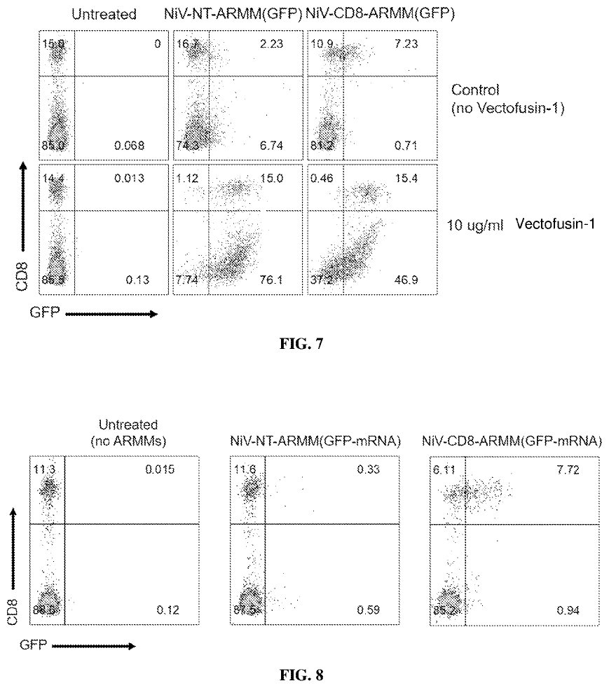

The delivery of ARMMs in activated PBMCs was also tested. Activation of PBMCs by IL2, CD3, and CD28 antibodies leads to the expansion of T cells. Similar to what was observed in unstimulated PBMCs, there was a specific increase in the percentage of GFP-positive cells in the CD8⁺ cell population of activated PBMCs after incubation with NiV-CD8-ARMMs (FIG. 7). Activated PBMCs exhibited an increase in non-specific GFP uptake via NiV-NT-ARMMs as compared to un-activated PBMCs. Vectofusin-1non-specifically enhanced the delivery of GFP via both NiV-NT- and NiV-CD8-ARMMs, and by both CD8⁺ and CD8⁻ cells.

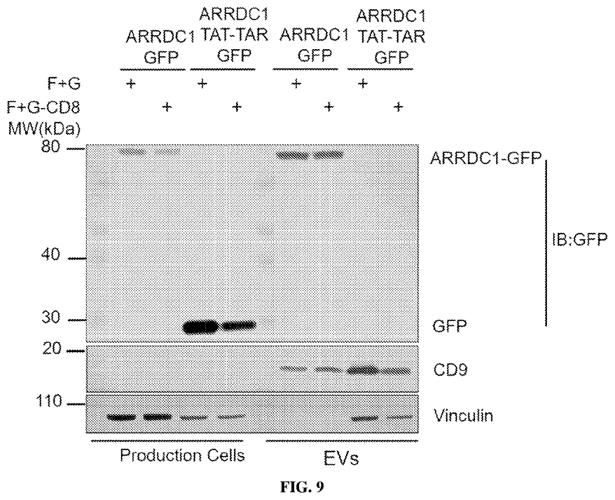

It was further tested whether NiV-ARMMs can mediate targeted delivery of mRNAs. GFP-mRNA was packaged into ARMMs using the published TAT-TAR system, which consists of a short TAT peptide fused directly to the C-terminus of ARRDC1 and TAR fused directly to the 5′ end of GFP mRNA. Binding of TAR to TAT-ARRDC1 allows the recruitment of the TAR-fused mRNA into ARMMs. PBMCs were incubated with either NiV-NT-ARMM (GFPmRNA) or NiV-CD8-ARMM (GFPmRNA), and then subjected to flow cytometry. As shown in FIG. 8, over half of CD8⁺-PBMCs became GFP-positive when incubated with NiV-CD8-ARMM (GFPmRNA), whereas there was close-to-background level of GFP-positive cell population in CD8-negative PBMCs or in PBMCs incubated with NiV-NT-ARMMs. To exclude the possibility that the GFP signal in CD8⁺ cells comes from the carryover of GFP protein, Western blotting was performed to check GFP protein in GFP-mRNA-containing ARMMs. As shown in FIG. 9, although GFP protein is well expressed in production cells, no GFP protein was detected in GFP-mRNA ARMMs. Together, these results indicate that GFP mRNA was specifically delivered to CD8⁺ cells and, furthermore, that the GFP mRNA was functionally translated into GFP protein in the target cells.

### Methods

PBMC culturing and activation. Primary PBMCs were purchased from Lonza and cultured in RPMI 1640 supplemented with 10% FCS, 2 mM L-glutamine, 0.5% streptomycin/penicillin, 25 mM HEPES (Sigma-Aldrich, Munich, Germany), 100 U/ml interleukin-2 (R&D Systems, Minneapolis, USA). For activation, PBMCs were cultured for 72 h in RPMI 1640 supplemented with 10% FCS, 2 mM L-glutamine, 0.5% streptomycin/penicillin, 25 mM HEPES (Sigma-Aldrich, Munich, Germany), 100 U/ml interleukin-2 (R&D Systems, Minneapolis, USA), 1 μg/ml CD3 antibody (clone: OKT3, eBioscience, San Diego, USA) and 1 μg/ml CD28 antibody (clone: CD28.2, eBioscience, San Diego, USA). Following activation, cells were grown in the same medium without the CD3 and CD28 antibodies.

ARMMs production and purification. Briefly, HEK293T cells were transfected with different DNA constructs as indicated using the FuGene 6 reagent (Promega). 24 hours later, the medium was replaced with fresh DMEM supplemented with 10% exo-free FBS. 48 hours after transfection, a first cell culture supernatant was collected, and 72 hours after transfection, a second cell culture supernatant was collected. The collected supernatants were pooled and subjected to two consecutive rounds of centrifugation (500×g and 2000×g). The medium was then passed through a 200 nm filter (Acrodisc) and subjected to ultracentrifugation using the SW41Ti rotor in a L8-M or XE90 centrifuge (Beckman) at 166, 900×g for 2 h. The medium was then aspirated, and the pellets enriched with ARMMs were washed twice with ice-old PBS and resuspended in PBS. NanoSight Nanoparticle tracking analysis is done to determine the number of vesicles in the samples.

Western blotting analysis. Cells were lysed in NP-40 lysis buffer (0.5% NP-40, 50 mM Tris-HCl, and 150 mM NaCl) supplemented with a protease inhibitor mixture (Roche). Lysates or EVs resuspended in lithium dodecyl sulfate sampling buffer (Novex) were resolved on a 4-12% NuPAGE gel (Novex) and transferred onto a PVDF membrane (Bio-Rad). Blots were probed with primary antibodies in Tris-buffered saline containing 0.1% Tween 20 and 5% Nonfat milk, followed by HRP-conjugated anti-rabbit antibody (Cell Signaling, 7074S, at 1:5000 dilution) or anti-mouse antibodies (Cell Signaling, 7076S, at 1:5000 dilution). Primary antibodies used include anti-ARRDC1 rabbit polyclonal antibody (in house, 1:2000), anti-vinculin rabbit monoclonal antibody (Abcam, AB129002, at 1:1000 dilution), rabbit monoclonal GFP antibody (Cell Signaling, 2956S, 1:1000), CD9 antibody (Cell Signaling, 1:2000), monoclonal His antibody (Cell Signaling, 1:1000), and rabbit monoclonal AU1-Tag (Bethyl, 1:2000).

Flow cytometry. Primary PBMCs were transferred into FACS washing buffer and washed twice, and CD8 expression was detected by a human APC-conjugated anti-CD8 antibody (clone RPA-T8, 1:100, BD Biosciences, San Jose, USA). After two additional washing steps, cells were resuspended in 100 μl FACS washing buffer and analyzed by DXP11 flow cytometer. Data were analyzed using the FlowJo 10.0 software (BD Biosciences).

TABLE 1

| Sequence | Sequence | |
|---|---|---|
| Name | Type | Sequence |
| NiV- | DNA | atgaagaagatcaacgagggcctgctggacagcaagatcctgagcgccttcaacaccgtgat |
| Gd34- | | cgccctgctgggcagcatcgtgatcattgtgatgaacatcatgatcatccagaactacaccag |
| G4S-scFv | | aagcaccgacaaccaggccgtgatcaaggacgctctccaggggatccagcagcagatcaag |
| Okt8 | | ggcctggccgacaagatcggcaccgagatcggccccaaggtgtccctgatcgacaccagcag |
| humVh1- | | caccatcaccatccccgccaacatcggcctgctggggtccaagatcagccagagcaccgccag |
| His-tag | | catcaacgagaacgtgaacgagaagtgcaagttcaccctgccccccctgaagatccacgagt |
| (NiV-G- | | gcaacatcagctgccccaaccccctgcccttccgggagtaccggccccagaccgagggcgtga |
| CD8) | | gcaacctggtcggcctgcccaacaacatctgcctgcagaaaaccagcaaccagatcctgaag |
| | | cccaagctcatttcctacaccctgcccgtggtgggccagagcggcacctgcatcaccgaccccc |
| | | tgctggccatggacgagggctacttcgcctacagccacctggaacggatcggcagctgcagca |
| | | ggggcgtgtccaagcagcggatcatcggcgtgggcgaggtgctggaccggggcgacgaggtg |
| | | cccagcctgttcatgaccaacgtgtggaccccccccaaccccaacaccgtgtaccactgcagc |
| | | gccgtgtacaacaacgagttctactacgtgctgtgcgccgtgagcaccgtgggcgaccccatc |
| | | ctgaacagcacctactggtccggcagcctgatgatgacccggctggccgtgaagcctaagagc |
| | | aatggcggcggatacaaccagcaccagctggccctgcggagcatcgagaagggcagatacg |
| | | acaaagtgatgccctacggccccagcggcatcaagcagggcgacacactgtacttccccgcc |
| | | gtgggcttcctggtccggaccgagttcaagtacaacgacagcaactgccccatcaccaagtgc |
| | | cagtacagcaagcccgagaactgcagactgagcatgggcatccggcccaacagccactacat |
| | | cctgcggagcggcctgctgaagtacaacctgagcgacggcgagaaccccaaagtcgtctttat |
| | | tgagatcagcgaccagcggctgtccatcggcagccccagcaagatctacgacagcctgggcc |
| | | agcccgtgttctaccaggccagcttcagctgggacaccatgatcaagttcggcgacgtgctga |
| | | ccgtgaaccccctggtggtgaactggcggaacaataccgtgatcagcagacccggccagagc |
| | | cagtgcccccggttcaacacctgccccgcgatctgcgcggagggcgtgtacaacgacgccttc |
| | | ctgatcgaccggatcaactggatctctgccggcgtgttcctggactccaacgcgaccgccgcga |
| | | atcccgtgttcaccgtgtttaaggacaacgagatcctgtaccgggcccagctggccagcgagg |
| | | acaccaacgcccagaaaaccatcaccaactgctttctgctgaagaacaagatctggtgcatca |
| | | gcctggtggagatctacgataccggcgacaacgtgatcaggcccaagctgttcgccgtgaag |
| | | atccccgagcagtgcaccggtggaggtggctctggtggaggaggctctggaggtggtggatc |
| | | agcggcccagccggcccaggtgcagctggtgcagagcggagccgaggataagaaacctggc |
| | | gctagcgtgaaggtgtcctgcaaggccagcggcttcaacatcaaggacacctacatccactgg |
| | | gtgcgccaggctccaggacagggcctggaatggatgggccggatcgaccccgccaacgaca |
| | | acaccctgtacgccagcaagttccagggcagagtgaccatcaccgccgacaccagcagcaac |
| | | accgcctacatggaactgagcagcctgagaagcgaggacaccgccgtgtactactgtggcag |
| | | aggctacggctactacgtgttcgaccactggggccagggcaccaccgtgacagtgtctagcgg |
| | | cggaggcggaagtggaggcggaggaagcggaggcggcggatccgacatcgtgatgaccca |
| | | gagccctagcagcctgtctgccagcgtgggcgaccgcgtgaccattacctgccggaccagccg |
| | | gtccatcagccagtacctggcctggtatcaggaaaagcccggcaaggcccccaagctgctgat |
| | | ctacagcggcagcaccctgcagagcggcgtgccaagcagattcagcggcagcggctccggca |
| | | ccgacttcaccctgaccatcagcagcctgcagcccgaggacttcgccacctactactgccagca |
| | | gcacaacgagaaccccctgaccttcgggcagggaacaaaggtggagatcaagagagcggcc |
| | | gcaagaggttctcatcaccatcaccatcac (SEQ ID NO: 57) |
| NiV- | PRT | MKKINEGLLDSKILSAFNTVIALLGSIVIIVMNIMIIQNYTRSTDNQAVIKDA |
| Gd34- | | LQGIQQQIKGLADKIGTEIGPKVSLIDTSSTITIPANIGLLGSKISQSTASINEN |
| G4S-scFv | | VNEKCKFTLPPLKIHECNISCPNPLPFREYRPQTEGVSNLVGLPNNICLQKT |
| Okt8 | | SNQILKPKLISYTLPVVGQSGTCITDPLLAMDEGYFAYSHLERIGSCSRGVSK |
| humVh1- | | QRIIGVGEVLDRGDEVPSLFMTNVWTPPNPNTVYHCSAVYNNEFYYVLC |
| His-tag | | AVSTVGDPILNSTYWSGSLMMTRLAVKPKSNGGGYNQHQLALRSIEKGR |
| (NiV-G- | | YDKVMPYGPSGIKQGDTLYFPAVGFLVRTEFKYNDSNCPITKCQYSKPEN |
| CD8) | | CRLSMGIRPNSHYILRSGLLKYNLSDGENPKVVFIEISDQRLSIGSPSKIYDSL |
| | | GQPVFYQASFSWDTMIKFGDVLTVNPLVVNWRNNTVISRPGQSQCPRF |
| | | NTCPAICAEGVYNDAFLIDRINWISAGVFLDSNATAANPVFTVFKDNEILY |
| | | RAQLASEDTNAQKTITNCFLLKNKIWCISLVEIYDTGDNVIRPKLFAVKIPE |
| | | QCTGGGGSGGGGSGGGGSAAQPAQVQLVQSGAEDKKPGASVKVSCKA |
| | | SGFNIKDTYIHWVRQAPGQGLEWMGRIDPANDNTLYASKFQGRVTITA |
| | | DTSSNTAYMELSSLRSEDTAVYYCGRGYGYYVFDHWGQGTTVTVSSGGG |
| | | GSGGGGSGGGGSDIVMTQSPSSLSASVGDRVTITCRTSRSISQYLAWYQE |
| | | KPGKAPKLLIYSGSTLQSGVPSRFSGSGSGTDFTLTISSLQPEDFATYYCQQ |
| | | HNENPLTFGQGTKVEIKRAAARGSHHHHHH (SEQ ID NO: 58) |
| CD8scFv | DNA | Caggtgcagctggtgcagagcggagccgaggataagaaacctggcgctagcgtgaaggtgt |
| | | cctgcaaggccagcggcttcaacatcaaggacacctacatccactgggtgcgccaggctccag |
| | | gacagggcctggaatggatgggccggatcgaccccgccaacgacaacaccctgtacgccagc |
| | | aagttccagggcagagtgaccatcaccgccgacaccagcagcaacaccgcctacatggaact |
| | | gagcagcctgagaagcgaggacaccgccgtgtactactgtggcagaggctacggctactacg |
| | | tgttcgaccactggggccagggcaccaccgtgacagtgtctagcggcggaggcggaagtgga |
| | | ggcggaggaagcggaggcggcggatccgacatcgtgatgacccagagccctagcagcctgtc |
| | | tgccagcgtgggcgaccgcgtgaccattacctgccggaccagccggtccatcagccagtacct |
| | | ggcctggtatcaggaaaagcccggcaaggcccccaagctgctgatctacagcggcagcaccc |
| | | tgcagagcggcgtgccaagcagattcagcggcagcggctccggcaccgacttcaccctgacc |
| | | atcagcagcctgcagcccgaggacttcgccacctactactgccagcagcacaacgagaaccc |
| | | cctgaccttcgggcagggaacaaaggtggagatcaagaga (SEQ ID NO: 59) |
| CD8scFv | PRT | QVQLVQSGAEDKKPGASVKVSCKASGFNIKDTYIHWVRQAPGQGLEW |
| | | MGRIDPANDNTLYASKFQGRVTITADTSSNTAYMELSSLRSEDTAVYYCG |
| | | RGYGYYVFDHWGQGTTVTVSSGGGGSGGGGSGGGGSDIVMTQSPSSLS |
| | | ASVGDRVTITCRTSRSISQYLAWYQEKPGKAPKLLIYSGSTLQSGVPSRFSG |
| | | SGSGTDFTLTISSLQPEDFATYYCQQHNENPLTFGQGTKVEIKR (SEQ ID NO: 60) |
| NiV-G-NT | DNA | atgaagaagatcaacgagggcctgctggacagcaagatcctgagcgccttcaacaccgtgat |
| | | cgccctgctgggcagcatcgtgatcattgtgatgaacatcatgatcatccagaactacaccag |
| | | aagcaccgacaaccaggccgtgatcaaggacgctctccaggggatccagcagcagatcaag |
| | | ggcctggccgacaagatcggcaccgagatcggccccaaggtgtccctgatcgacaccagcag |
| | | caccatcaccatccccgccaacatcggcctgctggggtccaagatcagccagagcaccgccag |
| | | catcaacgagaacgtgaacgagaagtgcaagttcaccctgccccccctgaagatccacgagt |
| | | gcaacatcagctgccccaaccccctgcccttccgggagtaccggccccagaccgagggcgtga |
| | | gcaacctggtcggcctgcccaacaacatctgcctgcagaaaaccagcaaccagatcctgaag |
| | | cccaagctcatttcctacaccctgcccgtggtgggccagagcggcacctgcatcaccgaccccc |
| | | tgctggccatggacgagggctacttcgcctacagccacctggaacggatcggcagctgcagca |
| | | ggggcgtgtccaagcagcggatcatcggcgtgggcgaggtgctggaccggggcgacgaggtg |
| | | cccagcctgttcatgaccaacgtgtggaccccccccaaccccaacaccgtgtaccactgcagc |
| | | gccgtgtacaacaacgagttctactacgtgctgtgcgccgtgagcaccgtgggcgaccccatc |
| | | ctgaacagcacctactggtccggcagcctgatgatgacccggctggccgtgaagcctaagagc |
| | | aatggcggcggatacaaccagcaccagctggccctgcggagcatcgagaagggcagatacg |
| | | acaaagtgatgccctacggccccagcggcatcaagcagggcgacacactgtacttccccgcc |
| | | gtgggcttcctggtccggaccgagttcaagtacaacgacagcaactgccccatcaccaagtgc |
| | | cagtacagcaagcccgagaactgcagactgagcatgggcatccggcccaacagccactacat |
| | | cctgcggagcggcctgctgaagtacaacctgagcgacggcgagaaccccaaagtcgtctttat |
| | | tgagatcagcgaccagcggctgtccatcggcagccccagcaagatctacgacagcctgggcc |
| | | agcccgtgttctaccaggccagcttcagctgggacaccatgatcaagttcggcgacgtgctga |
| | | ccgtgaaccccctggtggtgaactggcggaacaataccgtgatcagcagacccggccagagc |
| | | cagtgcccccggttcaacacctgccccgcgatctgcgcggagggcgtgtacaacgacgccttc |
| | | ctgatcgaccggatcaactggatctctgccggcgtgttcctggactccaacgcgaccgccgcga |
| | | atcccgtgttcaccgtgtttaaggacaacgagatcctgtaccgggcccagctggccagcgagg |
| | | acaccaacgcccagaaaaccatcaccaactgctttctgctgaagaacaagatctggtgcatca |
| | | gcctggtggagatctacgataccggcgacaacgtgatcaggcccaagctgttcgccgtgaag |
| | | atccccgagcagtgcaccggtggaggtggctctggtggaggaggctctggaggtggtggatc |
| | | agcggcccagccggccgcggccgcaagaggttctcatcaccatcaccatcac (SEQ ID |
| | | NO: 61) |
| NiV-G-NT | PRT | MKKINEGLLDSKILSAFNTVIALLGSIVIIVMNIMIIQNYTRSTDNQAVIKDA |
| | | LQGIQQQIKGLADKIGTEIGPKVSLIDTSSTITIPANIGLLGSKISQSTASINEN |
| | | VNEKCKFTLPPLKIHECNISCPNPLPFREYRPQTEGVSNLVGLPNNICLQKT |
| | | SNQILKPKLISYTLPVVGQSGTCITDPLLAMDEGYFAYSHLERIGSCSRGVSK |
| | | QRIIGVGEVLDRGDEVPSLFMTNVWTPPNPNTVYHCSAVYNNEFYYVLC |
| | | AVSTVGDPILNSTYWSGSLMMTRLAVKPKSNGGGYNQHQLALRSIEKGR |
| | | YDKVMPYGPSGIKQGDTLYFPAVGFLVRTEFKYNDSNCPITKCQYSKPEN |
| | | CRLSMGIRPNSHYILRSGLLKYNLSDGENPKVVFIEISDQRLSIGSPSKIYDSL |
| | | GQPVFYQASFSWDTMIKFGDVLTVNPLVVNWRNNTVISRPGQSQCPRF |
| | | NTCPAICAEGVYNDAFLIDRINWISAGVFLDSNATAANPVFTVFKDNEILY |
| | | RAQLASEDTNAQKTITNCFLLKNKIWCISLVEIYDTGDNVIRPKLFAVKIPE |
| | | QCTGGGGSGGGGSGGGGSAAQPAAAARGSHHHHHH (SEQ ID NO: 62) |

### REFERENCES

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

### EQUIVALENTS AND SCOPE

Those skilled in the art will recognize or be able to ascertain using no more than routine experimentation many equivalents to the specific embodiments of the invention described herein. The scope of the present invention is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity, those embodiments have not been specifically set forth in haec verba herein. Thus, for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. An arrestin domain-containing protein 1 (ARRDC1)-mediated microvesicle (ARMM), comprising:
(i) a lipid bilayer and an ARRDC1 protein or a variant thereof,
(ii) a Nipah virus (NiV) glycoprotein variant fused to an antibody or antigen-binding fragment, wherein the NiV glycoprotein variant does not recognize and/or bind to an ephrin receptor, and
(iii) a NiV fusion protein.

2. The microvesicle of claim 1, wherein the ARRDC1 protein comprises an amino acid sequence at least 80%, at least 85%, at least 90%, at least 95%, at least 96%, at least 97%, at least 98%, at least 99%, or 100% identical to the amino acid sequence of SEQ ID NO: 4.

3. The microvesicle of claim 1 or 2, wherein the NiV glycoprotein variant fused to an antibody or antigen binding fragment comprises an amino acid sequence at least 80%, at least 85%, at least 90%, at least 95%, at least 96%, at least 97%, at least 98%, at least 99%, or 100% identical to the amino acid sequence of SEQ ID NO: 58.

4. The microvesicle of any one of claims 1-3, wherein the NiV fusion protein comprises an amino acid sequence at least 80%, at least 85%, at least 90%, at least 95%, at least 96%, at least 97%, at least 98%, at least 99%, or 100% identical to the amino acid sequence SEQ ID NOs: 69.

5. The microvesicle of any one of claims 1-4, wherein the antibody or antigen binding fragment comprises a CD8 targeting antibody or antigen binding fragment.

6. The microvesicle of any one of claims 1-5, wherein the antigen-binding fragment comprises an amino acid sequence at least 80%, at least 85%, at least 90%, at least 95%, at least 96%, at least 97%, at least 98%, at least 99%, or 100% identical to the amino acid sequence of SEQ ID NO: 60.

7. The microvesicle of any one of claims 1-6, wherein the antibody or antigen-binding fragment comprises a single-chain variable fragment (scFv).

8. The microvesicle of claim 1-6, wherein the antibody or antigen-binding fragment comprises a nanobody.

9. The microvesicle of claim 1-6, wherein the antibody or antigen-binding fragment comprises a designed ankyrin repeat protein (DARPin).

10. The microvesicle of any one of claims 1-9, wherein the antibody or antigen-binding fragment targets a particular cell type.

11. The microvesicle of claim 10, wherein the cell type is selected from the group consisting of neurons, astrocytes, oligodendrocytes, microglial cells, T-cells, dendritic cells, B cells, NK cells, stem cells, progenitor cells, endothelial cells, muscle cells, myocardial cells, epithelial cells, and hepatic cells.

12. The microvesicle of any one of claims 1-11, wherein the antibody or antigen-binding fragment targets T-cells.

13. The microvesicle of any one of claim 1-4 or 7-12, wherein the antibody or antigen-binding fragment targets neurons.

14. The microvesicle of any one of claims 1-13, wherein the antibody or antigen-binding fragment binds selectively to an antigen expressed by target cell.

15. The microvesicle of claim 14, wherein the target cell is a CD8 expressing cell.

16. The microvesicle of claim 14 or 15, wherein the target cell is a T cell.

17. The microvesicle of any one of claims 1-16, wherein the NiV glycoprotein variant comprises one or more amino acid substitutions relative to a wild-type NiV glycoprotein of SEQ ID NO: 1.

18. The microvesicle of claim 17, wherein at least one of the one or more amino acid substitutions are selected from the group consisting of Y389X, E501X, W504X, Q530X, E533X, and I588X, wherein X is any amino acid.

19. The microvesicle of claim 17 or 18, wherein at least one of the one or more amino acid substitutions are selected from the group consisting of Y389A, E501A, W504A, Q530A, E533A, and I588A.

20. The microvesicle of any one of claims 17-19, wherein the NiV glycoprotein variant comprises amino acid substitutions E501A, W504A, Q530A, and E533A relative to a wild-type NiV glycoprotein of SEQ ID NO: 1.

21. The microvesicle of any one of claims 17-20, wherein the NiV glycoprotein variant further comprises a C-terminal truncation.

22. The microvesicle of any one of claims 17-21, wherein the NiV glycoprotein variant further comprises a C-terminal truncation of about 33 or about 34 amino acids in length.

23. The microvesicle of any one of claims 1-22, wherein the NiV fusion protein comprises a C-terminal truncation.

24. The microvesicle of any one of claims 1-23, wherein the NiV fusion protein comprises a C-terminal truncation of about 22 amino acids in length.

25. The microvesicle of any one of claims 1-24, wherein the NiV glycoprotein variant comprises an amino acid sequence at least 80%, at least 85%, at least 90%, at least 95%, at least 96%, at least 97%, at least 98%, or at least 99% identical to the amino acid sequence of any one of SEQ ID NOs: 63-68.

26. The microvesicle of any one of claims 1-25, wherein the NiV glycoprotein variant comprises the amino acid sequence of any one of SEQ ID NOs: 63-68.

27. The microvesicle of any one of claims 1-26, wherein the NiV fusion protein comprises an amino acid sequence at least 80%, at least 85%, at least 90%, at least 95%, at least 96%, at least 97%, at least 98%, or at least 99% identical to the amino acid sequence of SEQ ID NO: 6.

28. The microvesicle of any one of claims 1-27, wherein the NiV fusion protein comprises the amino acid sequence of SEQ ID NO: 6.

29. The microvesicle of any one of claims 1-28, wherein the ARRDC1 protein comprises an amino acid sequence at least 80%, at least 85%, at least 90%, at least 95%, at least 96%, at least 97%, at least 98%, or at least 99% identical to the amino acid sequence of any one of SEQ ID NOs: 3-5.

30. The microvesicle of any one of claims 1-29, wherein the ARRDC1 protein comprises the amino acid sequence of any one of SEQ ID NOs: 3-5.

31. The microvesicle of any one of claims 1-30, further comprising an agent.

32. The microvesicle of claim 31, wherein the agent is fused to the ARRDC1 protein.

33. The microvesicle of claim 31, wherein the ARRDC1 protein is fused to TAR.

34. The microvesicle of claim 33, wherein TAR recruits an agent fused to Tat, optionally wherein the agent is an RNA.

35. The microvesicle of claim 31, wherein the agent is fused to a WW domain, optionally wherein the agent is a protein.

36. The microvesicle of claim 35, wherein the WW domain comprises an amino acid sequence of any one of SEQ ID NOs: 7-44, or an amino acid sequence at least 80%, at least 85%, at least 90%, at least 95%, at least 96%, at least 97%, at least 98%, or at least 99% identical to the amino acid sequence of any one of SEQ ID NOs: 7-44.

37. The microvesicle of claim 31, wherein the agent is selected from the group consisting of a nucleic acid, a protein, and a small molecule.

38. The microvesicle of claim 37, wherein the nucleic acid comprises an RNA.

39. The microvesicle of claim 37 or 38, wherein the nucleic acid comprises an RNAi agent.

40. The microvesicle of any one of claims 37-39, wherein the nucleic acid comprises a coding RNA, a non-coding RNA, an antisense RNA, an mRNA, a small RNA, an siRNA, an shRNA, a microRNA, an snRNA, a snoRNA, a lincRNA, a structural RNA, a ribozyme, or a precursor of any thereof.

41. The microvesicle of claim 37, wherein the nucleic acid comprises a DNA.

42. The microvesicle of claim 41, wherein the DNA comprises a retrotransposon sequence, a LINE sequence, a SINE sequence, a composite SINE sequence, or an LTR-retrotransposon sequence.

43. The microvesicle of any one of claims 37-42, wherein the nucleic acid encodes a protein.

44. The microvesicle of claim 37, wherein the agent comprises a protein.

45. The microvesicle of claim 44, wherein the protein is selected from the group consisting of an enzyme, an antibody, a Fab, a Fab′, a F(ab′)2, a Fd, a scFv, a Fv, a dsFv, a diabody, and an affibody.

46. The microvesicle of claim 44, wherein the protein comprises a transcription factor, a transcriptional repressor, a fluorescent protein, a kinase, a phosphatase, a protease, a ligase, or a recombinase.

47. The microvesicle of any one of claims 44-46, wherein the protein is covalently bound to the ARRDC1 protein.

48. The microvesicle of any one of claims 44-47, wherein the protein is conjugated to the ARRDC1 protein via a linker.

49. The microvesicle of claim 48, wherein the linker is a cleavable linker.

50. The microvesicle of claim 49, wherein the linker comprises a protease recognition site or a UV-cleavable moiety.

51. The microvesicle of claim 44, wherein the protein is a fusion protein.

52. The microvesicle of claim 44, wherein the protein is a nuclease.

53. The microvesicle of claim 44, wherein the protein is a zinc finger nuclease (ZFN).

54. The microvesicle of claim 44, wherein the protein is a TALEN.

55. The microvesicle of claim 44, wherein the protein is a Cas protein, or a variant thereof.

56. The microvesicle of claim 44, wherein the protein is a Cas9 protein, or a variant thereof.

57. The microvesicle of claim 56, wherein the Cas9 protein is a Cas9 nickase (nCas9).

58. The microvesicle of claim 56, wherein the Cas9 protein is a nuclease-inactived Cas9 protein (dCas9).

59. The microvesicle of any one of claims 56-58, wherein the Cas9 protein or variant thereof is fused to at least one nuclear localization sequence (NLS).

60. The microvesicle of claim 51, wherein the fusion protein comprises a nuclease.

61. The microvesicle of claim 51, wherein the fusion protein comprises a ZFN.

62. The microvesicle of claim 51, wherein the fusion protein comprises a TALEN.

63. The microvesicle of claim 51, wherein the fusion protein comprises a Cas protein.

64. The microvesicle of any one of claims 51-70, further comprising a guide RNA (gRNA).

65. The microvesicle of any one of claims 31-64, wherein the agent comprises a detectable label.

66. The microvesicle of any one of claims 31-65, wherein the agent comprises a therapeutic agent.

67. The microvesicle of any one of claims 31-65, wherein the agent comprises a cytotoxic agent.

68. One or more isolated nucleic acids encoding the ARRDC1 protein, the NiV glycoprotein variant, and the NiV fusion protein of the microvesicle of any one of claims 1-67.

69. One or more vectors comprising the one or more isolated nucleic acids of claim 68.

70. A vector comprising the isolated nucleic acid of claim 68.

71. A microvesicle-producing cell comprising:
a first isolated nucleic acid encoding an ARRDC1 protein or a variant thereof,
a second isolated nucleic acid encoding a Nipah virus (NiV) glycoprotein variant fused to an antibody or antigen-binding fragment, wherein the NiV glycoprotein variant does not recognize and/or bind to an ephrin receptor, and
a third isolated nucleic acid encoding a NiV fusion protein.

72. The microvesicle of claim 71, wherein the ARRDC1 protein comprises an amino acid sequence at least 80%, at least 85%, at least 90%, at least 95%, at least 96%, at least 97%, at least 98%, at least 99%, or 100% identical to the amino acid sequence of SEQ ID NO: 4.

73. The microvesicle of claim 71 or 72, wherein the NiV glycoprotein variant fused to an antibody or antigen binding fragment comprises an amino acid sequence at least 80%, at least 85%, at least 90%, at least 95%, at least 96%, at least 97%, at least 98%, at least 99%, or 100% identical to the amino acid sequence of SEQ ID NO: 58.

74. The microvesicle of any one of claims 71-73, wherein the NiV fusion protein comprises an amino acid sequence at least 80%, at least 85%, at least 90%, at least 95%, at least 96%, at least 97%, at least 98%, at least 99%, or 100% identical to the amino acid sequence of SEQ ID NO: 69.

75. The microvesicle of any one of claims 71-74, wherein the antibody or antigen binding fragment comprises a CD8 targeting antibody or antigen binding fragment.

76. The microvesicle of any one of claims 71-76, wherein the antigen-binding fragment comprises an amino acid sequence at least 80%, at least 85%, at least 90%, at least 95%, at least 96%, at least 97%, at least 98%, at least 99%, or 100% identical to the amino acid sequence of SEQ ID NO: 60.

77. The microvesicle-producing cell of any one of claims 71-76, wherein the first isolated nucleic acid, the second isolated nucleic acid, and the third isolated nucleic acid are expressed on the same recombinant expression construct under the control of one or more heterologous promoters.

78. The microvesicle-producing cell of any one of claims 71-76, wherein the first isolated nucleic acid, the second isolated nucleic acid, and the third isolated nucleic acid are expressed on more than one recombinant expression construct, wherein the more than one recombinant expression constructs are under the control of one or more heterologous promoters.

79. The microvesicle-producing cell of any one of claims 71-76, wherein the first isolated nucleic acid, the second isolated nucleic acid, and the third isolated nucleic acid are each expressed on different recombinant expression constructs, wherein the different recombinant expression constructs are each under the control of a heterologous promoter.

80. The microvesicle-producing cell any one of claims 71-79, wherein the antibody or antigen-binding fragment comprises a single-chain variable fragment (scFv).

81. The microvesicle-producing cell of any one of claims 71-79, wherein the antibody or antigen-binding fragment comprises a nanobody.

82. The microvesicle-producing cell of any one of claims 71-79, wherein the antibody or antigen-binding fragment comprises a designed ankyrin repeat protein (DARPin).

83. The microvesicle-producing cell of any one of claims 71-82, wherein the antibody or antigen-binding fragment targets a particular cell type.

84. The microvesicle-producing cell of claim 83, wherein the cell type is selected from the group consisting of neurons, astrocytes, oligodendrocytes, microglial cells, T cells, dendritic cells, B cells, NK cells, stem cells, progenitor cells, endothelial cells, muscle cells, myocardial cells, epithelial cells, and hepatic cells.

85. The microvesicle-producing cell any one of claims 71-84, wherein the antibody or antigen-binding fragment targets T-cells.

86. The microvesicle-producing cell of any one of claims 71-84, wherein the antibody or antigen-binding fragment targets neurons.

87. The microvesicle-producing cell of any one of claims 71-86, wherein the antibody or antigen-binding fragment binds selectively to an antigen expressed by target cell.

88. The microvesicle-producing cell of claim 87, wherein the antibody or antigen-binding fragment selectively binds to CD8.

89. The microvesicle-producing cell of claim 88, wherein the antibody or antigen-binding fragment is an anti-CD8 antibody or antigen-binding fragment.

90. The microvesicle-producing cell of any one of claims 71-89, wherein the NiV glycoprotein variant comprises one or more amino acid substitutions relative to a wild-type NiV glycoprotein of SEQ ID NO: 1.

91. The microvesicle-producing cell of claim 90, wherein at least one of the one or more amino acid substitutions are selected from the group consisting of Y389X, E501X, W504X, Q530X, E533X, and I588X, wherein X is any amino acid.

92. The microvesicle-producing cell of claim 90 or 91, wherein at least one of the one or more amino acid substitutions are selected from the group consisting of Y389A, E501A, W504A, Q530A, E533A, and I588A.

93. The microvesicle-producing cell of any one of claims 90-92, wherein at least one of the NiV glycoprotein variant comprises amino acid substitutions E501A, W504A, Q530A, and E533A relative to a wild-type NiV glycoprotein of SEQ ID NO: 1.

94. The microvesicle-producing cell of any one of claims 71-93, wherein the NiV glycoprotein variant further comprises a C-terminal truncation.

95. The microvesicle-producing cell of any one of claims 71-94, wherein the NiV glycoprotein variant further comprises a C-terminal truncation of about 33 or about 34 amino acids in length.

96. The microvesicle-producing cell of any one of claims 71-95, wherein the NiV fusion protein comprises a C-terminal truncation.

97. The microvesicle-producing cell of any one of claims 71-96, wherein the NiV fusion protein comprises a C-terminal truncation of about 22 amino acids in length.

98. The microvesicle-producing cell of any one of claims 71-97, wherein the NiV glycoprotein variant comprises an amino acid sequence at least 80%, at least 85%, at least 90%, at least 95%, at least 96%, at least 97%, at least 98%, or at least 99% identical to the amino acid sequence of any one of SEQ ID NOs: 63-68.

99. The microvesicle-producing cell of any one of claims 71-98, wherein the NiV glycoprotein variant comprises the amino acid sequence of any one of SEQ ID NOs: 63-68.

100. The microvesicle-producing cell of any one of claims 71-99, wherein the NiV fusion protein comprises an amino acid sequence at least 80%, at least 85%, at least 90%, at least 95%, at least 96%, at least 97%, at least 98%, or at least 99% identical to the amino acid sequence of SEQ ID NO: 69.

101. The microvesicle-producing cell of any one of claims 71-100, wherein the NiV fusion protein comprises the amino acid sequence of SEQ ID NO: 69.

102. The microvesicle-producing cell of any one of claims 71-101, wherein the ARRDC1 protein comprises an amino acid sequence at least 80%, at least 85%, at least 90%, at least 95%, at least 96%, at least 97%, at least 98%, or at least 99% identical to the amino acid sequence of any one of SEQ ID NOs: 3-5.

103. The microvesicle-producing cell of any one of claims 71-102, wherein the ARRDC1 protein comprises the amino acid sequence of any one of SEQ ID NOs: 3-5.

104. A method of delivering a molecule to a target cell, the method comprising contacting the target cell with the microvesicle of any of claims 1-67.

105. The method of claim 104, wherein the target cells are T-cells.

106. A method of treating a patient, the method consisting of administering to the patient a microvesicle of any one of claims 1-67.

107. A method of treating a patient, the method consisting of administering to the patient a microvesicle-producing cell of any one of claims 71-103.

108. A method of delivering an arrestin domain-containing protein 1 (ARRDC1)-mediated microvesicle (ARMM), comprising: delivering the ARMM of any one of claims 1-67, or the microvesicle-producing cell of any one of claims 71-94, to a subject.

109. The method of claim 108, wherein the subject is mammalian.

110. The method of claim 108 or 109, wherein the subject is human.

111. A kit comprising one or more of the ARMMs of any one of claims 1-67, or the microvesicle-producing cells of any one of claims 71-94.
